SEQUENCE LISTING (43 sequences)

SEQ ID NO: 1 - DNA, 63 bp, Artificial Sequence
  Description of Artificial Sequence Synthetic oligonucleotide
tggaggatcgccatgcaccaccaccaccaccaccacgctagcgcgggcagatcttgaggccgg

SEQ ID NO: 2 - DNA, 63 bp, Artificial Sequence
  Description of Artificial Sequence Synthetic oligonucleotide
cctcaagatctgcccgcgctagcgtggtggtggtggtggtggtgcatggcgatcctccacatg

SEQ ID NO: 3 - DNA, 84 bp, Artificial Sequence
  Description of Artificial Sequence Synthetic oligonucleotide
tggaggatcgccatgcaccaccaccaccaccaccacgagaacctgtacttccagtccgctagctagggaagatcttgaggccgg

SEQ ID NO: 4 - DNA, 84 bp, Artificial Sequence
  Description of Artificial Sequence Synthetic oligonucleotide
cctcaagatcttccctagctagcggactggaagtacaggttctcgtggtggtggtggtggtggtgcatggcgatcctccacatg

SEQ ID NO: 5 - DNA, 62 bp, Artificial Sequence
  Description of Artificial Sequence Synthetic oligonucleotide
ctagttccatatgatagatctcaccaccaccaccaccaccaccaccaccactaataggccgg

SEQ ID NO: 6 - DNA, 54 bp, Artificial Sequence
  Description of Artificial Sequence Synthetic oligonucleotide
aaggtatactatctagagtggtggtggtggtggtggtggtggtggtgattatcc

SEQ ID NO: 7 - DNA, 45 bp, Artificial Sequence
  Description of Artificial Sequence Synthetic oligonucleotide
aacccacgccaccagtaggcaggaggaacacgtgtcgtccggtgg

SEQ ID NO: 8 - DNA, 32 bp, Artificial Sequence
  Description of Artificial Sequence Synthetic oligonucleotide
gaccaccggacgacacgtgttcctcctgccta

SEQ ID NO: 9 - DNA, 32 bp, Artificial Sequence
  Description of Artificial Sequence Synthetic oligonucleotide
gcctattccaatcctacgtagaagggaagatc

SEQ ID NO: 10 - DNA, 33 bp, Artificial Sequence
  Description of Artificial Sequence Synthetic oligonucleotide
aagatcttcccttctacgtaggattggaatagg

SEQ ID NO: 11 - DNA, 44 bp, Artificial Sequence
  Description of Artificial Sequence Synthetic oligonucleotide
gagaacctgtacttccaatcctttacgtagaaatagggaagatc

SEQ ID NO: 12 - DNA, 45 bp, Artificial Sequence
  Description of Artificial Sequence Synthetic oligonucleotide
aagatcttccctatttctacgtaaaggattggaagtacaggttct

SEQ ID NO: 13 - DNA, 14386 bp, Artificial Sequence
  Description of Artificial Sequence Synthetic plasmid
ccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcggccgcgggctggccgaggtgctgggcaagccctacctccaggcccccatcggggtcgagagcacgaccgccttcctgcgccgcctgggcgagattctgggcctcgatccggagcccttcatcgagcgcgagaagcactcgacgctgaagcccgtgtgggatctgtggcggagtgtcacgcaggacttcttcgggacggccaatttcggaatcgtggcgaccgaaacttatgcaagaggcatccgaaactatctcgaaggcgatctcgggctgccctgcgccttcgccgtggcccgcaagaggggctcgaagaccgacaacgaagcggtgcgcggactgatccgccagcaccgtccgctcgtgctcatggggtcgatcaacgagaagatttaccttgcggaactgaaagccggtcacggcccgcaaccctctttcatcgctgcctctttcccgggtgcggcgatccggcgcgctaccggaacgcccgttatgggatatgcaggtgctacgtggttactgcaggaagtttgcaacgccctgttcgacgccctgttccacattctgcccctcgggacggagatggacagcgccgccgccacaccgacgacactgcgccgcgacttcccgtgggatgccgatgcgcaagcggccctggaccgcatcgtagaggagcatccggttctcacccggatcagcgccgcgcgtgccttgcgcgacgccgccgagaaggctgccctcgatgccggtgccgagagggtcgtgagagagactgtcgaagccctgcgtgggccgggcttcggcgagaggaagggagagaaccaatgagcgatcatgccgtcaacacgccggtccatgccgccagggcccacgggcaccgagcaccacgtgccgagttctacgtctacttcgccgtcattctgctgggcgccttcccggtggccttcgtgagctggatcgtctcgacgatccgccaccgcaggcttcccaagcgcggccccttcgcgtccgcctggttcgatgccaaggcgatcacgccgctgattttccgcgcctgaccgcaggtcaggttgcgacacgccattcgtcgtctccccaaggggcggcggattaatcgggagggcatggtgccttaccgtaacccacgccaccagcatgtggaggatcgccatgcaccaccaccaccaccaccacgcctattccaatcctacgtagaagggaagatcttgagggccggccctccgtcgcgggcggcacccacgcccgcatcgattccaaggttcagccattgagacggctccgcttcgcgcgcaagcgcgggttgggccgactgcaagcggagagggaagcatggcactgctcagcttcgagcgaaaatatcgcgtgccggggggcacgctggtcggcggaaacctgttcgacttctgggtcggccctttctatgtcggcttcttcggggttgcgacgtttttcttcgcggccctgggtatcattctgattgcctggagtgccgtactccagggtacctggaacccccaactcatctctgtctacccgccggcccttgaatatggcctgggaggtgcacccctcgcaaaaggcgggctgtggcagatcatcacgatctgcgccactggtgccttcgtcagctgggcgctgcgcgaagtcgaaatctgccgtaagctgggcatcgggtaccacatcccgttcgccttcgcgttcgccatcctggcctacctgacgctggtgctgttccgcccggtgatgatgggcgcctggggctatgccttcccctacgggatctggacgcacctcgactgggtgtcgaacacgggctacacctacggcaacttccactacaaccctgcccacatgatcgccatctcgttcttcttcacgaacgcgctggctctggcgctgcacggcgcccttgtgctctccgcggccaaccccgagaagggcaaggaaatgcggacgccggatcacgaggatacgttcttccgcgatctggtcggctactcgatcgggacgctcggcatccaccgcctcggcctgctgctctcgctgagcgccgtcttcttcagcgccctctgcatgatcattaccggcaccatctggttcgatcagtgggtcgactggtggcaatggtgggtgaagctgccgtggtgggcgaacatcccgggaggcatcaatggctgagtatcagaacatcttctcccaggtccaggtccgcggaccggccgacctggggatgaccgaagacgtcaacctggccaaccgttcgggcgtcggtcccttctcgaccctgctcggctggttcggcaacgcccagctcggcccgatctatctcggctcgctcggcgtcctgtccctcttctcgggcctgatgtggttcttcaccatcgggatctggttctggtatcaggcgggctggaacccggccgtcttcctgcgcgacctgttcttcttctcgctcgagccgccggcacccgaatacggtctgtccttcgcggctccgctgaaggaaggcgggctgtggctgatcgcgtcgttcttcatgttcgtcgcggtctggtcctggtggggccgcacctatctccgcgctcaggcgctgggcatgggcaagcacaccgcctgggcgttcctctcggccatctggctgtggatggtgctgggcttcatccgtccgatcctcatggggtcctggtcggaagcggttccctacggcatcttctcgcacctcgactggacgaacaacttctcgctcgtccacggcaacctgttctacaaccccttccacggtctctcgatcgccttcctctacgggtcggccctgctcttcgcgatgcacggtgcgaccatcctcgcggtctcccgcttcggcggcgagcgcgagctggagcagatcgccgaccgcgggacggcagcggagcgggccgccctcttctggcgctggaccatgggtttcaacgccacgatggaaggcatccaccgctgggccatctggatggcggtcctcgtgaccctcaccggcggcatcgggatcctgctctcgggcacggtcgtggacaactggtacgtctggggccagaaccacggcatggcgccgctgaactgaggagcgatcacaatggctgacaagaccatcttcaacgatcacctcaacaccaatccgaagaccaaccttcgcctctgggtcgctttccagatgatgaagggtgcgggctgggctggcggcgtgttcttcgggacgctccttctcatcgggttcttccgggtggtcgggcggatgcttccgatccaggagaaccaggctccggcgccgaacatcaccggcgctctggagaccgggatcgagctgatcaagcatctcgtctgagacaagtctcggggcagggcggcgcgaggccgcccgctcctccaagtccgggccatatcgccggcccgggtccggggcgacaccacagcccggttcccttcctgttggcgacagggacctggtgccgtgtggaagaccgcacggcacccttttgacattcacgggaggctctgatgaccaatcccaccccgcgacccgaaaccccgcttttggatcgcgtctgctgcccggccgacatgaaggcgctgagtgacgccgaactggagcggctggccgacgaagtgcgttccgaggtcagtgataggggtagtttcttattttaggcagtttatatgaaattaagacatgcagatgtcacagtggatattgaactggtctcgaaagctcaatatcccccaaagcacaagcacaaacttcgacatcatgcagaagcgtttcccgaaccgcgtcttcgacgtgggcatcgccgagcagcatgccgtgaccttcgcggccggcctcgccggggccgggatgaagcccttctgcgcgatctattcctcgttcctgcaacggggttacgaccagatcgcccatgacgtggcgctgcagaaccttcccgtccgcttcgtgatcgaccgggcggggctcgtgggggccgatggcgcgacccatgcgggggccttcgacgttggcttcatcacttcgctgcccaacatgaccgtgatggccgcggccgacgaggccgagctcatccacatgatcgccaccgccgtggccttcgacgagggccccatcgccttccgcttcccgcggggcgagggggtgggcgtcgagatgcccgagcgcgggacggtgctggagcccggccggggccgcgtggtgcgcgaagggacggatgtcgcgatcctctccttcggcgcgcatctgcacgaggccttgcaggcggcgaaacttctcgaggccgagggggtgagcgtgaccgtggccgacgcccgcttctcgcgcccgctcgacacggggctcatcgaccagctcgtgcgccatcacgcggcgctggtaacggtggagcagggggccatgggcggcttcggcgcccatgtcatgcactatctcgccaattccggcggcttcgacgggggcctcgcgctccgggtcatgacgctgcccgaccgcttcatcgagcaggcgagccccgaggacatgtatgccgatgcggggctgcgggccgaggatatcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgcgttcaatcggaccagcggaggctggtccggaggccagacatgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccactgtcgactacgccatcatggcgacagcgcctttcctttgggttctctatatcgggcggatcgtggccggcatcaccggggcgactggggcggtagccggcgcttatattgccgatatcactgatggcgatgagcgcgcgcggcacttcggcttcatgagcgcctgtttcgggttcgggatggtcgcgggacctgtgctcggtgggctgatgggcggtttctccccccacgctccgttcttcgccgcggcagccttgaacggcctcaatttcctgacgggctgtttccttttgccggagtcgcacaaaggcgaacgccggccgttacgccgggaggctctcaacccgctcgcttcgttccggtgggcccggggcatgaccgtcgtcgccgccctgatggcggtcttcttcatcatgcaacttgtcggacaggtgccggccgcgctttgggtcattttcggcgaggatcgctttcactgggacgcgaccacgatcggcatttcgcttgccgcatttggcattctgcattcactcgcccaggcaatgatcaccggccctgtagccgcccggctcggcgaaaggcgggcactcatgctcggaatgattgccgacggcacaggctacatcctgcttgccttcgcgacacggggatggatggcgttcccgatcatggtcctgcttgcttcgggtggcatcggaatgccggcgctgcaagcaatgttgtccaggcaggtggatgaggaacgtcaggggcagctgcaaggctcactggcggcgctcaccagcctgacctcgatcgtcggacccctcctcttcacggcgatctatgcggcttctataacaacgtggaacgggtgggcatggattgcaggcgctgccctctacttgctctgcctgccggcgctgcgtcgcgggctttggagcggcgcagggcaacgagccgatcgctgatcgtggaaacgataggcctatgccatgcgggtcaaggcgacttccggcaagctatacgcgccctaggagtgcggttggaacgttggcccagccagatactcccgatcacgagcaggacgccgatgatttgaagcgcactcagcgtctgatccaagaacaaccatcctagcaacacggcggtccccgggctgagaaagcccagtaaggaaacaactgtaggttcgagtcgcgagatcccccggaaccaaaggaagtaggttaaacccgctccgatcaggccgagccacgccaggccgagaacattggttcctgtaggcatcgggattggcggatcaaacactaaagctactggaacgagcagaagtcctccggccgccagttgccaggcggtaaaggtgagcagaggcacgggaggttgccacttgcgggtcagcacggttccgaacgccatggaaaccgcccccgccaggcccgctgcgacgccgacaggatctagcgctgcgtttggtgtcaacaccaacagcgccacgcccgcagttccgcaaatagcccccaggaccgccatcaatcgtatcgggctacctagcagagcggcagagatgaacacgaccatcagcggctgcacagcgcctaccgtcgccgcgaccccgcccggcaggcggtagaccgaaataaacaacaagctccagaatagcgaaatattaagtgcgccgaggatgaagatgcgcatccaccagattcccgttggaatctgtcggacgatcatcacgagcaataaacccgccggcaacgcccgcagcagcataccggcgacccctcggcctcgctgttcgggctccacgaaaacgccggacagatgcgccttgtgagcgtccttggggccgtcctcctgtttgaagaccgacagcccaatgatctcgccgtcgatgtaggcgccgaatgccacggcatctcgcaaccgttcagcgaacgcctccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggtcgtggccgcgccaacctttgcgatccgcaagcgcgcggtcgccatcttcacgctggaacagtacgtcgaggcgggcatcatgacccgcgagcaatacgaggtcattaaaagcgccgtgattgatgatatagcggcccggctgctcctggttctcgcgcaccgaaatgggtgacttcaccccgcgctctttgatcgtggcaccgatttccgcgatgctctccggggaaaagccggggttgtcggccgtccgcggctgatgcggatcttcgtcgatcaggtccaggtccagctcgatagggccggaaccgccctgagacgccgcaggagcgtccaggaggctcgacaggtcgccgatgctatccaaccccaggccggacggctgcgccgcgcctgcggcttcctgagcggccgcagcggtgtttttcttggtggtcttggcttgagccgcagtcattgggaaatctccatcttcgtgaacacgtaatcagccagggcgcgaacctctttcgatgccttgcgcgcggccgttttcttgatcttccagaccggcacaccggatgcgagggcatcggcgatgctgctgcgcaggccaacggtggccggaatcatcatcttggggtacgcggccagcagctcggcttggtggcgcgcgtggcgcggattccgcgcatcgaccttgctgggcaccatgccaaggaattgcagcttggcgttcttctggcgcacgttcgcaatggtcgtgaccatcttcttgatgccctggatgctgtacgcctcaagctcgatgggggacagcacatagtcggccgcgaagagggcggccgccaggccgacgccaagggtcggggccgtgtcgatcaggcacacgtcgaagccttggttcgccagggccttgatgttcgccccgaacagctcgcgggcgtcgtccagcgacagccgttcggcgttcgccagtaccgggttggactcgatgagggcgaggcgcgcggcctggccgtcgccggctgcgggtgcggtttcggtccagccgccggcagggacagcgccgaacagcttgcttgcatgcaggccggtagcaaagtccttgagcgtgtaggacgcattgccctgggggtccaggtcgatcacggcaacccgcaagccgcgctcgaaaaagtcgaaggcaagatgcacaagggtcgaagtcttgccgacgccgcctttctggttggccgtgaccaaagttttcatcgtttggtttcctgttttttcttggcgtccgcttcccacttccggacgatgtacgcctgatgttccggcagaaccgccgttacccgcgcgtacccctcgggcaagttcttgtcctcgaacgcggcccacacgcgatgcaccgcttgcgacactgcgcccctggtcagtcccagcgacgttgcgaacgtcgcctgtggcttcccatcgactaagacgccccgcgctatctcgatggtctgctgccccacttccagcccctggatcgcctcctggaactggctttcggtaagccgtttcttcatggataacacccataatttgctccgcgccttggttgaacatagcggtgacagccgccagcacatgagagaagtttagctaaacatttctcgcacgtcaacacctttagccgctaaaactcgtccttggcgtaacaaaacaaaagcccggaaaccgggctttcgtctcttgccgcttatggctctgcacccggctccatcaccaacaggtcgcgcacgcgcttcactcggttgcggatcgacactgccagcccaacaaagccggttgccgccgccgccaggatcgcgccgatgatgccggccacaccggccatcgcccaccaggtcgccgccttccggttccattcctgctggtactgcttcgcaatgctggacctcggctcaccataggctgaccgctcgatggcgtatgccgcttctccccttggcgtaaaacccagcgccgcaggcggcattgccatgctgcccgccgctttcccgaccacgacgcgcgcaccaggcttgcggtccagaccttcggccacggcgagctgcgcaaggacataatcagccgccgacttggctccacgcgcctcgatcagctcttgcactcgcgcgaaatccttggcctccacggccgccatgaatcgcgcacgcggcgaaggctccgcagggccg

SEQ ID NO: 14 - DNA, 14370 bp, Artificial Sequence
  Description of Artificial Sequence Synthetic plasmid
ccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcggccgcgggctggccgaggtgctgggcaagccctacctccaggcccccatcggggtcgagagcacgaccgccttcctgcgccgcctgggcgagattctgggcctcgatccggagcccttcatcgagcgcgagaagcactcgacgctgaagcccgtgtgggatctgtggcggagtgtcacgcaggacttcttcgggacggccaatttcggaatcgtggcgaccgaaacttatgcaagaggcatccgaaactatctcgaaggcgatctcgggctgccctgcgccttcgccgtggcccgcaagaggggctcgaagaccgacaacgaagcggtgcgcggactgatccgccagcaccgtccgctcgtgctcatggggtcgatcaacgagaagatttaccttgcggaactgaaagccggtcacggcccgcaaccctctttcatcgctgcctctttcccgggtgcggcgatccggcgcgctaccggaacgcccgttatgggatatgcaggtgctacgtggttactgcaggaagtttgcaacgccctgttcgacgccctgttccacattctgcccctcgggacggagatggacagcgccgccgccacaccgacgacactgcgccgcgacttcccgtgggatgccgatgcgcaagcggccctggaccgcatcgtagaggagcatccggttctcacccggatcagcgccgcgcgtgccttgcgcgacgccgccgagaaggctgccctcgatgccggtgccgagagggtcgtgagagagactgtcgaagccctgcgtgggccgggcttcggcgagaggaagggagagaaccaatgagcgatcatgccgtcaacacgccggtccatgccgccagggcccacgggcaccgagcaccacgtgccgagttctacgtctacttcgccgtcattctgctgggcgccttcccggtggccttcgtgagctggatcgtctcgacgatccgccaccgcaggcttcccaagcgcggccccttcgcgtccgcctggttcgatgccaaggcgatcacgccgctgattttccgcgcctgaccgcaggtcaggttgcgacacgccattcgtcgtctccccaaggggcggcggattaatcgggagggcatggtgccttaccgtaacccacgccaccagcatgtggaggatcgccatgcaccaccaccaccaccaccacgctagcgcgggcagatcttgaggccggccctccgtcgcgggcggcacccacgcccgcatcgattccaaggttcagccattgagacggctccgcttcgcgcgcaagcgcgggttgggccgactgcaagcggagagggaagcatggcactgctcagcttcgagcgaaaatatcgcgtgccggggggcacgctggtcggcggaaacctgttcgacttctgggtcggccctttctatgtcggcttcttcggggttgcgacgtttttcttcgcggccctgggtatcattctgattgcctggagtgccgtactccagggtacctggaacccccaactcatctctgtctacccgccggcccttgaatatggcctgggaggtgcacccctcgcaaaaggcgggctgtggcagatcatcacgatctgcgccactggtgccttcgtcagctgggcgctgcgcgaagtcgaaatctgccgtaagctgggcatcgggtaccacatcccgttcgccttcgcgttcgccatcctggcctacctgacgctggtgctgttccgcccggtgatgatgggcgcctggggctatgccttcccctacgggatctggacgcacctcgactgggtgtcgaacacgggctacacctacggcaacttccactacaaccctgcccacatgatcgccatctcgttcttcttcacgaacgcgctggctctggcgctgcacggcgcccttgtgctctccgcggccaaccccgagaagggcaaggaaatgcggacgccggatcacgaggatacgttcttccgcgatctggtcggctactcgatcgggacgctcggcatccaccgcctcggcctgctgctctcgctgagcgccgtcttcttcagcgccctctgcatgatcattaccggcaccatctggttcgatcagtgggtcgactggtggcaatggtgggtgaagctgccgtggtgggcgaacatcccgggaggcatcaatggctgagtatcagaacatcttctcccaggtccaggtccgcggaccggccgacctggggatgaccgaagacgtcaacctggccaaccgttcgggcgtcggtcccttctcgaccctgctcggctggttcggcaacgcccagctcggcccgatctatctcggctcgctcggcgtcctgtccctcttctcgggcctgatgtggttcttcaccatcgggatctggttctggtatcaggcgggctggaacccggccgtcttcctgcgcgacctgttcttcttctcgctcgagccgccggcacccgaatacggtctgtccttcgcggctccgctgaaggaaggcgggctgtggctgatcgcgtcgttcttcatgttcgtcgcggtctggtcctggtggggccgcacctatctccgcgctcaggcgctgggcatgggcaagcacaccgcctgggcgttcctctcggccatctggctgtggatggtgctgggcttcatccgtccgatcctcatggggtcctggtcggaagcggttccctacggcatcttctcgcacctcgactggacgaacaacttctcgctcgtccacggcaacctgttctacaaccccttccacggtctctcgatcgccttcctctacgggtcggccctgctcttcgcgatgcacggtgcgaccatcctcgcggtctcccgcttcggcggcgagcgcgagctggagcagatcgccgaccgcgggacggcagcggagcgggccgccctcttctggcgctggaccatgggtttcaacgccacgatggaaggcatccaccgctgggccatctggatggcggtcctcgtgaccctcaccggcggcatcgggatcctgctctcgggcacggtcgtggacaactggtacgtctggggccagaaccacggcatggcgccgctgaactgaggagcgatcacaatggctgacaagaccatcttcaacgatcacctcaacaccaatccgaagaccaaccttcgcctctgggtcgctttccagatgatgaagggtgcgggctgggctggcggcgtgttcttcgggacgctccttctcatcgggttcttccgggtggtcgggcggatgcttccgatccaggagaaccaggctccggcgccgaacatcaccggcgctctggagaccgggatcgagctgatcaagcatctcgtctgagacaagtctcggggcagggcggcgcgaggccgcccgctcctccaagtccgggccatatcgccggcccgggtccggggcgacaccacagcccggttcccttcctgttggcgacagggacctggtgccgtgtggaagaccgcacggcacccttttgacattcacgggaggctctgatgaccaatcccaccccgcgacccgaaaccccgcttttggatcgcgtctgctgcccggccgacatgaaggcgctgagtgacgccgaactggagcggctggccgacgaagtgcgttccgaggtcagtgataggggtagtttcttattttaggcagtttatatgaaattaagacatgcagatgtcacagtggatattgaactggtctcgaaagctcaatatcccccaaagcacaagcacaaacttcgacatcatgcagaagcgtttcccgaaccgcgtcttcgacgtgggcatcgccgagcagcatgccgtgaccttcgcggccggcctcgccggggccgggatgaagcccttctgcgcgatctattcctcgttcctgcaacggggttacgaccagatcgcccatgacgtggcgctgcagaaccttcccgtccgcttcgtgatcgaccgggcggggctcgtgggggccgatggcgcgacccatgcgggggccttcgacgttggcttcatcacttcgctgcccaacatgaccgtgatggccgcggccgacgaggccgagctcatccacatgatcgccaccgccgtggccttcgacgagggccccatcgccttccgcttcccgcggggcgagggggtgggcgtcgagatgcccgagcgcgggacggtgctggagcccggccggggccgcgtggtgcgcgaagggacggatgtcgcgatcctctccttcggcgcgcatctgcacgaggccttgcaggcggcgaaacttctcgaggccgagggggtgagcgtgaccgtggccgacgcccgcttctcgcgcccgctcgacacggggctcatcgaccagctcgtgcgccatcacgcggcgctggtaacggtggagcagggggccatgggcggcttcggcgcccatgtcatgcactatctcgccaattccggcggcttcgacgggggcctcgcgctccgggtcatgacgctgcccgaccgcttcatcgagcaggcgagccccgaggacatgtatgccgatgcggggctgcgggccgaggatatcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgcgttcaatcggaccagcggaggctggtccggaggccagacatgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccactgtcgactacgccatcatggcgacagcgcctttcctttgggttctctatatcgggcggatcgtggccggcatcaccggggcgactggggcggtagccggcgcttatattgccgatatcactgatggcgatgagcgcgcgcggcacttcggcttcatgagcgcctgtttcgggttcgggatggtcgcgggacctgtgctcggtgggctgatgggcggtttctccccccacgctccgttcttcgccgcggcagccttgaacggcctcaatttcctgacgggctgtttccttttgccggagtcgcacaaaggcgaacgccggccgttacgccgggaggctctcaacccgctcgcttcgttccggtgggcccggggcatgaccgtcgtcgccgccctgatggcggtcttcttcatcatgcaacttgtcggacaggtgccggccgcgctttgggtcattttcggcgaggatcgctttcactgggacgcgaccacgatcggcatttcgcttgccgcatttggcattctgcattcactcgcccaggcaatgatcaccggccctgtagccgcccggctcggcgaaaggcgggcactcatgctcggaatgattgccgacggcacaggctacatcctgcttgccttcgcgacacggggatggatggcgttcccgatcatggtcctgcttgcttcgggtggcatcggaatgccggcgctgcaagcaatgttgtccaggcaggtggatgaggaacgtcaggggcagctgcaaggctcactggcggcgctcaccagcctgacctcgatcgtcggacccctcctcttcacggcgatctatgcggcttctataacaacgtggaacgggtgggcatggattgcaggcgctgccctctacttgctctgcctgccggcgctgcgtcgcgggctttggagcggcgcagggcaacgagccgatcgctgatcgtggaaacgataggcctatgccatgcgggtcaaggcgacttccggcaagctatacgcgccctaggagtgcggttggaacgttggcccagccagatactcccgatcacgagcaggacgccgatgatttgaagcgcactcagcgtctgatccaagaacaaccatcctagcaacacggcggtccccgggctgagaaagcccagtaaggaaacaactgtaggttcgagtcgcgagatcccccggaaccaaaggaagtaggttaaacccgctccgatcaggccgagccacgccaggccgagaacattggttcctgtaggcatcgggattggcggatcaaacactaaagctactggaacgagcagaagtcctccggccgccagttgccaggcggtaaaggtgagcagaggcacgggaggttgccacttgcgggtcagcacggttccgaacgccatggaaaccgcccccgccaggcccgctgcgacgccgacaggatctagcgctgcgtttggtgtcaacaccaacagcgccacgcccgcagttccgcaaatagcccccaggaccgccatcaatcgtatcgggctacctagcagagcggcagagatgaacacgaccatcagcggctgcacagcgcctaccgtcgccgcgaccccgcccggcaggcggtagaccgaaataaacaacaagctccagaatagcgaaatattaagtgcgccgaggatgaagatgcgcatccaccagattcccgttggaatctgtcggacgatcatcacgagcaataaacccgccggcaacgcccgcagcagcataccggcgacccctcggcctcgctgttcgggctccacgaaaacgccggacagatgcgccttgtgagcgtccttggggccgtcctcctgtttgaagaccgacagcccaatgatctcgccgtcgatgtaggcgccgaatgccacggcatctcgcaaccgttcagcgaacgcctccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggtcgtggccgcgccaacctttgcgatccgcaagcgcgcggtcgccatcttcacgctggaacagtacgtcgaggcgggcatcatgacccgcgagcaatacgaggtcattaaaagcgccgtgattgatgatatagcggcccggctgctcctggttctcgcgcaccgaaatgggtgacttcaccccgcgctctttgatcgtggcaccgatttccgcgatgctctccggggaaaagccggggttgtcggccgtccgcggctgatgcggatcttcgtcgatcaggtccaggtccagctcgatagggccggaaccgccctgagacgccgcaggagcgtccaggaggctcgacaggtcgccgatgctatccaaccccaggccggacggctgcgccgcgcctgcggcttcctgagcggccgcagcggtgtttttcttggtggtcttggcttgagccgcagtcattgggaaatctccatcttcgtgaacacgtaatcagccagggcgcgaacctctttcgatgccttgcgcgcggccgttttcttgatcttccagaccggcacaccggatgcgagggcatcggcgatgctgctgcgcaggccaacggtggccggaatcatcatcttggggtacgcggccagcagctcggcttggtggcgcgcgtggcgcggattccgcgcatcgaccttgctgggcaccatgccaaggaattgcagcttggcgttcttctggcgcacgttcgcaatggtcgtgaccatcttcttgatgccctggatgctgtacgcctcaagctcgatgggggacagcacatagtcggccgcgaagagggcggccgccaggccgacgccaagggtcggggccgtgtcgatcaggcacacgtcgaagccttggttcgccagggccttgatgttcgccccgaacagctcgcgggcgtcgtccagcgacagccgttcggcgttcgccagtaccgggttggactcgatgagggcgaggcgcgcggcctggccgtcgccggctgcgggtgcggtttcggtccagccgccggcagggacagcgccgaacagcttgcttgcatgcaggccggtagcaaagtccttgagcgtgtaggacgcattgccctgggggtccaggtcgatcacggcaacccgcaagccgcgctcgaaaaagtcgaaggcaagatgcacaagggtcgaagtcttgccgacgccgcctttctggttggccgtgaccaaagttttcatcgtttggtttcctgttttttcttggcgtccgcttcccacttccggacgatgtacgcctgatgttccggcagaaccgccgttacccgcgcgtacccctcgggcaagttcttgtcctcgaacgcggcccacacgcgatgcaccgcttgcgacactgcgcccctggtcagtcccagcgacgttgcgaacgtcgcctgtggcttcccatcgactaagacgccccgcgctatctcgatggtctgctgccccacttccagcccctggatcgcctcctggaactggctttcggtaagccgtttcttcatggataacacccataatttgctccgcgccttggttgaacatagcggtgacagccgccagcacatgagagaagtttagctaaacatttctcgcacgtcaacacctttagccgctaaaactcgtccttggcgtaacaaaacaaaagcccggaaaccgggctttcgtctcttgccgcttatggctctgcacccggctccatcaccaacaggtcgcgcacgcgcttcactcggttgcggatcgacactgccagcccaacaaagccggttgccgccgccgccaggatcgcgccgatgatgccggccacaccggccatcgcccaccaggtcgccgccttccggttccattcctgctggtactgcttcgcaatgctggacctcggctcaccataggctgaccgctcgatggcgtatgccgcttctccccttggcgtaaaacccagcgccgcaggcggcattgccatgctgcccgccgctttcccgaccacgacgcgcgcaccaggcttgcggtccagaccttcggccacggcgagctgcgcaaggacataatcagccgccgacttggctccacgcgcctcgatcagctcttgcactcgcgcgaaatccttggcctccacggccgccatgaatcgcgcacgcggcgaaggctccgcagggccg

SEQ ID NO: 15 - DNA, 14398 bp, Artificial Sequence
  Description of Artificial Sequence Synthetic plasmid
ccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcggccgcgggctggccgaggtgctgggcaagccctacctccaggcccccatcggggtcgagagcacgaccgccttcctgcgccgcctgggcgagattctgggcctcgatccggagcccttcatcgagcgcgagaagcactcgacgctgaagcccgtgtgggatctgtggcggagtgtcacgcaggacttcttcgggacggccaatttcggaatcgtggcgaccgaaacttatgcaagaggcatccgaaactatctcgaaggcgatctcgggctgccctgcgccttcgccgtggcccgcaagaggggctcgaagaccgacaacgaagcggtgcgcggactgatccgccagcaccgtccgctcgtgctcatggggtcgatcaacgagaagatttaccttgcggaactgaaagccggtcacggcccgcaaccctctttcatcgctgcctctttcccgggtgcggcgatccggcgcgctaccggaacgcccgttatgggatatgcaggtgctacgtggttactgcaggaagtttgcaacgccctgttcgacgccctgttccacattctgcccctcgggacggagatggacagcgccgccgccacaccgacgacactgcgccgcgacttcccgtgggatgccgatgcgcaagcggccctggaccgcatcgtagaggagcatccggttctcacccggatcagcgccgcgcgtgccttgcgcgacgccgccgagaaggctgccctcgatgccggtgccgagagggtcgtgagagagactgtcgaagccctgcgtgggccgggcttcggcgagaggaagggagagaaccaatgagcgatcatgccgtcaacacgccggtccatgccgccagggcccacgggcaccgagcaccacgtgccgagttctacgtctacttcgccgtcattctgctgggcgccttcccggtggccttcgtgagctggatcgtctcgacgatccgccaccgcaggcttcccaagcgcggccccttcgcgtccgcctggttcgatgccaaggcgatcacgccgctgattttccgcgcctgaccgcaggtcaggttgcgacacgccattcgtcgtctccccaaggggcggcggattaatcgggagggcatggtgccttaccgtaacccacgccaccagcatgtggaggatcgccatgcaccaccaccaccaccaccacgagaacctgtacttccaatcctttacgtagaaatagggaagatcttgagggccggccctccgtcgcgggcggcacccacgcccgcatcgattccaaggttcagccattgagacggctccgcttcgcgcgcaagcgcgggttgggccgactgcaagcggagagggaagcatggcactgctcagcttcgagcgaaaatatcgcgtgccggggggcacgctggtcggcggaaacctgttcgacttctgggtcggccctttctatgtcggcttcttcggggttgcgacgtttttcttcgcggccctgggtatcattctgattgcctggagtgccgtactccagggtacctggaacccccaactcatctctgtctacccgccggcccttgaatatggcctgggaggtgcacccctcgcaaaaggcgggctgtggcagatcatcacgatctgcgccactggtgccttcgtcagctgggcgctgcgcgaagtcgaaatctgccgtaagctgggcatcgggtaccacatcccgttcgccttcgcgttcgccatcctggcctacctgacgctggtgctgttccgcccggtgatgatgggcgcctggggctatgccttcccctacgggatctggacgcacctcgactgggtgtcgaacacgggctacacctacggcaacttccactacaaccctgcccacatgatcgccatctcgttcttcttcacgaacgcgctggctctggcgctgcacggcgcccttgtgctctccgcggccaaccccgagaagggcaaggaaatgcggacgccggatcacgaggatacgttcttccgcgatctggtcggctactcgatcgggacgctcggcatccaccgcctcggcctgctgctctcgctgagcgccgtcttcttcagcgccctctgcatgatcattaccggcaccatctggttcgatcagtgggtcgactggtggcaatggtgggtgaagctgccgtggtgggcgaacatcccgggaggcatcaatggctgagtatcagaacatcttctcccaggtccaggtccgcggaccggccgacctggggatgaccgaagacgtcaacctggccaaccgttcgggcgtcggtcccttctcgaccctgctcggctggttcggcaacgcccagctcggcccgatctatctcggctcgctcggcgtcctgtccctcttctcgggcctgatgtggttcttcaccatcgggatctggttctggtatcaggcgggctggaacccggccgtcttcctgcgcgacctgttcttcttctcgctcgagccgccggcacccgaatacggtctgtccttcgcggctccgctgaaggaaggcgggctgtggctgatcgcgtcgttcttcatgttcgtcgcggtctggtcctggtggggccgcacctatctccgcgctcaggcgctgggcatgggcaagcacaccgcctgggcgttcctctcggccatctggctgtggatggtgctgggcttcatccgtccgatcctcatggggtcctggtcggaagcggttccctacggcatcttctcgcacctcgactggacgaacaacttctcgctcgtccacggcaacctgttctacaaccccttccacggtctctcgatcgccttcctctacgggtcggccctgctcttcgcgatgcacggtgcgaccatcctcgcggtctcccgcttcggcggcgagcgcgagctggagcagatcgccgaccgcgggacggcagcggagcgggccgccctcttctggcgctggaccatgggtttcaacgccacgatggaaggcatccaccgctgggccatctggatggcggtcctcgtgaccctcaccggcggcatcgggatcctgctctcgggcacggtcgtggacaactggtacgtctggggccagaaccacggcatggcgccgctgaactgaggagcgatcacaatggctgacaagaccatcttcaacgatcacctcaacaccaatccgaagaccaaccttcgcctctgggtcgctttccagatgatgaagggtgcgggctgggctggcggcgtgttcttcgggacgctccttctcatcgggttcttccgggtggtcgggcggatgcttccgatccaggagaaccaggctccggcgccgaacatcaccggcgctctggagaccgggatcgagctgatcaagcatctcgtctgagacaagtctcggggcagggcggcgcgaggccgcccgctcctccaagtccgggccatatcgccggcccgggtccggggcgacaccacagcccggttcccttcctgttggcgacagggacctggtgccgtgtggaagaccgcacggcacccttttgacattcacgggaggctctgatgaccaatcccaccccgcgacccgaaaccccgcttttggatcgcgtctgctgcccggccgacatgaaggcgctgagtgacgccgaactggagcggctggccgacgaagtgcgttccgaggtcagtgataggggtagtttcttattttaggcagtttatatgaaattaagacatgcagatgtcacagtggatattgaactggtctcgaaagctcaatatcccccaaagcacaagcacaaacttcgacatcatgcagaagcgtttcccgaaccgcgtcttcgacgtgggcatcgccgagcagcatgccgtgaccttcgcggccggcctcgccggggccgggatgaagcccttctgcgcgatctattcctcgttcctgcaacggggttacgaccagatcgcccatgacgtggcgctgcagaaccttcccgtccgcttcgtgatcgaccgggcggggctcgtgggggccgatggcgcgacccatgcgggggccttcgacgttggcttcatcacttcgctgcccaacatgaccgtgatggccgcggccgacgaggccgagctcatccacatgatcgccaccgccgtggccttcgacgagggccccatcgccttccgcttcccgcggggcgagggggtgggcgtcgagatgcccgagcgcgggacggtgctggagcccggccggggccgcgtggtgcgcgaagggacggatgtcgcgatcctctccttcggcgcgcatctgcacgaggccttgcaggcggcgaaacttctcgaggccgagggggtgagcgtgaccgtggccgacgcccgcttctcgcgcccgctcgacacggggctcatcgaccagctcgtgcgccatcacgcggcgctggtaacggtggagcagggggccatgggcggcttcggcgcccatgtcatgcactatctcgccaattccggcggcttcgacgggggcctcgcgctccgggtcatgacgctgcccgaccgcttcatcgagcaggcgagccccgaggacatgtatgccgatgcggggctgcgggccgaggatatcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgcgttcaatcggaccagcggaggctggtccggaggccagacatgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccactgtcgactacgccatcatggcgacagcgcctttcctttgggttctctatatcgggcggatcgtggccggcatcaccggggcgactggggcggtagccggcgcttatattgccgatatcactgatggcgatgagcgcgcgcggcacttcggcttcatgagcgcctgtttcgggttcgggatggtcgcgggacctgtgctcggtgggctgatgggcggtttctccccccacgctccgttcttcgccgcggcagccttgaacggcctcaatttcctgacgggctgtttccttttgccggagtcgcacaaaggcgaacgccggccgttacgccgggaggctctcaacccgctcgcttcgttccggtgggcccggggcatgaccgtcgtcgccgccctgatggcggtcttcttcatcatgcaacttgtcggacaggtgccggccgcgctttgggtcattttcggcgaggatcgctttcactgggacgcgaccacgatcggcatttcgcttgccgcatttggcattctgcattcactcgcccaggcaatgatcaccggccctgtagccgcccggctcggcgaaaggcgggcactcatgctcggaatgattgccgacggcacaggctacatcctgcttgccttcgcgacacggggatggatggcgttcccgatcatggtcctgcttgcttcgggtggcatcggaatgccggcgctgcaagcaatgttgtccaggcaggtggatgaggaacgtcaggggcagctgcaaggctcactggcggcgctcaccagcctgacctcgatcgtcggacccctcctcttcacggcgatctatgcggcttctataacaacgtggaacgggtgggcatggattgcaggcgctgccctctacttgctctgcctgccggcgctgcgtcgcgggctttggagcggcgcagggcaacgagccgatcgctgatcgtggaaacgataggcctatgccatgcgggtcaaggcgacttccggcaagctatacgcgccctaggagtgcggttggaacgttggcccagccagatactcccgatcacgagcaggacgccgatgatttgaagcgcactcagcgtctgatccaagaacaaccatcctagcaacacggcggtccccgggctgagaaagcccagtaaggaaacaactgtaggttcgagtcgcgagatcccccggaaccaaaggaagtaggttaaacccgctccgatcaggccgagccacgccaggccgagaacattggttcctgtaggcatcgggattggcggatcaaacactaaagctactggaacgagcagaagtcctccggccgccagttgccaggcggtaaaggtgagcagaggcacgggaggttgccacttgcgggtcagcacggttccgaacgccatggaaaccgcccccgccaggcccgctgcgacgccgacaggatctagcgctgcgtttggtgtcaacaccaacagcgccacgcccgcagttccgcaaatagcccccaggaccgccatcaatcgtatcgggctacctagcagagcggcagagatgaacacgaccatcagcggctgcacagcgcctaccgtcgccgcgaccccgcccggcaggcggtagaccgaaataaacaacaagctccagaatagcgaaatattaagtgcgccgaggatgaagatgcgcatccaccagattcccgttggaatctgtcggacgatcatcacgagcaataaacccgccggcaacgcccgcagcagcataccggcgacccctcggcctcgctgttcgggctccacgaaaacgccggacagatgcgccttgtgagcgtccttggggccgtcctcctgtttgaagaccgacagcccaatgatctcgccgtcgatgtaggcgccgaatgccacggcatctcgcaaccgttcagcgaacgcctccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggtcgtggccgcgccaacctttgcgatccgcaagcgcgcggtcgccatcttcacgctggaacagtacgtcgaggcgggcatcatgacccgcgagcaatacgaggtcattaaaagcgccgtgattgatgatatagcggcccggctgctcctggttctcgcgcaccgaaatgggtgacttcaccccgcgctctttgatcgtggcaccgatttccgcgatgctctccggggaaaagccggggttgtcggccgtccgcggctgatgcggatcttcgtcgatcaggtccaggtccagctcgatagggccggaaccgccctgagacgccgcaggagcgtccaggaggctcgacaggtcgccgatgctatccaaccccaggccggacggctgcgccgcgcctgcggcttcctgagcggccgcagcggtgtttttcttggtggtcttggcttgagccgcagtcattgggaaatctccatcttcgtgaacacgtaatcagccagggcgcgaacctctttcgatgccttgcgcgcggccgttttcttgatcttccagaccggcacaccggatgcgagggcatcggcgatgctgctgcgcaggccaacggtggccggaatcatcatcttggggtacgcggccagcagctcggcttggtggcgcgcgtggcgcggattccgcgcatcgaccttgctgggcaccatgccaaggaattgcagcttggcgttcttctggcgcacgttcgcaatggtcgtgaccatcttcttgatgccctggatgctgtacgcctcaagctcgatgggggacagcacatagtcggccgcgaagagggcggccgccaggccgacgccaagggtcggggccgtgtcgatcaggcacacgtcgaagccttggttcgccagggccttgatgttcgccccgaacagctcgcgggcgtcgtccagcgacagccgttcggcgttcgccagtaccgggttggactcgatgagggcgaggcgcgcggcctggccgtcgccggctgcgggtgcggtttcggtccagccgccggcagggacagcgccgaacagcttgcttgcatgcaggccggtagcaaagtccttgagcgtgtaggacgcattgccctgggggtccaggtcgatcacggcaacccgcaagccgcgctcgaaaaagtcgaaggcaagatgcacaagggtcgaagtcttgccgacgccgcctttctggttggccgtgaccaaagttttcatcgtttggtttcctgttttttcttggcgtccgcttcccacttccggacgatgtacgcctgatgttccggcagaaccgccgttacccgcgcgtacccctcgggcaagttcttgtcctcgaacgcggcccacacgcgatgcaccgcttgcgacactgcgcccctggtcagtcccagcgacgttgcgaacgtcgcctgtggcttcccatcgactaagacgccccgcgctatctcgatggtctgctgccccacttccagcccctggatcgcctcctggaactggctttcggtaagccgtttcttcatggataacacccataatttgctccgcgccttggttgaacatagcggtgacagccgccagcacatgagagaagtttagctaaacatttctcgcacgtcaacacctttagccgctaaaactcgtccttggcgtaacaaaacaaaagcccggaaaccgggctttcgtctcttgccgcttatggctctgcacccggctccatcaccaacaggtcgcgcacgcgcttcactcggttgcggatcgacactgccagcccaacaaagccggttgccgccgccgccaggatcgcgccgatgatgccggccacaccggccatcgcccaccaggtcgccgccttccggttccattcctgctggtactgcttcgcaatgctggacctcggctcaccataggctgaccgctcgatggcgtatgccgcttctccccttggcgtaaaacccagcgccgcaggcggcattgccatgctgcccgccgctttcccgaccacgacgcgcgcaccaggcttgcggtccagaccttcggccacggcgagctgcgcaaggacataatcagccgccgacttggctccacgcgcctcgatcagctcttgcactcgcgcgaaatccttggcctccacggccgccatgaatcgcgcacgcggcgaaggctccgcagggccg

SEQ ID NO: 16 - DNA, 14391 bp, Artificial Sequence
  Description of Artificial Sequence Synthetic plasmid
ccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcggccgcgggctggccgaggtgctgggcaagccctacctccaggcccccatcggggtcgagagcacgaccgccttcctgcgccgcctgggcgagattctgggcctcgatccggagcccttcatcgagcgcgagaagcactcgacgctgaagcccgtgtgggatctgtggcggagtgtcacgcaggacttcttcgggacggccaatttcggaatcgtggcgaccgaaacttatgcaagaggcatccgaaactatctcgaaggcgatctcgggctgccctgcgccttcgccgtggcccgcaagaggggctcgaagaccgacaacgaagcggtgcgcggactgatccgccagcaccgtccgctcgtgctcatggggtcgatcaacgagaagatttaccttgcggaactgaaagccggtcacggcccgcaaccctctttcatcgctgcctctttcccgggtgcggcgatccggcgcgctaccggaacgcccgttatgggatatgcaggtgctacgtggttactgcaggaagtttgcaacgccctgttcgacgccctgttccacattctgcccctcgggacggagatggacagcgccgccgccacaccgacgacactgcgccgcgacttcccgtgggatgccgatgcgcaagcggccctggaccgcatcgtagaggagcatccggttctcacccggatcagcgccgcgcgtgccttgcgcgacgccgccgagaaggctgccctcgatgccggtgccgagagggtcgtgagagagactgtcgaagccctgcgtgggccgggcttcggcgagaggaagggagagaaccaatgagcgatcatgccgtcaacacgccggtccatgccgccagggcccacgggcaccgagcaccacgtgccgagttctacgtctacttcgccgtcattctgctgggcgccttcccggtggccttcgtgagctggatcgtctcgacgatccgccaccgcaggcttcccaagcgcggccccttcgcgtccgcctggttcgatgccaaggcgatcacgccgctgattttccgcgcctgaccgcaggtcaggttgcgacacgccattcgtcgtctccccaaggggcggcggattaatcgggagggcatggtgccttaccgtaacccacgccaccagcatgtggaggatcgccatgcaccaccaccaccaccaccacgagaacctgtacttccagtccgctagctagggaagatcttgaggccggccctccgtcgcgggcggcacccacgcccgcatcgattccaaggttcagccattgagacggctccgcttcgcgcgcaagcgcgggttgggccgactgcaagcggagagggaagcatggcactgctcagcttcgagcgaaaatatcgcgtgccggggggcacgctggtcggcggaaacctgttcgacttctgggtcggccctttctatgtcggcttcttcggggttgcgacgtttttcttcgcggccctgggtatcattctgattgcctggagtgccgtactccagggtacctggaacccccaactcatctctgtctacccgccggcccttgaatatggcctgggaggtgcacccctcgcaaaaggcgggctgtggcagatcatcacgatctgcgccactggtgccttcgtcagctgggcgctgcgcgaagtcgaaatctgccgtaagctgggcatcgggtaccacatcccgttcgccttcgcgttcgccatcctggcctacctgacgctggtgctgttccgcccggtgatgatgggcgcctggggctatgccttcccctacgggatctggacgcacctcgactgggtgtcgaacacgggctacacctacggcaacttccactacaaccctgcccacatgatcgccatctcgttcttcttcacgaacgcgctggctctggcgctgcacggcgcccttgtgctctccgcggccaaccccgagaagggcaaggaaatgcggacgccggatcacgaggatacgttcttccgcgatctggtcggctactcgatcgggacgctcggcatccaccgcctcggcctgctgctctcgctgagcgccgtcttcttcagcgccctctgcatgatcattaccggcaccatctggttcgatcagtgggtcgactggtggcaatggtgggtgaagctgccgtggtgggcgaacatcccgggaggcatcaatggctgagtatcagaacatcttctcccaggtccaggtccgcggaccggccgacctggggatgaccgaagacgtcaacctggccaaccgttcgggcgtcggtcccttctcgaccctgctcggctggttcggcaacgcccagctcggcccgatctatctcggctcgctcggcgtcctgtccctcttctcgggcctgatgtggttcttcaccatcgggatctggttctggtatcaggcgggctggaacccggccgtcttcctgcgcgacctgttcttcttctcgctcgagccgccggcacccgaatacggtctgtccttcgcggctccgctgaaggaaggcgggctgtggctgatcgcgtcgttcttcatgttcgtcgcggtctggtcctggtggggccgcacctatctccgcgctcaggcgctgggcatgggcaagcacaccgcctgggcgttcctctcggccatctggctgtggatggtgctgggcttcatccgtccgatcctcatggggtcctggtcggaagcggttccctacggcatcttctcgcacctcgactggacgaacaacttctcgctcgtccacggcaacctgttctacaaccccttccacggtctctcgatcgccttcctctacgggtcggccctgctcttcgcgatgcacggtgcgaccatcctcgcggtctcccgcttcggcggcgagcgcgagctggagcagatcgccgaccgcgggacggcagcggagcgggccgccctcttctggcgctggaccatgggtttcaacgccacgatggaaggcatccaccgctgggccatctggatggcggtcctcgtgaccctcaccggcggcatcgggatcctgctctcgggcacggtcgtggacaactggtacgtctggggccagaaccacggcatggcgccgctgaactgaggagcgatcacaatggctgacaagaccatcttcaacgatcacctcaacaccaatccgaagaccaaccttcgcctctgggtcgctttccagatgatgaagggtgcgggctgggctggcggcgtgttcttcgggacgctccttctcatcgggttcttccgggtggtcgggcggatgcttccgatccaggagaaccaggctccggcgccgaacatcaccggcgctctggagaccgggatcgagctgatcaagcatctcgtctgagacaagtctcggggcagggcggcgcgaggccgcccgctcctccaagtccgggccatatcgccggcccgggtccggggcgacaccacagcccggttcccttcctgttggcgacagggacctggtgccgtgtggaagaccgcacggcacccttttgacattcacgggaggctctgatgaccaatcccaccccgcgacccgaaaccccgcttttggatcgcgtctgctgcccggccgacatgaaggcgctgagtgacgccgaactggagcggctggccgacgaagtgcgttccgaggtcagtgataggggtagtttcttattttaggcagtttatatgaaattaagacatgcagatgtcacagtggatattgaactggtctcgaaagctcaatatcccccaaagcacaagcacaaacttcgacatcatgcagaagcgtttcccgaaccgcgtcttcgacgtgggcatcgccgagcagcatgccgtgaccttcgcggccggcctcgccggggccgggatgaagcccttctgcgcgatctattcctcgttcctgcaacggggttacgaccagatcgcccatgacgtggcgctgcagaaccttcccgtccgcttcgtgatcgaccgggcggggctcgtgggggccgatggcgcgacccatgcgggggccttcgacgttggcttcatcacttcgctgcccaacatgaccgtgatggccgcggccgacgaggccgagctcatccacatgatcgccaccgccgtggccttcgacgagggccccatcgccttccgcttcccgcggggcgagggggtgggcgtcgagatgcccgagcgcgggacggtgctggagcccggccggggccgcgtggtgcgcgaagggacggatgtcgcgatcctctccttcggcgcgcatctgcacgaggccttgcaggcggcgaaacttctcgaggccgagggggtgagcgtgaccgtggccgacgcccgcttctcgcgcccgctcgacacggggctcatcgaccagctcgtgcgccatcacgcggcgctggtaacggtggagcagggggccatgggcggcttcggcgcccatgtcatgcactatctcgccaattccggcggcttcgacgggggcctcgcgctccgggtcatgacgctgcccgaccgcttcatcgagcaggcgagccccgaggacatgtatgccgatgcggggctgcgggccgaggatatcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgcgttcaatcggaccagcggaggctggtccggaggccagacatgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccactgtcgactacgccatcatggcgacagcgcctttcctttgggttctctatatcgggcggatcgtggccggcatcaccggggcgactggggcggtagccggcgcttatattgccgatatcactgatggcgatgagcgcgcgcggcacttcggcttcatgagcgcctgtttcgggttcgggatggtcgcgggacctgtgctcggtgggctgatgggcggtttctccccccacgctccgttcttcgccgcggcagccttgaacggcctcaatttcctgacgggctgtttccttttgccggagtcgcacaaaggcgaacgccggccgttacgccgggaggctctcaacccgctcgcttcgttccggtgggcccggggcatgaccgtcgtcgccgccctgatggcggtcttcttcatcatgcaacttgtcggacaggtgccggccgcgctttgggtcattttcggcgaggatcgctttcactgggacgcgaccacgatcggcatttcgcttgccgcatttggcattctgcattcactcgcccaggcaatgatcaccggccctgtagccgcccggctcggcgaaaggcgggcactcatgctcggaatgattgccgacggcacaggctacatcctgcttgccttcgcgacacggggatggatggcgttcccgatcatggtcctgcttgcttcgggtggcatcggaatgccggcgctgcaagcaatgttgtccaggcaggtggatgaggaacgtcaggggcagctgcaaggctcactggcggcgctcaccagcctgacctcgatcgtcggacccctcctcttcacggcgatctatgcggcttctataacaacgtggaacgggtgggcatggattgcaggcgctgccctctacttgctctgcctgccggcgctgcgtcgcgggctttggagcggcgcagggcaacgagccgatcgctgatcgtggaaacgataggcctatgccatgcgggtcaaggcgacttccggcaagctatacgcgccctaggagtgcggttggaacgttggcccagccagatactcccgatcacgagcaggacgccgatgatttgaagcgcactcagcgtctgatccaagaacaaccatcctagcaacacggcggtccccgggctgagaaagcccagtaaggaaacaactgtaggttcgagtcgcgagatcccccggaaccaaaggaagtaggttaaacccgctccgatcaggccgagccacgccaggccgagaacattggttcctgtaggcatcgggattggcggatcaaacactaaagctactggaacgagcagaagtcctccggccgccagttgccaggcggtaaaggtgagcagaggcacgggaggttgccacttgcgggtcagcacggttccgaacgccatggaaaccgcccccgccaggcccgctgcgacgccgacaggatctagcgctgcgtttggtgtcaacaccaacagcgccacgcccgcagttccgcaaatagcccccaggaccgccatcaatcgtatcgggctacctagcagagcggcagagatgaacacgaccatcagcggctgcacagcgcctaccgtcgccgcgaccccgcccggcaggcggtagaccgaaataaacaacaagctccagaatagcgaaatattaagtgcgccgaggatgaagatgcgcatccaccagattcccgttggaatctgtcggacgatcatcacgagcaataaacccgccggcaacgcccgcagcagcataccggcgacccctcggcctcgctgttcgggctccacgaaaacgccggacagatgcgccttgtgagcgtccttggggccgtcctcctgtttgaagaccgacagcccaatgatctcgccgtcgatgtaggcgccgaatgccacggcatctcgcaaccgttcagcgaacgcctccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggtcgtggccgcgccaacctttgcgatccgcaagcgcgcggtcgccatcttcacgctggaacagtacgtcgaggcgggcatcatgacccgcgagcaatacgaggtcattaaaagcgccgtgattgatgatatagcggcccggctgctcctggttctcgcgcaccgaaatgggtgacttcaccccgcgctctttgatcgtggcaccgatttccgcgatgctctccggggaaaagccggggttgtcggccgtccgcggctgatgcggatcttcgtcgatcaggtccaggtccagctcgatagggccggaaccgccctgagacgccgcaggagcgtccaggaggctcgacaggtcgccgatgctatccaaccccaggccggacggctgcgccgcgcctgcggcttcctgagcggccgcagcggtgtttttcttggtggtcttggcttgagccgcagtcattgggaaatctccatcttcgtgaacacgtaatcagccagggcgcgaacctctttcgatgccttgcgcgcggccgttttcttgatcttccagaccggcacaccggatgcgagggcatcggcgatgctgctgcgcaggccaacggtggccggaatcatcatcttggggtacgcggccagcagctcggcttggtggcgcgcgtggcgcggattccgcgcatcgaccttgctgggcaccatgccaaggaattgcagcttggcgttcttctggcgcacgttcgcaatggtcgtgaccatcttcttgatgccctggatgctgtacgcctcaagctcgatgggggacagcacatagtcggccgcgaagagggcggccgccaggccgacgccaagggtcggggccgtgtcgatcaggcacacgtcgaagccttggttcgccagggccttgatgttcgccccgaacagctcgcgggcgtcgtccagcgacagccgttcggcgttcgccagtaccgggttggactcgatgagggcgaggcgcgcggcctggccgtcgccggctgcgggtgcggtttcggtccagccgccggcagggacagcgccgaacagcttgcttgcatgcaggccggtagcaaagtccttgagcgtgtaggacgcattgccctgggggtccaggtcgatcacggcaacccgcaagccgcgctcgaaaaagtcgaaggcaagatgcacaagggtcgaagtcttgccgacgccgcctttctggttggccgtgaccaaagttttcatcgtttggtttcctgttttttcttggcgtccgcttcccacttccggacgatgtacgcctgatgttccggcagaaccgccgttacccgcgcgtacccctcgggcaagttcttgtcctcgaacgcggcccacacgcgatgcaccgcttgcgacactgcgcccctggtcagtcccagcgacgttgcgaacgtcgcctgtggcttcccatcgactaagacgccccgcgctatctcgatggtctgctgccccacttccagcccctggatcgcctcctggaactggctttcggtaagccgtttcttcatggataacacccataatttgctccgcgccttggttgaacatagcggtgacagccgccagcacatgagagaagtttagctaaacatttctcgcacgtcaacacctttagccgctaaaactcgtccttggcgtaacaaaacaaaagcccggaaaccgggctttcgtctcttgccgcttatggctctgcacccggctccatcaccaacaggtcgcgcacgcgcttcactcggttgcggatcgacactgccagcccaacaaagccggttgccgccgccgccaggatcgcgccgatgatgccggccacaccggccatcgcccaccaggtcgccgccttccggttccattcctgctggtactgcttcgcaatgctggacctcggctcaccataggctgaccgctcgatggcgtatgccgcttctccccttggcgtaaaacccagcgccgcaggcggcattgccatgctgcccgccgctttcccgaccacgacgcgcgcaccaggcttgcggtccagaccttcggccacggcgagctgcgcaaggacataatcagccgccgacttggctccacgcgcctcgatcagctcttgcactcgcgcgaaatccttggcctccacggccgccatgaatcgcgcacgcggcgaaggctccgcagggccg

SEQ ID NO: 17 - DNA, 14377 bp, Artificial Sequence
  Description of Artificial Sequence Synthetic plasmid
ccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcggccgcgggctggccgaggtgctgggcaagccctacctccaggcccccatcggggtcgagagcacgaccgccttcctgcgccgcctgggcgagattctgggcctcgatccggagcccttcatcgagcgcgagaagcactcgacgctgaagcccgtgtgggatctgtggcggagtgtcacgcaggacttcttcgggacggccaatttcggaatcgtggcgaccgaaacttatgcaagaggcatccgaaactatctcgaaggcgatctcgggctgccctgcgccttcgcctggcccgcaagaggggctcgaagaccgacaacgaagcggtgcgcggactgatccgccagcaccgtccgctcgtgctcatggggtcgatcaacgagaagatttaccttgcggaactgaaagccggtcacggcccggcaaccctctttcatcgctgcctctttcccgggtgcggcgatccggcgcgctaccggaacgcccgttatgggatatgcaggtgctacgtggttactgcaggaagtttgcaacgccctgttcgacgccctgttccacattctgcccctcgggacggagatggacagcgccgccgccacaccgacgacactgcgccgcgacttcccgtgggatgccgatgcgcaacgggccctggaccgcatcgtagaggagcatccggttctcacccggatcagcgccgcgcgtgccttgcgcgacgccgccgagaaggcttgccctcgatgccggtgccgagagggtcgtgagagagactgtcgaagccctgacgtgggccgggcttcggcgagaggaagggagagaaccaatgagcgatcatgccgtcaacacgccggtccatgccgccagggcccacgggcaccgagcacctcgtgccgagttctacgtctacttcgccgtcattctgctgggcgccttcccggtggccttcgtgagctggatcgtctcgacgatccgccaccgcaggcttcccaagcgcggccccttcgcgtccgcctggttcgatgccaaggcgatcacgccgctgattttccgcgcctgaccgcaggtcaggttgcgacacgccattcgtcgtctccccaaggggcggcggattaatcgggagggcatggtgccttaccgtaacccacgccaccagtaggcaggaggaacacgtgtcgtccggtggtcaccaccaccaccaccaccaccaccaccactaataggccggccctccgtcgcgggcggcacccacgcccgcatcgattccaaggttcagccattgagacggctccgcttcgcgcgcaagcgcgggttgggccgactgcaagcggagagggaagcatggcactgctcagcttcgagcgaaaatatcgcgtgccggggggcacgctggtcggcggaaacctgttcgacttctgggtcggccctttctatgtcggcttcttcggggttgcgacgtttttcttcgcggccctgggtatcattctgattgcctggagtgccgtactccagggtacctggaacccccaactcatctctgtctacccgccggcccttgaatatggcctgggaggtgcacccctcgcaaaaggcgggctgtggcagatcatcacgatctgcgccactggtgccttcgtcagctgggcgctgcgcgaagtcgaaatctgccgtaagctgggcatcgggtaccacatcccgttcgccttcgcgttcgccatcctggcctacctgacgctggtgctgttccgcccggtgatgatgggcgcctggggctatgccttcccctacgggatctggacgcacctcgactgggtgtcgaacacgggctacacctacggcaacttccactacaaccctgcccacatgatcgccatctcgttcttcttcacgaacgcgctggctctggcgctgcacggcgcccttgtgctctccgcggccaaccccgagaagggcaaggaaatgcggacgccggatcacgaggatacgttcttccgcgatctggtcggctactcgatcgggacgctcggcatccaccgcctcggcctgctgctctcgctgagcgccgtcttcttcagcgccctctgcatgatcattaccggcaccatctggttcgatcagtgggtcgactggtggcaatggtgggtgaagctgccgtggtgggcgaacatcccgggaggcatcaatggctgagtatcagaacatcttctcccaggtccaggtccgcggaccggccgacctggggatgaccgaagacgtcaacctggccaaccgttcgggcgtcggtcccttctcgaccctgctcggctggttcggcaacgcccagctcggcccgatctatctcggctcgctcggcgtcctgtccctcttctcgggcctgatgtggttcttcaccatcgggatctggttctggtatcaggcgggctggaacccggccgtcttcctgcgcgacctgttcttcttctcgctcgagccgccggcacccgaatacggtctgtccttcgcggctccgctgaaggaaggcgggctgtggctgatcgcgtcgttcttcatgttcgtcgcggtctggtcctggtggggccgcacctatctccgcgctcaggcgctgggcatgggcaagcacaccgcctgggcgttcctctcggccatctggctgtggatggtgctgggcttcatccgtccgatcctcatggggtcctggtcggaagcggttccctacggcatcttctcgcacctcgactggacgaacaacttctcgctcgtccacggcaacctgttctacaaccccttccacggtctctcgatcgccttcctctacgggtcggccctgctcttcgcgatgcacggtgcgaccatcctcgcggtctcccgcttcggcggcgagcgcgagctggagcagatcgccgaccgcgggacggcagcggagcgggccgccctcttctggcgctggaccatgggtttcaacgccacgatggaaggcatccaccgctgggccatctggatggcggtcctcgtgaccctcaccggcggcatcgggatcctgctctcgggcacggtcgtggacaactggtacgtctggggccagaaccacggcatggcgccgctgaactgaggagcgatcacaatggctgacaagaccatcttcaacgatcacctcaacaccaatccgaagaccaaccttcgcctctgggtcgctttccagatgatgaagggtgcgggctgggctggcggcgtgttcttcgggacgctccttctcatcgggttcttccgggtggtcgggcggatgcttccgatccaggagaaccaggctccggcgccgaacatcaccggcgctctggagaccgggatcgagctgatcaagcatctcgtctgagacaagtctcggggcagggcggcgcgaggccgcccgctcctccaagtccgggccatatcgccggcccgggtccggggcgacaccacagcccggttcccttcctgttggcgacagggacctggtgccgtgtggaagaccgcacggcacccttttgacattcacgggaggctctgatgaccaatcccaccccgcgacccgaaaccccgcttttggatcgcgtctgctgcccggccgacatgaaggcgctgagtgacgccgaactggagcggctggccgacgaagtgcgttccgaggtcagtgataggggtagtttcttattttaggcagtttatatgaaattaagacatgcagatgtcacagtggatattgaactggtctcgaaagctcaatatcccccaaagcacaagcacaaacttcgacatcatgcagaagcgtttcccgaaccgcgtcttcgacgtgggcatcgccgagcagcatgccgtgaccttcgcggccggcctcgccggggccgggatgaagcccttctgcgcgatctattcctcgttcctgcaacggggttacgaccagatcgcccatgacgtggcgctgcagaaccttcccgtccgcttcgtgatcgaccgggcggggctcgtgggggccgatggcgcgacccatgcgggggccttcgacgttggcttcatcacttcgctgcccaacatgaccgtgatggccgcggccgacgaggccgagctcatccacatgatcgccaccgccgtggccttcgacgagggccccatcgccttccgcttcccgcggggcgagggggtgggcgtcgagatgcccgagcgcgggacggtgctggagcccggccggggccgcgtggtgcgcgaagggacggatgtcgcgatcctctccttcggcgcgcatctgcacgaggccttgcaggcggcgaaacttctcgaggccgagggggtgagcgtgaccgtggccgacgcccgcttctcgcgcccgctcgacacggggctcatcgaccagctcgtgcgccatcacgcggcgctggtaacggtggagcagggggccatgggcggcttcggcgcccatgtcatgcactatctcgccaattccggcggcttcgacgggggcctcgcgctccgggtcatgacgctgcccgaccgcttcatcgagcaggcgagccccgaggacatgtatgccgatgcggggctgcgggccgaggatatcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgcgttcaatcggaccagcggaggctggtccggaggccagacatgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccactgtcgactacgccatcatggcgacagcgcctttcctttgggttctctatatcgggcggatcgtggccggcatcaccggggcgactggggcggtagccggcgcttatattgccgatatcactgatggcgatgagcgcgcgcggcacttcggcttcatgagcgcctgtttcgggttcgggatggtcgcgggacctgtgctcggtgggctgatgggcggtttctccccccacgctccgttcttcgccgcggcagccttgaacggcctcaatttcctgacgggctgtttccttttgccggagtcgcacaaaggcgaacgccggccgttacgccgggaggctctcaacccgctcgcttcgttccggtgggcccggggcatgaccgtcgtcgccgccctgatggcggtcttcttcatcatgcaacttgtcggacaggtgccggccgcgctttgggtcattttcggcgaggatcgctttcactgggacgcgaccacgatcggcatttcgcttgccgcatttggcattctgcattcactcgcccaggcaatgatcaccggccctgtagccgcccggctcggcgaaaggcgggcactcatgctcggaatgattgccgacggcacaggctacatcctgcttgccttcgcgacacggggatggatggcgttcccgatcatggtcctgcttgcttcgggtggcatcggaatgccggcgctgcaagcaatgttgtccaggcaggtggatgaggaacgtcaggggcagctgcaaggctcactggcggcgctcaccagcctgacctcgatcgtcggacccctcctcttcacggcgatctatgcggcttctataacaacgtggaacgggtgggcatggattgcaggcgctgccctctacttgctctgcctgccggcgctgcgtcgcgggctttggagcggcgcagggcaacgagccgatcgctgatcgtggaaacgataggcctatgccatgcgggtcaaggcgacttccggcaagctatacgcgccctaggagtgcggttggaacgttggcccagccagatactcccgatcacgagcaggacgccgatgatttgaagcgcactcagcgtctgatccaagaacaaccatcctagcaacacggcggtccccgggctgagaaagcccagtaaggaaacaactgtaggttcgagtcgcgagatcccccggaaccaaaggaagtaggttaaacccgctccgatcaggccgagccacgccaggccgagaacattggttcctgtaggcatcgggattggcggatcaaacactaaagctactggaacgagcagaagtcctccggccgccagttgccaggcggtaaaggtgagcagaggcacgggaggttgccacttgcgggtcagcacggttccgaacgccatggaaaccgcccccgccaggcccgctgcgacgccgacaggatctagcgctgcgtttggtgtcaacaccaacagcgccacgcccgcagttccgcaaatagcccccaggaccgccatcaatcgtatcgggctacctagcagagcggcagagatgaacacgaccatcagcggctgcacagcgcctaccgtcgccgcgaccccgcccggcaggcggtagaccgaaataaacaacaagctccagaatagcgaaatattaagtgcgccgaggatgaagatgcgcatccaccagattcccgttggaatctgtcggacgatcatcacgagcaataaacccgccggcaacgcccgcagcagcataccggcgacccctcggcctcgctgttcgggctccacgaaaacgccggacagatgcgccttgtgagcgtccttggggccgtcctcctgtttgaagaccgacagcccaatgatctcgccgtcgatgtaggcgccgaatgccacggcatctcgcaaccgttcagcgaacgcctccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggtcgtggccgcgccaacctttgcgatccgcaagcgcgcggtcgccatcttcacgctggaacagtacgtcgaggcgggcatcatgacccgcgagcaatacgaggtcattaaaagcgccgtgattgatgatatagcggcccggctgctcctggttctcgcgcaccgaaatgggtgacttcaccccgcgctctttgatcgtggcaccgatttccgcgatgctctccggggaaaagccggggttgtcggccgtccgcggctgatgcggatcttcgtcgatcaggtccaggtccagctcgatagggccggaaccgccctgagacgccgcaggagcgtccaggaggctcgacaggtcgccgatgctatccaaccccaggccggacggctgcgccgcgcctgcggcttcctgagcggccgcagcggtgtttttcttggtggtcttggcttgagccgcagtcattgggaaatctccatcttcgtgaacacgtaatcagccagggcgcgaacctctttcgatgccttgcgcgcggccgttttcttgatcttccagaccggcacaccggatgcgagggcatcggcgatgctgctgcgcaggccaacggtggccggaatcatcatcttggggtacgcggccagcagctcggcttggtggcgcgcgtggcgcggattccgcgcatcgaccttgctgggcaccatgccaaggaattgcagcttggcgttcttctggcgcacgttcgcaatggtcgtgaccatcttcttgatgccctggatgctgtacgcctcaagctcgatgggggacagcacatagtcggccgcgaagagggcggccgccaggccgacgccaagggtcggggccgtgtcgatcaggcacacgtcgaagccttggttcgccagggccttgatgttcgccccgaacagctcgcgggcgtcgtccagcgacagccgttcggcgttcgccagtaccgggttggactcgatgagggcgaggcgcgcggcctggccgtcgccggctgcgggtgcggtttcggtccagccgccggcagggacagcgccgaacagcttgcttgcatgcaggccggtagcaaagtccttgagcgtgtaggacgcattgccctgggggtccaggtcgatcacggcaacccgcaagccgcgctcgaaaaagtcgaaggcaagatgcacaagggtcgaagtcttgccgacgccgcctttctggttggccgtgaccaaagttttcatcgtttggtttcctgttttttcttggcgtccgcttcccacttccggacgatgtacgcctgatgttccggcagaaccgccgttacccgcgcgtacccctcgggcaagttcttgtcctcgaacgcggcccacacgcgatgcaccgcttgcgacactgcgcccctggtcagtcccagcgacgttgcgaacgtcgcctgtggcttcccatcgactaagacgccccgcgctatctcgatggtctgctgccccacttccagcccctggatcgcctcctggaactggctttcggtaagccgtttcttcatggataacacccataatttgctccgcgccttggttgaacatagcggtgacagccgccagcacatgagagaagtttagctaaacatttctcgcacgtcaacacctttagccgctaaaactcgtccttggcgtaacaaaacaaaagcccggaaaccgggctttcgtctcttgccgcttatggctctgcacccggctccatcaccaacaggtcgcgcacgcgcttcactcggttgcggatcgacactgccagcccaacaaagccggttgccgccgccgccaggatcgcgccgatgatgccggccacaccggccatcgcccaccaggtcgccgccttccggttccattcctgctggtactgcttcgcaatgctggacctcggctcaccataggctgaccgctcgatggcgtatgccgcttctccccttggcgtaaaacccagcgccgcaggcggcattgccatgctgcccgccgctttcccgaccacgacgcgcgcaccaggcttgcggtccagaccttcggccacggcgagctgcgcaaggacataatcagccgccgacttggctccacgcgcctcgatcagctcttgcactcgcgcgaaatccttggcctccacggccgccatgaatcgcgcacgcggcgaaggctccgcagggccg

SEQ ID NO: 18 - DNA, 14386 bp, Artificial Sequence
  Description of Artificial Sequence Synthetic plasmid
ccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcggccgcgggctggccgaggtgctgggcaagccctacctccaggcccccatcggggtcgagagcacgaccgccttcctgcgccgcctgggcgagattctgggcctcgatccggagcccttcatcgagcgcgagaagcactcgacgctgaagcccgtgtgggatctgtggcggagtgtcacgcaggacttcttcgggacggccaatttcggaatcgtggcgaccgaaacttatgcaagaggcatccgaaactatctcgaaggcgatctcgggctgccctgcgccttcgcctggcccgcaagaggggctcgaagaccgacaacgaagcggtgcgcggactgatccgccagcaccgtccgctcgtgctcatggggtcgatcaacgagaagatttaccttgcggaactgaaagccggtcacggcccggcaaccctctttcatcgctgcctctttcccgggtgcggcgatccggcgcgctaccggaacgcccgttatgggatatgcaggtgctacgtggttactgcaggaagtttgcaacgccctgttcgacgccctgttccacattctgcccctcgggacggagatggacagcgccgccgccacaccgacgacactgcgccgcgacttcccgtgggatgccgatgcgcaacgggccctggaccgcatcgtagaggagcatccggttctcacccggatcagcgccgcgcgtgccttgcgcgacgccgccgagaaggcttgccctcgatgccggtgccgagagggtcgtgagagagactgtcgaagccctgacgtgggccgggcttcggcgagaggaagggagagaaccaatgagcgatcatgccgtcaacacgccggtccatgccgccagggcccacgggcaccgagcacctcgtgccgagttctacgtctacttcgccgtcattctgctgggcgccttcccggtggccttcgtgagctggatcgtctcgacgatccgccaccgcaggcttcccaagcgcggccccttcgcgtccgcctggttcgatgccaaggcgatcacgccgctgattttccgcgcctgaccgcaggtcaggttgcgacacgccattcgtcgtctccccaaggggcggcggattaatcgggagggcatggtgccttaccgtaacccacgccaccagtaggcaggaggaacacgtgtcgtccggtggtcaccaccaccaccaccaccaccaccaccaccaccaccactaataggccggccctccgtcgcgggcggcacccacgcccgcatcgattccaaggttcagccattgagacggctccgcttcgcgcgcaagcgcgggttgggccgactgcaagcggagagggaagcatggcactgctcagcttcgagcgaaaatatcgcgtgccggggggcacgctggtcggcggaaacctgttcgacttctgggtcggccctttctatgtcggcttcttcggggttgcgacgtttttcttcgcggccctgggtatcattctgattgcctggagtgccgtactccagggtacctggaacccccaactcatctctgtctacccgccggcccttgaatatggcctgggaggtgcacccctcgcaaaaggcgggctgtggcagatcatcacgatctgcgccactggtgccttcgtcagctgggcgctgcgcgaagtcgaaatctgccgtaagctgggcatcgggtaccacatcccgttcgccttcgcgttcgccatcctggcctacctgacgctggtgctgttccgcccggtgatgatgggcgcctggggctatgccttcccctacgggatctggacgcacctcgactgggtgtcgaacacgggctacacctacggcaacttccactacaaccctgcccacatgatcgccatctcgttcttcttcacgaacgcgctggctctggcgctgcacggcgcccttgtgctctccgcggccaaccccgagaagggcaaggaaatgcggacgccggatcacgaggatacgttcttccgcgatctggtcggctactcgatcgggacgctcggcatccaccgcctcggcctgctgctctcgctgagcgccgtcttcttcagcgccctctgcatgatcattaccggcaccatctggttcgatcagtgggtcgactggtggcaatggtgggtgaagctgccgtggtgggcgaacatcccgggaggcatcaatggctgagtatcagaacatcttctcccaggtccaggtccgcggaccggccgacctggggatgaccgaagacgtcaacctggccaaccgttcgggcgtcggtcccttctcgaccctgctcggctggttcggcaacgcccagctcggcccgatctatctcggctcgctcggcgtcctgtccctcttctcgggcctgatgtggttcttcaccatcgggatctggttctggtatcaggcgggctggaacccggccgtcttcctgcgcgacctgttcttcttctcgctcgagccgccggcacccgaatacggtctgtccttcgcggctccgctgaaggaaggcgggctgtggctgatcgcgtcgttcttcatgttcgtcgcggtctggtcctggtggggccgcacctatctccgcgctcaggcgctgggcatgggcaagcacaccgcctgggcgttcctctcggccatctggctgtggatggtgctgggcttcatccgtccgatcctcatggggtcctggtcggaagcggttccctacggcatcttctcgcacctcgactggacgaacaacttctcgctcgtccacggcaacctgttctacaaccccttccacggtctctcgatcgccttcctctacgggtcggccctgctcttcgcgatgcacggtgcgaccatcctcgcggtctcccgcttcggcggcgagcgcgagctggagcagatcgccgaccgcgggacggcagcggagcgggccgccctcttctggcgctggaccatgggtttcaacgccacgatggaaggcatccaccgctgggccatctggatggcggtcctcgtgaccctcaccggcggcatcgggatcctgctctcgggcacggtcgtggacaactggtacgtctggggccagaaccacggcatggcgccgctgaactgaggagcgatcacaatggctgacaagaccatcttcaacgatcacctcaacaccaatccgaagaccaaccttcgcctctgggtcgctttccagatgatgaagggtgcgggctgggctggcggcgtgttcttcgggacgctccttctcatcgggttcttccgggtggtcgggcggatgcttccgatccaggagaaccaggctccggcgccgaacatcaccggcgctctggagaccgggatcgagctgatcaagcatctcgtctgagacaagtctcggggcagggcggcgcgaggccgcccgctcctccaagtccgggccatatcgccggcccgggtccggggcgacaccacagcccggttcccttcctgttggcgacagggacctggtgccgtgtggaagaccgcacggcacccttttgacattcacgggaggctctgatgaccaatcccaccccgcgacccgaaaccccgcttttggatcgcgtctgctgcccggccgacatgaaggcgctgagtgacgccgaactggagcggctggccgacgaagtgcgttccgaggtcagtgataggggtagtttcttattttaggcagtttatatgaaattaagacatgcagatgtcacagtggatattgaactggtctcgaaagctcaatatcccccaaagcacaagcacaaacttcgacatcatgcagaagcgtttcccgaaccgcgtcttcgacgtgggcatcgccgagcagcatgccgtgaccttcgcggccggcctcgccggggccgggatgaagcccttctgcgcgatctattcctcgttcctgcaacggggttacgaccagatcgcccatgacgtggcgctgcagaaccttcccgtccgcttcgtgatcgaccgggcggggctcgtgggggccgatggcgcgacccatgcgggggccttcgacgttggcttcatcacttcgctgcccaacatgaccgtgatggccgcggccgacgaggccgagctcatccacatgatcgccaccgccgtggccttcgacgagggccccatcgccttccgcttcccgcggggcgagggggtgggcgtcgagatgcccgagcgcgggacggtgctggagcccggccggggccgcgtggtgcgcgaagggacggatgtcgcgatcctctccttcggcgcgcatctgcacgaggccttgcaggcggcgaaacttctcgaggccgagggggtgagcgtgaccgtggccgacgcccgcttctcgcgcccgctcgacacggggctcatcgaccagctcgtgcgccatcacgcggcgctggtaacggtggagcagggggccatgggcggcttcggcgcccatgtcatgcactatctcgccaattccggcggcttcgacgggggcctcgcgctccgggtcatgacgctgcccgaccgcttcatcgagcaggcgagccccgaggacatgtatgccgatgcggggctgcgggccgaggatatcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgcgttcaatcggaccagcggaggctggtccggaggccagacatgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccactgtcgactacgccatcatggcgacagcgcctttcctttgggttctctatatcgggcggatcgtggccggcatcaccggggcgactggggcggtagccggcgcttatattgccgatatcactgatggcgatgagcgcgcgcggcacttcggcttcatgagcgcctgtttcgggttcgggatggtcgcgggacctgtgctcggtgggctgatgggcggtttctccccccacgctccgttcttcgccgcggcagccttgaacggcctcaatttcctgacgggctgtttccttttgccggagtcgcacaaaggcgaacgccggccgttacgccgggaggctctcaacccgctcgcttcgttccggtgggcccggggcatgaccgtcgtcgccgccctgatggcggtcttcttcatcatgcaacttgtcggacaggtgccggccgcgctttgggtcattttcggcgaggatcgctttcactgggacgcgaccacgatcggcatttcgcttgccgcatttggcattctgcattcactcgcccaggcaatgatcaccggccctgtagccgcccggctcggcgaaaggcgggcactcatgctcggaatgattgccgacggcacaggctacatcctgcttgccttcgcgacacggggatggatggcgttcccgatcatggtcctgcttgcttcgggtggcatcggaatgccggcgctgcaagcaatgttgtccaggcaggtggatgaggaacgtcaggggcagctgcaaggctcactggcggcgctcaccagcctgacctcgatcgtcggacccctcctcttcacggcgatctatgcggcttctataacaacgtggaacgggtgggcatggattgcaggcgctgccctctacttgctctgcctgccggcgctgcgtcgcgggctttggagcggcgcagggcaacgagccgatcgctgatcgtggaaacgataggcctatgccatgcgggtcaaggcgacttccggcaagctatacgcgccctaggagtgcggttggaacgttggcccagccagatactcccgatcacgagcaggacgccgatgatttgaagcgcactcagcgtctgatccaagaacaaccatcctagcaacacggcggtccccgggctgagaaagcccagtaaggaaacaactgtaggttcgagtcgcgagatcccccggaaccaaaggaagtaggttaaacccgctccgatcaggccgagccacgccaggccgagaacattggttcctgtaggcatcgggattggcggatcaaacactaaagctactggaacgagcagaagtcctccggccgccagttgccaggcggtaaaggtgagcagaggcacgggaggttgccacttgcgggtcagcacggttccgaacgccatggaaaccgcccccgccaggcccgctgcgacgccgacaggatctagcgctgcgtttggtgtcaacaccaacagcgccacgcccgcagttccgcaaatagcccccaggaccgccatcaatcgtatcgggctacctagcagagcggcagagatgaacacgaccatcagcggctgcacagcgcctaccgtcgccgcgaccccgcccggcaggcggtagaccgaaataaacaacaagctccagaatagcgaaatattaagtgcgccgaggatgaagatgcgcatccaccagattcccgttggaatctgtcggacgatcatcacgagcaataaacccgccggcaacgcccgcagcagcataccggcgacccctcggcctcgctgttcgggctccacgaaaacgccggacagatgcgccttgtgagcgtccttggggccgtcctcctgtttgaagaccgacagcccaatgatctcgccgtcgatgtaggcgccgaatgccacggcatctcgcaaccgttcagcgaacgcctccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggtcgtggccgcgccaacctttgcgatccgcaagcgcgcggtcgccatcttcacgctggaacagtacgtcgaggcgggcatcatgacccgcgagcaatacgaggtcattaaaagcgccgtgattgatgatatagcggcccggctgctcctggttctcgcgcaccgaaatgggtgacttcaccccgcgctctttgatcgtggcaccgatttccgcgatgctctccggggaaaagccggggttgtcggccgtccgcggctgatgcggatcttcgtcgatcaggtccaggtccagctcgatagggccggaaccgccctgagacgccgcaggagcgtccaggaggctcgacaggtcgccgatgctatccaaccccaggccggacggctgcgccgcgcctgcggcttcctgagcggccgcagcggtgtttttcttggtggtcttggcttgagccgcagtcattgggaaatctccatcttcgtgaacacgtaatcagccagggcgcgaacctctttcgatgccttgcgcgcggccgttttcttgatcttccagaccggcacaccggatgcgagggcatcggcgatgctgctgcgcaggccaacggtggccggaatcatcatcttggggtacgcggccagcagctcggcttggtggcgcgcgtggcgcggattccgcgcatcgaccttgctgggcaccatgccaaggaattgcagcttggcgttcttctggcgcacgttcgcaatggtcgtgaccatcttcttgatgccctggatgctgtacgcctcaagctcgatgggggacagcacatagtcggccgcgaagagggcggccgccaggccgacgccaagggtcggggccgtgtcgatcaggcacacgtcgaagccttggttcgccagggccttgatgttcgccccgaacagctcgcgggcgtcgtccagcgacagccgttcggcgttcgccagtaccgggttggactcgatgagggcgaggcgcgcggcctggccgtcgccggctgcgggtgcggtttcggtccagccgccggcagggacagcgccgaacagcttgcttgcatgcaggccggtagcaaagtccttgagcgtgtaggacgcattgccctgggggtccaggtcgatcacggcaacccgcaagccgcgctcgaaaaagtcgaaggcaagatgcacaagggtcgaagtcttgccgacgccgcctttctggttggccgtgaccaaagttttcatcgtttggtttcctgttttttcttggcgtccgcttcccacttccggacgatgtacgcctgatgttccggcagaaccgccgttacccgcgcgtacccctcgggcaagttcttgtcctcgaacgcggcccacacgcgatgcaccgcttgcgacactgcgcccctggtcagtcccagcgacgttgcgaacgtcgcctgtggcttcccatcgactaagacgccccgcgctatctcgatggtctgctgccccacttccagcccctggatcgcctcctggaactggctttcggtaagccgtttcttcatggataacacccataatttgctccgcgccttggttgaacatagcggtgacagccgccagcacatgagagaagtttagctaaacatttctcgcacgtcaacacctttagccgctaaaactcgtccttggcgtaacaaaacaaaagcccggaaaccgggctttcgtctcttgccgcttatggctctgcacccggctccatcaccaacaggtcgcgcacgcgcttcactcggttgcggatcgacactgccagcccaacaaagccggttgccgccgccgccaggatcgcgccgatgatgccggccacaccggccatcgcccaccaggtcgccgccttccggttccattcctgctggtactgcttcgcaatgctggacctcggctcaccataggctgaccgctcgatggcgtatgccgcttctccccttggcgtaaaacccagcgccgcaggcggcattgccatgctgcccgccgctttcccgaccacgacgcgcgcaccaggcttgcggtccagaccttcggccacggcgagctgcgcaaggacataatcagccgccgacttggctccacgcgcctcgatcagctcttgcactcgcgcgaaatccttggcctccacggccgccatgaatcgcgcacgcggcgaaggctccgcagggccg

SEQ ID NO: 19 - DNA, 14368 bp, Artificial Sequence
  Description of Artificial Sequence Synthetic plasmid
ccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcggccgcgggctggccgaggtgctgggcaagccctacctccaggcccccatcggggtcgagagcacgaccgccttcctgcgccgcctgggcgagattctgggcctcgatccggagcccttcatcgagcgcgagaagcactcgacgctgaagcccgtgtgggatctgtggcggagtgtcacgcaggacttcttcgggacggccaatttcggaatcgtggcgaccgaaacttatgcaagaggcatccgaaactatctcgaaggcgatctcgggctgccctgcgccttcgcctggcccgcaagaggggctcgaagaccgacaacgaagcggtgcgcggactgatccgccagcaccgtccgctcgtgctcatggggtcgatcaacgagaagatttaccttgcggaactgaaagccggtcacggcccggcaaccctctttcatcgctgcctctttcccgggtgcggcgatccggcgcgctaccggaacgcccgttatgggatatgcaggtgctacgtggttactgcaggaagtttgcaacgccctgttcgacgccctgttccacattctgcccctcgggacggagatggacagcgccgccgccacaccgacgacactgcgccgcgacttcccgtgggatgccgatgcgcaacgggccctggaccgcatcgtagaggagcatccggttctcacccggatcagcgccgcgcgtgccttgcgcgacgccgccgagaaggcttgccctcgatgccggtgccgagagggtcgtgagagagactgtcgaagccctgacgtgggccgggcttcggcgagaggaagggagagaaccaatgagcgatcatgccgtcaacacgccggtccatgccgccagggcccacgggcaccgagcacctcgtgccgagttctacgtctacttcgccgtcattctgctgggcgccttcccggtggccttcgtgagctggatcgtctcgacgatccgccaccgcaggcttcccaagcgcggccccttcgcgtccgcctggttcgatgccaaggcgatcacgccgctgattttccgcgcctgaccgcaggtcaggttgcgacacgccattcgtcgtctccccaaggggcggcggattaatcgggagggcatggtgccttaccgtaacccacgccaccagtaggcaggaggaacacgtgtcgtccggtggtcaccaccaccaccaccaccactaataggccggccctccgtcgcgggcggcacccacgcccgcatcgattccaaggttcagccattgagacggctccgcttcgcgcgcaagcgcgggttgggccgactgcaagcggagagggaagcatggcactgctcagcttcgagcgaaaatatcgcgtgccggggggcacgctggtcggcggaaacctgttcgacttctgggtcggccctttctatgtcggcttcttcggggttgcgacgtttttcttcgcggccctgggtatcattctgattgcctggagtgccgtactccagggtacctggaacccccaactcatctctgtctacccgccggcccttgaatatggcctgggaggtgcacccctcgcaaaaggcgggctgtggcagatcatcacgatctgcgccactggtgccttcgtcagctgggcgctgcgcgaagtcgaaatctgccgtaagctgggcatcgggtaccacatcccgttcgccttcgcgttcgccatcctggcctacctgacgctggtgctgttccgcccggtgatgatgggcgcctggggctatgccttcccctacgggatctggacgcacctcgactgggtgtcgaacacgggctacacctacggcaacttccactacaaccctgcccacatgatcgccatctcgttcttcttcacgaacgcgctggctctggcgctgcacggcgcccttgtgctctccgcggccaaccccgagaagggcaaggaaatgcggacgccggatcacgaggatacgttcttccgcgatctggtcggctactcgatcgggacgctcggcatccaccgcctcggcctgctgctctcgctgagcgccgtcttcttcagcgccctctgcatgatcattaccggcaccatctggttcgatcagtgggtcgactggtggcaatggtgggtgaagctgccgtggtgggcgaacatcccgggaggcatcaatggctgagtatcagaacatcttctcccaggtccaggtccgcggaccggccgacctggggatgaccgaagacgtcaacctggccaaccgttcgggcgtcggtcccttctcgaccctgctcggctggttcggcaacgcccagctcggcccgatctatctcggctcgctcggcgtcctgtccctcttctcgggcctgatgtggttcttcaccatcgggatctggttctggtatcaggcgggctggaacccggccgtcttcctgcgcgacctgttcttcttctcgctcgagccgccggcacccgaatacggtctgtccttcgcggctccgctgaaggaaggcgggctgtggctgatcgcgtcgttcttcatgttcgtcgcggtctggtcctggtggggccgcacctatctccgcgctcaggcgctgggcatgggcaagcacaccgcctgggcgttcctctcggccatctggctgtggatggtgctgggcttcatccgtccgatcctcatggggtcctggtcggaagcggttccctacggcatcttctcgcacctcgactggacgaacaacttctcgctcgtccacggcaacctgttctacaaccccttccacggtctctcgatcgccttcctctacgggtcggccctgctcttcgcgatgcacggtgcgaccatcctcgcggtctcccgcttcggcggcgagcgcgagctggagcagatcgccgaccgcgggacggcagcggagcgggccgccctcttctggcgctggaccatgggtttcaacgccacgatggaaggcatccaccgctgggccatctggatggcggtcctcgtgaccctcaccggcggcatcgggatcctgctctcgggcacggtcgtggacaactggtacgtctggggccagaaccacggcatggcgccgctgaactgaggagcgatcacaatggctgacaagaccatcttcaacgatcacctcaacaccaatccgaagaccaaccttcgcctctgggtcgctttccagatgatgaagggtgcgggctgggctggcggcgtgttcttcgggacgctccttctcatcgggttcttccgggtggtcgggcggatgcttccgatccaggagaaccaggctccggcgccgaacatcaccggcgctctggagaccgggatcgagctgatcaagcatctcgtctgagacaagtctcggggcagggcggcgcgaggccgcccgctcctccaagtccgggccatatcgccggcccgggtccggggcgacaccacagcccggttcccttcctgttggcgacagggacctggtgccgtgtggaagaccgcacggcacccttttgacattcacgggaggctctgatgaccaatcccaccccgcgacccgaaaccccgcttttggatcgcgtctgctgcccggccgacatgaaggcgctgagtgacgccgaactggagcggctggccgacgaagtgcgttccgaggtcagtgataggggtagtttcttattttaggcagtttatatgaaattaagacatgcagatgtcacagtggatattgaactggtctcgaaagctcaatatcccccaaagcacaagcacaaacttcgacatcatgcagaagcgtttcccgaaccgcgtcttcgacgtgggcatcgccgagcagcatgccgtgaccttcgcggccggcctcgccggggccgggatgaagcccttctgcgcgatctattcctcgttcctgcaacggggttacgaccagatcgcccatgacgtggcgctgcagaaccttcccgtccgcttcgtgatcgaccgggcggggctcgtgggggccgatggcgcgacccatgcgggggccttcgacgttggcttcatcacttcgctgcccaacatgaccgtgatggccgcggccgacgaggccgagctcatccacatgatcgccaccgccgtggccttcgacgagggccccatcgccttccgcttcccgcggggcgagggggtgggcgtcgagatgcccgagcgcgggacggtgctggagcccggccggggccgcgtggtgcgcgaagggacggatgtcgcgatcctctccttcggcgcgcatctgcacgaggccttgcaggcggcgaaacttctcgaggccgagggggtgagcgtgaccgtggccgacgcccgcttctcgcgcccgctcgacacggggctcatcgaccagctcgtgcgccatcacgcggcgctggtaacggtggagcagggggccatgggcggcttcggcgcccatgtcatgcactatctcgccaattccggcggcttcgacgggggcctcgcgctccgggtcatgacgctgcccgaccgcttcatcgagcaggcgagccccgaggacatgtatgccgatgcggggctgcgggccgaggatatcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgcgttcaatcggaccagcggaggctggtccggaggccagacatgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccactgtcgactacgccatcatggcgacagcgcctttcctttgggttctctatatcgggcggatcgtggccggcatcaccggggcgactggggcggtagccggcgcttatattgccgatatcactgatggcgatgagcgcgcgcggcacttcggcttcatgagcgcctgtttcgggttcgggatggtcgcgggacctgtgctcggtgggctgatgggcggtttctccccccacgctccgttcttcgccgcggcagccttgaacggcctcaatttcctgacgggctgtttccttttgccggagtcgcacaaaggcgaacgccggccgttacgccgggaggctctcaacccgctcgcttcgttccggtgggcccggggcatgaccgtcgtcgccgccctgatggcggtcttcttcatcatgcaacttgtcggacaggtgccggccgcgctttgggtcattttcggcgaggatcgctttcactgggacgcgaccacgatcggcatttcgcttgccgcatttggcattctgcattcactcgcccaggcaatgatcaccggccctgtagccgcccggctcggcgaaaggcgggcactcatgctcggaatgattgccgacggcacaggctacatcctgcttgccttcgcgacacggggatggatggcgttcccgatcatggtcctgcttgcttcgggtggcatcggaatgccggcgctgcaagcaatgttgtccaggcaggtggatgaggaacgtcaggggcagctgcaaggctcactggcggcgctcaccagcctgacctcgatcgtcggacccctcctcttcacggcgatctatgcggcttctataacaacgtggaacgggtgggcatggattgcaggcgctgccctctacttgctctgcctgccggcgctgcgtcgcgggctttggagcggcgcagggcaacgagccgatcgctgatcgtggaaacgataggcctatgccatgcgggtcaaggcgacttccggcaagctatacgcgccctaggagtgcggttggaacgttggcccagccagatactcccgatcacgagcaggacgccgatgatttgaagcgcactcagcgtctgatccaagaacaaccatcctagcaacacggcggtccccgggctgagaaagcccagtaaggaaacaactgtaggttcgagtcgcgagatcccccggaaccaaaggaagtaggttaaacccgctccgatcaggccgagccacgccaggccgagaacattggttcctgtaggcatcgggattggcggatcaaacactaaagctactggaacgagcagaagtcctccggccgccagttgccaggcggtaaaggtgagcagaggcacgggaggttgccacttgcgggtcagcacggttccgaacgccatggaaaccgcccccgccaggcccgctgcgacgccgacaggatctagcgctgcgtttggtgtcaacaccaacagcgccacgcccgcagttccgcaaatagcccccaggaccgccatcaatcgtatcgggctacctagcagagcggcagagatgaacacgaccatcagcggctgcacagcgcctaccgtcgccgcgaccccgcccggcaggcggtagaccgaaataaacaacaagctccagaatagcgaaatattaagtgcgccgaggatgaagatgcgcatccaccagattcccgttggaatctgtcggacgatcatcacgagcaataaacccgccggcaacgcccgcagcagcataccggcgacccctcggcctcgctgttcgggctccacgaaaacgccggacagatgcgccttgtgagcgtccttggggccgtcctcctgtttgaagaccgacagcccaatgatctcgccgtcgatgtaggcgccgaatgccacggcatctcgcaaccgttcagcgaacgcctccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggtcgtggccgcgccaacctttgcgatccgcaagcgcgcggtcgccatcttcacgctggaacagtacgtcgaggcgggcatcatgacccgcgagcaatacgaggtcattaaaagcgccgtgattgatgatatagcggcccggctgctcctggttctcgcgcaccgaaatgggtgacttcaccccgcgctctttgatcgtggcaccgatttccgcgatgctctccggggaaaagccggggttgtcggccgtccgcggctgatgcggatcttcgtcgatcaggtccaggtccagctcgatagggccggaaccgccctgagacgccgcaggagcgtccaggaggctcgacaggtcgccgatgctatccaaccccaggccggacggctgcgccgcgcctgcggcttcctgagcggccgcagcggtgtttttcttggtggtcttggcttgagccgcagtcattgggaaatctccatcttcgtgaacacgtaatcagccagggcgcgaacctctttcgatgccttgcgcgcggccgttttcttgatcttccagaccggcacaccggatgcgagggcatcggcgatgctgctgcgcaggccaacggtggccggaatcatcatcttggggtacgcggccagcagctcggcttggtggcgcgcgtggcgcggattccgcgcatcgaccttgctgggcaccatgccaaggaattgcagcttggcgttcttctggcgcacgttcgcaatggtcgtgaccatcttcttgatgccctggatgctgtacgcctcaagctcgatgggggacagcacatagtcggccgcgaagagggcggccgccaggccgacgccaagggtcggggccgtgtcgatcaggcacacgtcgaagccttggttcgccagggccttgatgttcgccccgaacagctcgcgggcgtcgtccagcgacagccgttcggcgttcgccagtaccgggttggactcgatgagggcgaggcgcgcggcctggccgtcgccggctgcgggtgcggtttcggtccagccgccggcagggacagcgccgaacagcttgcttgcatgcaggccggtagcaaagtccttgagcgtgtaggacgcattgccctgggggtccaggtcgatcacggcaacccgcaagccgcgctcgaaaaagtcgaaggcaagatgcacaagggtcgaagtcttgccgacgccgcctttctggttggccgtgaccaaagttttcatcgtttggtttcctgttttttcttggcgtccgcttcccacttccggacgatgtacgcctgatgttccggcagaaccgccgttacccgcgcgtacccctcgggcaagttcttgtcctcgaacgcggcccacacgcgatgcaccgcttgcgacactgcgcccctggtcagtcccagcgacgttgcgaacgtcgcctgtggcttcccatcgactaagacgccccgcgctatctcgatggtctgctgccccacttccagcccctggatcgcctcctggaactggctttcggtaagccgtttcttcatggataacacccataatttgctccgcgccttggttgaacatagcggtgacagccgccagcacatgagagaagtttagctaaacatttctcgcacgtcaacacctttagccgctaaaactcgtccttggcgtaacaaaacaaaagcccggaaaccgggctttcgtctcttgccgcttatggctctgcacccggctccatcaccaacaggtcgcgcacgcgcttcactcggttgcggatcgacactgccagcccaacaaagccggttgccgccgccgccaggatcgcgccgatgatgccggccacaccggccatcgcccaccaggtcgccgccttccggttccattcctgctggtactgcttcgcaatgctggacctcggctcaccataggctgaccgctcgatggcgtatgccgcttctccccttggcgtaaaacccagcgccgcaggcggcattgccatgctgcccgccgctttcccgaccacgacgcgcgcaccaggcttgcggtccagaccttcggccacggcgagctgcgcaaggacataatcagccgccgacttggctccacgcgcctcgatcagctcttgcactcgcgcgaaatccttggcctccacggccgccatgaatcgcgcacgcggcgaaggctccgcagggccg

SEQ ID NO: 20 - DNA, 14375 bp, Artificial Sequence
  Description of Artificial Sequence Synthetic plasmid
ccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcggccgcgggctggccgaggtgctgggcaagccctacctccaggcccccatcggggtcgagagcacgaccgccttcctgcgccgcctgggcgagattctgggcctcgatccggagcccttcatcgagcgcgagaagcactcgacgctgaagcccgtgtgggatctgtggcggagtgtcacgcaggacttcttcgggacggccaatttcggaatcgtggcgaccgaaacttatgcaagaggcatccgaaactatctcgaaggcgatctcgggctgccctgcgccttcgccgtggcccgcaagaggggctcgaagaccgacaacgaagcggtgcgcggactgatccgccagcaccgtccgctcgtgctcatggggtcgatcaacgagaagatttaccttgcggaactgaaagccggtcacggcccgcaaccctctttcatcgctgcctctttcccgggtgcggcgatccggcgcgctaccggaacgcccgttatgggatatgcaggtgctacgtggttactgcaggaagtttgcaacgccctgttcgacgccctgttccacattctgcccctcgggacggagatggacagcgccgccgccacaccgacgacactgcgccgcgacttcccgtgggatgccgatgcgcaagcggccctggaccgcatcgtagaggagcatccggttctcacccggatcagcgccgcgcgtgccttgcgcgacgccgccgagaaggctgccctcgatgccggtgccgagagggtcgtgagagagactgtcgaagccctgcgtgggccgggcttcggcgagaggaagggagagaaccaatgagcgatcatgccgtcaacacgccggtccatgccgccagggcccacgggcaccgagcaccacgtgccgagttctacgtctacttcgccgtcattctgctgggcgccttcccggtggccttcgtgagctggatcgtctcgacgatccgccaccgcaggcttcccaagcgcggccccttcgcgtccgcctggttcgatgccaaggcgatcacgccgctgattttccgcgcctgaccgcaggtcaggttgcgacacgccattcgtcgtctccccaaggggcggcggattaatcgggagggcatggtgccttaccgtaacccacgccaccagcatgtggcgactagttccatatgatagatctcaccaccaccaccaccaccaccaccaccactaataggccggccctccgtcgcgggcggcacccacgcccgcatcgattccaaggttcagccattgagacggctccgcttcgcgcgcaagcgcgggttgggccgactgcaagcggagagggaagcatggcactgctcagcttcgagcgaaaatatcgcgtgccggggggcacgctggtcggcggaaacctgttcgacttctgggtcggccctttctatgtcggcttcttcggggttgcgacgtttttcttcgcggccctgggtatcattctgattgcctggagtgccgtactccagggtacctggaacccccaactcatctctgtctacccgccggcccttgaatatggcctgggaggtgcacccctcgcaaaaggcgggctgtggcagatcatcacgatctgcgccactggtgccttcgtcagctgggcgctgcgcgaagtcgaaatctgccgtaagctgggcatcgggtaccacatcccgttcgccttcgcgttcgccatcctggcctacctgacgctggtgctgttccgcccggtgatgatgggcgcctggggctatgccttcccctacgggatctggacgcacctcgactgggtgtcgaacacgggctacacctacggcaacttccactacaaccctgcccacatgatcgccatctcgttcttcttcacgaacgcgctggctctggcgctgcacggcgcccttgtgctctccgcggccaaccccgagaagggcaaggaaatgcggacgccggatcacgaggatacgttcttccgcgatctggtcggctactcgatcgggacgctcggcatccaccgcctcggcctgctgctctcgctgagcgccgtcttcttcagcgccctctgcatgatcattaccggcaccatctggttcgatcagtgggtcgactggtggcaatggtgggtgaagctgccgtggtgggcgaacatcccgggaggcatcaatggctgagtatcagaacatcttctcccaggtccaggtccgcggaccggccgacctggggatgaccgaagacgtcaacctggccaaccgttcgggcgtcggtcccttctcgaccctgctcggctggttcggcaacgcccagctcggcccgatctatctcggctcgctcggcgtcctgtccctcttctcgggcctgatgtggttcttcaccatcgggatctggttctggtatcaggcgggctggaacccggccgtcttcctgcgcgacctgttcttcttctcgctcgagccgccggcacccgaatacggtctgtccttcgcggctccgctgaaggaaggcgggctgtggctgatcgcgtcgttcttcatgttcgtcgcggtctggtcctggtggggccgcacctatctccgcgctcaggcgctgggcatgggcaagcacaccgcctgggcgttcctctcggccatctggctgtggatggtgctgggcttcatccgtccgatcctcatggggtcctggtcggaagcggttccctacggcatcttctcgcacctcgactggacgaacaacttctcgctcgtccacggcaacctgttctacaaccccttccacggtctctcgatcgccttcctctacgggtcggccctgctcttcgcgatgcacggtgcgaccatcctcgcggtctcccgcttcggcggcgagcgcgagctggagcagatcgccgaccgcgggacggcagcggagcgggccgccctcttctggcgctggaccatgggtttcaacgccacgatggaaggcatccaccgctgggccatctggatggcggtcctcgtgaccctcaccggcggcatcgggatcctgctctcgggcacggtcgtggacaactggtacgtctggggccagaaccacggcatggcgccgctgaactgaggagcgatcacaatggctgacaagaccatcttcaacgatcacctcaacaccaatccgaagaccaaccttcgcctctgggtcgctttccagatgatgaagggtgcgggctgggctggcggcgtgttcttcgggacgctccttctcatcgggttcttccgggtggtcgggcggatgcttccgatccaggagaaccaggctccggcgccgaacatcaccggcgctctggagaccgggatcgagctgatcaagcatctcgtctgagacaagtctcggggcagggcggcgcgaggccgcccgctcctccaagtccgggccatatcgccggcccgggtccggggcgacaccacagcccggttcccttcctgttggcgacagggacctggtgccgtgtggaagaccgcacggcacccttttgacattcacgggaggctctgatgaccaatcccaccccgcgacccgaaaccccgcttttggatcgcgtctgctgcccggccgacatgaaggcgctgagtgacgccgaactggagcggctggccgacgaagtgcgttccgaggtcagtgataggggtagtttcttattttaggcagtttatatgaaattaagacatgcagatgtcacagtggatattgaactggtctcgaaagctcaatatcccccaaagcacaagcacaaacttcgacatcatgcagaagcgtttcccgaaccgcgtcttcgacgtgggcatcgccgagcagcatgccgtgaccttcgcggccggcctcgccggggccgggatgaagcccttctgcgcgatctattcctcgttcctgcaacggggttacgaccagatcgcccatgacgtggcgctgcagaaccttcccgtccgcttcgtgatcgaccgggcggggctcgtgggggccgatggcgcgacccatgcgggggccttcgacgttggcttcatcacttcgctgcccaacatgaccgtgatggccgcggccgacgaggccgagctcatccacatgatcgccaccgccgtggccttcgacgagggccccatcgccttccgcttcccgcggggcgagggggtgggcgtcgagatgcccgagcgcgggacggtgctggagcccggccggggccgcgtggtgcgcgaagggacggatgtcgcgatcctctccttcggcgcgcatctgcacgaggccttgcaggcggcgaaacttctcgaggccgagggggtgagcgtgaccgtggccgacgcccgcttctcgcgcccgctcgacacggggctcatcgaccagctcgtgcgccatcacgcggcgctggtaacggtggagcagggggccatgggcggcttcggcgcccatgtcatgcactatctcgccaattccggcggcttcgacgggggcctcgcgctccgggtcatgacgctgcccgaccgcttcatcgagcaggcgagccccgaggacatgtatgccgatgcggggctgcgggccgaggatatcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgcgttcaatcggaccagcggaggctggtccggaggccagacatgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccactgtcgactacgccatcatggcgacagcgcctttcctttgggttctctatatcgggcggatcgtggccggcatcaccggggcgactggggcggtagccggcgcttatattgccgatatcactgatggcgatgagcgcgcgcggcacttcggcttcatgagcgcctgtttcgggttcgggatggtcgcgggacctgtgctcggtgggctgatgggcggtttctccccccacgctccgttcttcgccgcggcagccttgaacggcctcaatttcctgacgggctgtttccttttgccggagtcgcacaaaggcgaacgccggccgttacgccgggaggctctcaacccgctcgcttcgttccggtgggcccggggcatgaccgtcgtcgccgccctgatggcggtcttcttcatcatgcaacttgtcggacaggtgccggccgcgctttgggtcattttcggcgaggatcgctttcactgggacgcgaccacgatcggcatttcgcttgccgcatttggcattctgcattcactcgcccaggcaatgatcaccggccctgtagccgcccggctcggcgaaaggcgggcactcatgctcggaatgattgccgacggcacaggctacatcctgcttgccttcgcgacacggggatggatggcgttcccgatcatggtcctgcttgcttcgggtggcatcggaatgccggcgctgcaagcaatgttgtccaggcaggtggatgaggaacgtcaggggcagctgcaaggctcactggcggcgctcaccagcctgacctcgatcgtcggacccctcctcttcacggcgatctatgcggcttctataacaacgtggaacgggtgggcatggattgcaggcgctgccctctacttgctctgcctgccggcgctgcgtcgcgggctttggagcggcgcagggcaacgagccgatcgctgatcgtggaaacgataggcctatgccatgcgggtcaaggcgacttccggcaagctatacgcgccctaggagtgcggttggaacgttggcccagccagatactcccgatcacgagcaggacgccgatgatttgaagcgcactcagcgtctgatccaagaacaaccatcctagcaacacggcggtccccgggctgagaaagcccagtaaggaaacaactgtaggttcgagtcgcgagatcccccggaaccaaaggaagtaggttaaacccgctccgatcaggccgagccacgccaggccgagaacattggttcctgtaggcatcgggattggcggatcaaacactaaagctactggaacgagcagaagtcctccggccgccagttgccaggcggtaaaggtgagcagaggcacgggaggttgccacttgcgggtcagcacggttccgaacgccatggaaaccgcccccgccaggcccgctgcgacgccgacaggatctagcgctgcgtttggtgtcaacaccaacagcgccacgcccgcagttccgcaaatagcccccaggaccgccatcaatcgtatcgggctacctagcagagcggcagagatgaacacgaccatcagcggctgcacagcgcctaccgtcgccgcgaccccgcccggcaggcggtagaccgaaataaacaacaagctccagaatagcgaaatattaagtgcgccgaggatgaagatgcgcatccaccagattcccgttggaatctgtcggacgatcatcacgagcaataaacccgccggcaacgcccgcagcagcataccggcgacccctcggcctcgctgttcgggctccacgaaaacgccggacagatgcgccttgtgagcgtccttggggccgtcctcctgtttgaagaccgacagcccaatgatctcgccgtcgatgtaggcgccgaatgccacggcatctcgcaaccgttcagcgaacgcctccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggtcgtggccgcgccaacctttgcgatccgcaagcgcgcggtcgccatcttcacgctggaacagtacgtcgaggcgggcatcatgacccgcgagcaatacgaggtcattaaaagcgccgtgattgatgatatagcggcccggctgctcctggttctcgcgcaccgaaatgggtgacttcaccccgcgctctttgatcgtggcaccgatttccgcgatgctctccggggaaaagccggggttgtcggccgtccgcggctgatgcggatcttcgtcgatcaggtccaggtccagctcgatagggccggaaccgccctgagacgccgcaggagcgtccaggaggctcgacaggtcgccgatgctatccaaccccaggccggacggctgcgccgcgcctgcggcttcctgagcggccgcagcggtgtttttcttggtggtcttggcttgagccgcagtcattgggaaatctccatcttcgtgaacacgtaatcagccagggcgcgaacctctttcgatgccttgcgcgcggccgttttcttgatcttccagaccggcacaccggatgcgagggcatcggcgatgctgctgcgcaggccaacggtggccggaatcatcatcttggggtacgcggccagcagctcggcttggtggcgcgcgtggcgcggattccgcgcatcgaccttgctgggcaccatgccaaggaattgcagcttggcgttcttctggcgcacgttcgcaatggtcgtgaccatcttcttgatgccctggatgctgtacgcctcaagctcgatgggggacagcacatagtcggccgcgaagagggcggccgccaggccgacgccaagggtcggggccgtgtcgatcaggcacacgtcgaagccttggttcgccagggccttgatgttcgccccgaacagctcgcgggcgtcgtccagcgacagccgttcggcgttcgccagtaccgggttggactcgatgagggcgaggcgcgcggcctggccgtcgccggctgcgggtgcggtttcggtccagccgccggcagggacagcgccgaacagcttgcttgcatgcaggccggtagcaaagtccttgagcgtgtaggacgcattgccctgggggtccaggtcgatcacggcaacccgcaagccgcgctcgaaaaagtcgaaggcaagatgcacaagggtcgaagtcttgccgacgccgcctttctggttggccgtgaccaaagttttcatcgtttggtttcctgttttttcttggcgtccgcttcccacttccggacgatgtacgcctgatgttccggcagaaccgccgttacccgcgcgtacccctcgggcaagttcttgtcctcgaacgcggcccacacgcgatgcaccgcttgcgacactgcgcccctggtcagtcccagcgacgttgcgaacgtcgcctgtggcttcccatcgactaagacgccccgcgctatctcgatggtctgctgccccacttccagcccctggatcgcctcctggaactggctttcggtaagccgtttcttcatggataacacccataatttgctccgcgccttggttgaacatagcggtgacagccgccagcacatgagagaagtttagctaaacatttctcgcacgtcaacacctttagccgctaaaactcgtccttggcgtaacaaaacaaaagcccggaaaccgggctttcgtctcttgccgcttatggctctgcacccggctccatcaccaacaggtcgcgcacgcgcttcactcggttgcggatcgacactgccagcccaacaaagccggttgccgccgccgccaggatcgcgccgatgatgccggccacaccggccatcgcccaccaggtcgccgccttccggttccattcctgctggtactgcttcgcaatgctggacctcggctcaccataggctgaccgctcgatggcgtatgccgcttctccccttggcgtaaaacccagcgccgcaggcggcattgccatgctgcccgccgctttcccgaccacgacgcgcgcaccaggcttgcggtccagaccttcggccacggcgagctgcgcaaggacataatcagccgccgacttggctccacgcgcctcgatcagctcttgcactcgcgcgaaatccttggcctccacggccgccatgaatcgcgcacgcggcgaaggctccgcagggccg

SEQ ID NO: 21 - DNA, 14384 bp, Artificial Sequence
  Description of Artificial Sequence Synthetic plasmid
ccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcggccgcgggctggccgaggtgctgggcaagccctacctccaggcccccatcggggtcgagagcacgaccgccttcctgcgccgcctgggcgagattctgggcctcgatccggagcccttcatcgagcgcgagaagcactcgacgctgaagcccgtgtgggatctgtggcggagtgtcacgcaggacttcttcgggacggccaatttcggaatcgtggcgaccgaaacttatgcaagaggcatccgaaactatctcgaaggcgatctcgggctgccctgcgccttcgccgtggcccgcaagaggggctcgaagaccgacaacgaagcggtgcgcggactgatccgccagcaccgtccgctcgtgctcatggggtcgatcaacgagaagatttaccttgcggaactgaaagccggtcacggcccgcaaccctctttcatcgctgcctctttcccgggtgcggcgatccggcgcgctaccggaacgcccgttatgggatatgcaggtgctacgtggttactgcaggaagtttgcaacgccctgttcgacgccctgttccacattctgcccctcgggacggagatggacagcgccgccgccacaccgacgacactgcgccgcgacttcccgtgggatgccgatgcgcaagcggccctggaccgcatcgtagaggagcatccggttctcacccggatcagcgccgcgcgtgccttgcgcgacgccgccgagaaggctgccctcgatgccggtgccgagagggtcgtgagagagactgtcgaagccctgcgtgggccgggcttcggcgagaggaagggagagaaccaatgagcgatcatgccgtcaacacgccggtccatgccgccagggcccacgggcaccgagcaccacgtgccgagttctacgtctacttcgccgtcattctgctgggcgccttcccggtggccttcgtgagctggatcgtctcgacgatccgccaccgcaggcttcccaagcgcggccccttcgcgtccgcctggttcgatgccaaggcgatcacgccgctgattttccgcgcctgaccgcaggtcaggttgcgacacgccattcgtcgtctccccaaggggcggcggattaatcgggagggcatggtgccttaccgtaacccacgccaccagcatgtggcgactagttccatatgatagatctcaccaccaccaccaccaccaccaccaccaccaccaccactaataggccggccctccgtcgcgggcggcacccacgcccgcatcgattccaaggttcagccattgagacggctccgcttcgcgcgcaagcgcgggttgggccgactgcaagcggagagggaagcatggcactgctcagcttcgagcgaaaatatcgcgtgccggggggcacgctggtcggcggaaacctgttcgacttctgggtcggccctttctatgtcggcttcttcggggttgcgacgtttttcttcgcggccctgggtatcattctgattgcctggagtgccgtactccagggtacctggaacccccaactcatctctgtctacccgccggcccttgaatatggcctgggaggtgcacccctcgcaaaaggcgggctgtggcagatcatcacgatctgcgccactggtgccttcgtcagctgggcgctgcgcgaagtcgaaatctgccgtaagctgggcatcgggtaccacatcccgttcgccttcgcgttcgccatcctggcctacctgacgctggtgctgttccgcccggtgatgatgggcgcctggggctatgccttcccctacgggatctggacgcacctcgactgggtgtcgaacacgggctacacctacggcaacttccactacaaccctgcccacatgatcgccatctcgttcttcttcacgaacgcgctggctctggcgctgcacggcgcccttgtgctctccgcggccaaccccgagaagggcaaggaaatgcggacgccggatcacgaggatacgttcttccgcgatctggtcggctactcgatcgggacgctcggcatccaccgcctcggcctgctgctctcgctgagcgccgtcttcttcagcgccctctgcatgatcattaccggcaccatctggttcgatcagtgggtcgactggtggcaatggtgggtgaagctgccgtggtgggcgaacatcccgggaggcatcaatggctgagtatcagaacatcttctcccaggtccaggtccgcggaccggccgacctggggatgaccgaagacgtcaacctggccaaccgttcgggcgtcggtcccttctcgaccctgctcggctggttcggcaacgcccagctcggcccgatctatctcggctcgctcggcgtcctgtccctcttctcgggcctgatgtggttcttcaccatcgggatctggttctggtatcaggcgggctggaacccggccgtcttcctgcgcgacctgttcttcttctcgctcgagccgccggcacccgaatacggtctgtccttcgcggctccgctgaaggaaggcgggctgtggctgatcgcgtcgttcttcatgttcgtcgcggtctggtcctggtggggccgcacctatctccgcgctcaggcgctgggcatgggcaagcacaccgcctgggcgttcctctcggccatctggctgtggatggtgctgggcttcatccgtccgatcctcatggggtcctggtcggaagcggttccctacggcatcttctcgcacctcgactggacgaacaacttctcgctcgtccacggcaacctgttctacaaccccttccacggtctctcgatcgccttcctctacgggtcggccctgctcttcgcgatgcacggtgcgaccatcctcgcggtctcccgcttcggcggcgagcgcgagctggagcagatcgccgaccgcgggacggcagcggagcgggccgccctcttctggcgctggaccatgggtttcaacgccacgatggaaggcatccaccgctgggccatctggatggcggtcctcgtgaccctcaccggcggcatcgggatcctgctctcgggcacggtcgtggacaactggtacgtctggggccagaaccacggcatggcgccgctgaactgaggagcgatcacaatggctgacaagaccatcttcaacgatcacctcaacaccaatccgaagaccaaccttcgcctctgggtcgctttccagatgatgaagggtgcgggctgggctggcggcgtgttcttcgggacgctccttctcatcgggttcttccgggtggtcgggcggatgcttccgatccaggagaaccaggctccggcgccgaacatcaccggcgctctggagaccgggatcgagctgatcaagcatctcgtctgagacaagtctcggggcagggcggcgcgaggccgcccgctcctccaagtccgggccatatcgccggcccgggtccggggcgacaccacagcccggttcccttcctgttggcgacagggacctggtgccgtgtggaagaccgcacggcacccttttgacattcacgggaggctctgatgaccaatcccaccccgcgacccgaaaccccgcttttggatcgcgtctgctgcccggccgacatgaaggcgctgagtgacgccgaactggagcggctggccgacgaagtgcgttccgaggtcagtgataggggtagtttcttattttaggcagtttatatgaaattaagacatgcagatgtcacagtggatattgaactggtctcgaaagctcaatatcccccaaagcacaagcacaaacttcgacatcatgcagaagcgtttcccgaaccgcgtcttcgacgtgggcatcgccgagcagcatgccgtgaccttcgcggccggcctcgccggggccgggatgaagcccttctgcgcgatctattcctcgttcctgcaacggggttacgaccagatcgcccatgacgtggcgctgcagaaccttcccgtccgcttcgtgatcgaccgggcggggctcgtgggggccgatggcgcgacccatgcgggggccttcgacgttggcttcatcacttcgctgcccaacatgaccgtgatggccgcggccgacgaggccgagctcatccacatgatcgccaccgccgtggccttcgacgagggccccatcgccttccgcttcccgcggggcgagggggtgggcgtcgagatgcccgagcgcgggacggtgctggagcccggccggggccgcgtggtgcgcgaagggacggatgtcgcgatcctctccttcggcgcgcatctgcacgaggccttgcaggcggcgaaacttctcgaggccgagggggtgagcgtgaccgtggccgacgcccgcttctcgcgcccgctcgacacggggctcatcgaccagctcgtgcgccatcacgcggcgctggtaacggtggagcagggggccatgggcggcttcggcgcccatgtcatgcactatctcgccaattccggcggcttcgacgggggcctcgcgctccgggtcatgacgctgcccgaccgcttcatcgagcaggcgagccccgaggacatgtatgccgatgcggggctgcgggccgaggatatcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgcgttcaatcggaccagcggaggctggtccggaggccagacatgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccactgtcgactacgccatcatggcgacagcgcctttcctttgggttctctatatcgggcggatcgtggccggcatcaccggggcgactggggcggtagccggcgcttatattgccgatatcactgatggcgatgagcgcgcgcggcacttcggcttcatgagcgcctgtttcgggttcgggatggtcgcgggacctgtgctcggtgggctgatgggcggtttctccccccacgctccgttcttcgccgcggcagccttgaacggcctcaatttcctgacgggctgtttccttttgccggagtcgcacaaaggcgaacgccggccgttacgccgggaggctctcaacccgctcgcttcgttccggtgggcccggggcatgaccgtcgtcgccgccctgatggcggtcttcttcatcatgcaacttgtcggacaggtgccggccgcgctttgggtcattttcggcgaggatcgctttcactgggacgcgaccacgatcggcatttcgcttgccgcatttggcattctgcattcactcgcccaggcaatgatcaccggccctgtagccgcccggctcggcgaaaggcgggcactcatgctcggaatgattgccgacggcacaggctacatcctgcttgccttcgcgacacggggatggatggcgttcccgatcatggtcctgcttgcttcgggtggcatcggaatgccggcgctgcaagcaatgttgtccaggcaggtggatgaggaacgtcaggggcagctgcaaggctcactggcggcgctcaccagcctgacctcgatcgtcggacccctcctcttcacggcgatctatgcggcttctataacaacgtggaacgggtgggcatggattgcaggcgctgccctctacttgctctgcctgccggcgctgcgtcgcgggctttggagcggcgcagggcaacgagccgatcgctgatcgtggaaacgataggcctatgccatgcgggtcaaggcgacttccggcaagctatacgcgccctaggagtgcggttggaacgttggcccagccagatactcccgatcacgagcaggacgccgatgatttgaagcgcactcagcgtctgatccaagaacaaccatcctagcaacacggcggtccccgggctgagaaagcccagtaaggaaacaactgtaggttcgagtcgcgagatcccccggaaccaaaggaagtaggttaaacccgctccgatcaggccgagccacgccaggccgagaacattggttcctgtaggcatcgggattggcggatcaaacactaaagctactggaacgagcagaagtcctccggccgccagttgccaggcggtaaaggtgagcagaggcacgggaggttgccacttgcgggtcagcacggttccgaacgccatggaaaccgcccccgccaggcccgctgcgacgccgacaggatctagcgctgcgtttggtgtcaacaccaacagcgccacgcccgcagttccgcaaatagcccccaggaccgccatcaatcgtatcgggctacctagcagagcggcagagatgaacacgaccatcagcggctgcacagcgcctaccgtcgccgcgaccccgcccggcaggcggtagaccgaaataaacaacaagctccagaatagcgaaatattaagtgcgccgaggatgaagatgcgcatccaccagattcccgttggaatctgtcggacgatcatcacgagcaataaacccgccggcaacgcccgcagcagcataccggcgacccctcggcctcgctgttcgggctccacgaaaacgccggacagatgcgccttgtgagcgtccttggggccgtcctcctgtttgaagaccgacagcccaatgatctcgccgtcgatgtaggcgccgaatgccacggcatctcgcaaccgttcagcgaacgcctccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggtcgtggccgcgccaacctttgcgatccgcaagcgcgcggtcgccatcttcacgctggaacagtacgtcgaggcgggcatcatgacccgcgagcaatacgaggtcattaaaagcgccgtgattgatgatatagcggcccggctgctcctggttctcgcgcaccgaaatgggtgacttcaccccgcgctctttgatcgtggcaccgatttccgcgatgctctccggggaaaagccggggttgtcggccgtccgcggctgatgcggatcttcgtcgatcaggtccaggtccagctcgatagggccggaaccgccctgagacgccgcaggagcgtccaggaggctcgacaggtcgccgatgctatccaaccccaggccggacggctgcgccgcgcctgcggcttcctgagcggccgcagcggtgtttttcttggtggtcttggcttgagccgcagtcattgggaaatctccatcttcgtgaacacgtaatcagccagggcgcgaacctctttcgatgccttgcgcgcggccgttttcttgatcttccagaccggcacaccggatgcgagggcatcggcgatgctgctgcgcaggccaacggtggccggaatcatcatcttggggtacgcggccagcagctcggcttggtggcgcgcgtggcgcggattccgcgcatcgaccttgctgggcaccatgccaaggaattgcagcttggcgttcttctggcgcacgttcgcaatggtcgtgaccatcttcttgatgccctggatgctgtacgcctcaagctcgatgggggacagcacatagtcggccgcgaagagggcggccgccaggccgacgccaagggtcggggccgtgtcgatcaggcacacgtcgaagccttggttcgccagggccttgatgttcgccccgaacagctcgcgggcgtcgtccagcgacagccgttcggcgttcgccagtaccgggttggactcgatgagggcgaggcgcgcggcctggccgtcgccggctgcgggtgcggtttcggtccagccgccggcagggacagcgccgaacagcttgcttgcatgcaggccggtagcaaagtccttgagcgtgtaggacgcattgccctgggggtccaggtcgatcacggcaacccgcaagccgcgctcgaaaaagtcgaaggcaagatgcacaagggtcgaagtcttgccgacgccgcctttctggttggccgtgaccaaagttttcatcgtttggtttcctgttttttcttggcgtccgcttcccacttccggacgatgtacgcctgatgttccggcagaaccgccgttacccgcgcgtacccctcgggcaagttcttgtcctcgaacgcggcccacacgcgatgcaccgcttgcgacactgcgcccctggtcagtcccagcgacgttgcgaacgtcgcctgtggcttcccatcgactaagacgccccgcgctatctcgatggtctgctgccccacttccagcccctggatcgcctcctggaactggctttcggtaagccgtttcttcatggataacacccataatttgctccgcgccttggttgaacatagcggtgacagccgccagcacatgagagaagtttagctaaacatttctcgcacgtcaacacctttagccgctaaaactcgtccttggcgtaacaaaacaaaagcccggaaaccgggctttcgtctcttgccgcttatggctctgcacccggctccatcaccaacaggtcgcgcacgcgcttcactcggttgcggatcgacactgccagcccaacaaagccggttgccgccgccgccaggatcgcgccgatgatgccggccacaccggccatcgcccaccaggtcgccgccttccggttccattcctgctggtactgcttcgcaatgctggacctcggctcaccataggctgaccgctcgatggcgtatgccgcttctccccttggcgtaaaacccagcgccgcaggcggcattgccatgctgcccgccgctttcccgaccacgacgcgcgcaccaggcttgcggtccagaccttcggccacggcgagctgcgcaaggacataatcagccgccgacttggctccacgcgcctcgatcagctcttgcactcgcgcgaaatccttggcctccacggccgccatgaatcgcgcacgcggcgaaggctccgcagggccg

SEQ ID NO: 22 - DNA, 14530 bp, Artificial Sequence
  Description of Artificial Sequence Synthetic plasmid
ccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcggccgcgggctggccgaggtgctgggcaagccctacctccaggcccccatcggggtcgagagcacgaccgccttcctgcgccgcctgggcgagattctgggcctcgatccggagcccttcatcgagcgcgagaagcactcgacgctgaagcccgtgtgggatctgtggcggagtgtcacgcaggacttcttcgggacggccaatttcggaatcgtggcgaccgaaacttatgcaagaggcatccgaaactatctcgaaggcgatctcgggctgccctgcgccttcgccgtggcccgcaagaggggctcgaagaccgacaacgaagcggtgcgcggactgatccgccagcaccgtccgctcgtgctcatggggtcgatcaacgagaagatttaccttgcggaactgaaagccggtcacggcccgcaaccctctttcatcgctgcctctttcccgggtgcggcgatccggcgcgctaccggaacgcccgttatgggatatgcaggtgctacgtggttactgcaggaagtttgcaacgccctgttcgacgccctgttccacattctgcccctcgggacggagatggacagcgccgccgccacaccgacgacactgcgccgcgacttcccgtgggatgccgatgcgcaagcggccctggaccgcatcgtagaggagcatccggttctcacccggatcagcgccgcgcgtgccttgcgcgacgccgccgagaaggctgccctcgatgccggtgccgagagggtcgtgagagagactgtcgaagccctgcgtgggccgggcttcggcgagaggaagggagagaaccaatgagcgatcatgccgtcaacacgccggtccatgccgccagggcccacgggcaccgagcaccacgtgccgagttctacgtctacttcgccgtcattctgctgggcgccttcccggtggccttcgtgagctggatcgtctcgacgatccgccaccgcaggcttcccaagcgcggccccttcgcgtccgcctggttcgatgccaaggcgatcacgccgctgattttccgcgcctgaccgcaggtcaggttgcgacacgccattcgtcgtctccccaaggggcggcggattaatcgggagggcatggtgccttaccgtaacccacgccaccagcatgtggcgactaggggaggatagtaatgctcgtcaagacgcacatcacgaagattggggtcacgctgttcgccgtggctctgttctacgggttcatctacatgctttccaactcgctctttgcgacgcggccggccaccgccgttgcggtcggtgcggacgggaaagccctgactagttccatatgatagatctcaccaccaccaccaccaccactaataggccggccctccgtcgcgggcggcacccacgcccgcatcgattccaaggttcagccattgagacggctccgcttcgcgcgcaagcgcgggttgggccgactgcaagcggagagggaagcatggcactgctcagcttcgagcgaaaatatcgcgtgccggggggcacgctggtcggcggaaacctgttcgacttctgggtcggccctttctatgtcggcttcttcggggttgcgacgtttttcttcgcggccctgggtatcattctgattgcctggagtgccgtactccagggtacctggaacccccaactcatctctgtctacccgccggcccttgaatatggcctgggaggtgcacccctcgcaaaaggcgggctgtggcagatcatcacgatctgcgccactggtgccttcgtcagctgggcgctgcgcgaagtcgaaatctgccgtaagctgggcatcgggtaccacatcccgttcgccttcgcgttcgccatcctggcctacctgacgctggtgctgttccgcccggtgatgatgggcgcctggggctatgccttcccctacgggatctggacgcacctcgactgggtgtcgaacacgggctacacctacggcaacttccactacaaccctgcccacatgatcgccatctcgttcttcttcacgaacgcgctggctctggcgctgcacggcgcccttgtgctctccgcggccaaccccgagaagggcaaggaaatgcggacgccggatcacgaggatacgttcttccgcgatctggtcggctactcgatcgggacgctcggcatccaccgcctcggcctgctgctctcgctgagcgccgtcttcttcagcgccctctgcatgatcattaccggcaccatctggttcgatcagtgggtcgactggtggcaatggtgggtgaagctgccgtggtgggcgaacatcccgggaggcatcaatggctgagtatcagaacatcttctcccaggtccaggtccgcggaccggccgacctggggatgaccgaagacgtcaacctggccaaccgttcgggcgtcggtcccttctcgaccctgctcggctggttcggcaacgcccagctcggcccgatctatctcggctcgctcggcgtcctgtccctcttctcgggcctgatgtggttcttcaccatcgggatctggttctggtatcaggcgggctggaacccggccgtcttcctgcgcgacctgttcttcttctcgctcgagccgccggcacccgaatacggtctgtccttcgcggctccgctgaaggaaggcgggctgtggctgatcgcgtcgttcttcatgttcgtcgcggtctggtcctggtggggccgcacctatctccgcgctcaggcgctgggcatgggcaagcacaccgcctgggcgttcctctcggccatctggctgtggatggtgctgggcttcatccgtccgatcctcatggggtcctggtcggaagcggttccctacggcatcttctcgcacctcgactggacgaacaacttctcgctcgtccacggcaacctgttctacaaccccttccacggtctctcgatcgccttcctctacgggtcggccctgctcttcgcgatgcacggtgcgaccatcctcgcggtctcccgcttcggcggcgagcgcgagctggagcagatcgccgaccgcgggacggcagcggagcgggccgccctcttctggcgctggaccatgggtttcaacgccacgatggaaggcatccaccgctgggccatctggatggcggtcctcgtgaccctcaccggcggcatcgggatcctgctctcgggcacggtcgtggacaactggtacgtctggggccagaaccacggcatggcgccgctgaactgaggagcgatcacaatggctgacaagaccatcttcaacgatcacctcaacaccaatccgaagaccaaccttcgcctctgggtcgctttccagatgatgaagggtgcgggctgggctggcggcgtgttcttcgggacgctccttctcatcgggttcttccgggtggtcgggcggatgcttccgatccaggagaaccaggctccggcgccgaacatcaccggcgctctggagaccgggatcgagctgatcaagcatctcgtctgagacaagtctcggggcagggcggcgcgaggccgcccgctcctccaagtccgggccatatcgccggcccgggtccggggcgacaccacagcccggttcccttcctgttggcgacagggacctggtgccgtgtggaagaccgcacggcacccttttgacattcacgggaggctctgatgaccaatcccaccccgcgacccgaaaccccgcttttggatcgcgtctgctgcccggccgacatgaaggcgctgagtgacgccgaactggagcggctggccgacgaagtgcgttccgaggtcagtgataggggtagtttcttattttaggcagtttatatgaaattaagacatgcagatgtcacagtggatattgaactggtctcgaaagctcaatatcccccaaagcacaagcacaaacttcgacatcatgcagaagcgtttcccgaaccgcgtcttcgacgtgggcatcgccgagcagcatgccgtgaccttcgcggccggcctcgccggggccgggatgaagcccttctgcgcgatctattcctcgttcctgcaacggggttacgaccagatcgcccatgacgtggcgctgcagaaccttcccgtccgcttcgtgatcgaccgggcggggctcgtgggggccgatggcgcgacccatgcgggggccttcgacgttggcttcatcacttcgctgcccaacatgaccgtgatggccgcggccgacgaggccgagctcatccacatgatcgccaccgccgtggccttcgacgagggccccatcgccttccgcttcccgcggggcgagggggtgggcgtcgagatgcccgagcgcgggacggtgctggagcccggccggggccgcgtggtgcgcgaagggacggatgtcgcgatcctctccttcggcgcgcatctgcacgaggccttgcaggcggcgaaacttctcgaggccgagggggtgagcgtgaccgtggccgacgcccgcttctcgcgcccgctcgacacggggctcatcgaccagctcgtgcgccatcacgcggcgctggtaacggtggagcagggggccatgggcggcttcggcgcccatgtcatgcactatctcgccaattccggcggcttcgacgggggcctcgcgctccgggtcatgacgctgcccgaccgcttcatcgagcaggcgagccccgaggacatgtatgccgatgcggggctgcgggccgaggatatcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgcgttcaatcggaccagcggaggctggtccggaggccagacatgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccactgtcgactacgccatcatggcgacagcgcctttcctttgggttctctatatcgggcggatcgtggccggcatcaccggggcgactggggcggtagccggcgcttatattgccgatatcactgatggcgatgagcgcgcgcggcacttcggcttcatgagcgcctgtttcgggttcgggatggtcgcgggacctgtgctcggtgggctgatgggcggtttctccccccacgctccgttcttcgccgcggcagccttgaacggcctcaatttcctgacgggctgtttccttttgccggagtcgcacaaaggcgaacgccggccgttacgccgggaggctctcaacccgctcgcttcgttccggtgggcccggggcatgaccgtcgtcgccgccctgatggcggtcttcttcatcatgcaacttgtcggacaggtgccggccgcgctttgggtcattttcggcgaggatcgctttcactgggacgcgaccacgatcggcatttcgcttgccgcatttggcattctgcattcactcgcccaggcaatgatcaccggccctgtagccgcccggctcggcgaaaggcgggcactcatgctcggaatgattgccgacggcacaggctacatcctgcttgccttcgcgacacggggatggatggcgttcccgatcatggtcctgcttgcttcgggtggcatcggaatgccggcgctgcaagcaatgttgtccaggcaggtggatgaggaacgtcaggggcagctgcaaggctcactggcggcgctcaccagcctgacctcgatcgtcggacccctcctcttcacggcgatctatgcggcttctataacaacgtggaacgggtgggcatggattgcaggcgctgccctctacttgctctgcctgccggcgctgcgtcgcgggctttggagcggcgcagggcaacgagccgatcgctgatcgtggaaacgataggcctatgccatgcgggtcaaggcgacttccggcaagctatacgcgccctaggagtgcggttggaacgttggcccagccagatactcccgatcacgagcaggacgccgatgatttgaagcgcactcagcgtctgatccaagaacaaccatcctagcaacacggcggtccccgggctgagaaagcccagtaaggaaacaactgtaggttcgagtcgcgagatcccccggaaccaaaggaagtaggttaaacccgctccgatcaggccgagccacgccaggccgagaacattggttcctgtaggcatcgggattggcggatcaaacactaaagctactggaacgagcagaagtcctccggccgccagttgccaggcggtaaaggtgagcagaggcacgggaggttgccacttgcgggtcagcacggttccgaacgccatggaaaccgcccccgccaggcccgctgcgacgccgacaggatctagcgctgcgtttggtgtcaacaccaacagcgccacgcccgcagttccgcaaatagcccccaggaccgccatcaatcgtatcgggctacctagcagagcggcagagatgaacacgaccatcagcggctgcacagcgcctaccgtcgccgcgaccccgcccggcaggcggtagaccgaaataaacaacaagctccagaatagcgaaatattaagtgcgccgaggatgaagatgcgcatccaccagattcccgttggaatctgtcggacgatcatcacgagcaataaacccgccggcaacgcccgcagcagcataccggcgacccctcggcctcgctgttcgggctccacgaaaacgccggacagatgcgccttgtgagcgtccttggggccgtcctcctgtttgaagaccgacagcccaatgatctcgccgtcgatgtaggcgccgaatgccacggcatctcgcaaccgttcagcgaacgcctccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggtcgtggccgcgccaacctttgcgatccgcaagcgcgcggtcgccatcttcacgctggaacagtacgtcgaggcgggcatcatgacccgcgagcaatacgaggtcattaaaagcgccgtgattgatgatatagcggcccggctgctcctggttctcgcgcaccgaaatgggtgacttcaccccgcgctctttgatcgtggcaccgatttccgcgatgctctccggggaaaagccggggttgtcggccgtccgcggctgatgcggatcttcgtcgatcaggtccaggtccagctcgatagggccggaaccgccctgagacgccgcaggagcgtccaggaggctcgacaggtcgccgatgctatccaaccccaggccggacggctgcgccgcgcctgcggcttcctgagcggccgcagcggtgtttttcttggtggtcttggcttgagccgcagtcattgggaaatctccatcttcgtgaacacgtaatcagccagggcgcgaacctctttcgatgccttgcgcgcggccgttttcttgatcttccagaccggcacaccggatgcgagggcatcggcgatgctgctgcgcaggccaacggtggccggaatcatcatcttggggtacgcggccagcagctcggcttggtggcgcgcgtggcgcggattccgcgcatcgaccttgctgggcaccatgccaaggaattgcagcttggcgttcttctggcgcacgttcgcaatggtcgtgaccatcttcttgatgccctggatgctgtacgcctcaagctcgatgggggacagcacatagtcggccgcgaagagggcggccgccaggccgacgccaagggtcggggccgtgtcgatcaggcacacgtcgaagccttggttcgccagggccttgatgttcgccccgaacagctcgcgggcgtcgtccagcgacagccgttcggcgttcgccagtaccgggttggactcgatgagggcgaggcgcgcggcctggccgtcgccggctgcgggtgcggtttcggtccagccgccggcagggacagcgccgaacagcttgcttgcatgcaggccggtagcaaagtccttgagcgtgtaggacgcattgccctgggggtccaggtcgatcacggcaacccgcaagccgcgctcgaaaaagtcgaaggcaagatgcacaagggtcgaagtcttgccgacgccgcctttctggttggccgtgaccaaagttttcatcgtttggtttcctgttttttcttggcgtccgcttcccacttccggacgatgtacgcctgatgttccggcagaaccgccgttacccgcgcgtacccctcgggcaagttcttgtcctcgaacgcggcccacacgcgatgcaccgcttgcgacactgcgcccctggtcagtcccagcgacgttgcgaacgtcgcctgtggcttcccatcgactaagacgccccgcgctatctcgatggtctgctgccccacttccagcccctggatcgcctcctggaactggctttcggtaagccgtttcttcatggataacacccataatttgctccgcgccttggttgaacatagcggtgacagccgccagcacatgagagaagtttagctaaacatttctcgcacgtcaacacctttagccgctaaaactcgtccttggcgtaacaaaacaaaagcccggaaaccgggctttcgtctcttgccgcttatggctctgcacccggctccatcaccaacaggtcgcgcacgcgcttcactcggttgcggatcgacactgccagcccaacaaagccggttgccgccgccgccaggatcgcgccgatgatgccggccacaccggccatcgcccaccaggtcgccgccttccggttccattcctgctggtactgcttcgcaatgctggacctcggctcaccataggctgaccgctcgatggcgtatgccgcttctccccttggcgtaaaacccagcgccgcaggcggcattgccatgctgcccgccgctttcccgaccacgacgcgcgcaccaggcttgcggtccagaccttcggccacggcgagctgcgcaaggacataatcagccgccgacttggctccacgcgcctcgatcagctcttgcactcgcgcgaaatccttggcctccacggccgccatgaatcgcgcacgcggcgaaggctccgcagggccg

SEQ ID NO: 23 - DNA, 14448 bp, Artificial Sequence
  Description of Artificial Sequence Synthetic plasmid
ccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcggccgcgggctggccgaggtgctgggcaagccctacctccaggcccccatcggggtcgagagcacgaccgccttcctgcgccgcctgggcgagattctgggcctcgatccggagcccttcatcgagcgcgagaagcactcgacgctgaagcccgtgtgggatctgtggcggagtgtcacgcaggacttcttcgggacggccaatttcggaatcgtggcgaccgaaacttatgcaagaggcatccgaaactatctcgaaggcgatctcgggctgccctgcgccttcgccgtggcccgcaagaggggctcgaagaccgacaacgaagcggtgcgcggactgatccgccagcaccgtccgctcgtgctcatggggtcgatcaacgagaagatttaccttgcggaactgaaagccggtcacggcccgcaaccctctttcatcgctgcctctttcccgggtgcggcgatccggcgcgctaccggaacgcccgttatgggatatgcaggtgctacgtggttactgcaggaagtttgcaacgccctgttcgacgccctgttccacattctgcccctcgggacggagatggacagcgccgccgccacaccgacgacactgcgccgcgacttcccgtgggatgccgatgcgcaagcggccctggaccgcatcgtagaggagcatccggttctcacccggatcagcgccgcgcgtgccttgcgcgacgccgccgagaaggctgccctcgatgccggtgccgagagggtcgtgagagagactgtcgaagccctgcgtgggccgggcttcggcgagaggaagggagagaaccaatgagcgatcatgccgtcaacacgccggtccatgccgccagggcccacgggcaccgagcaccacgtgccgagttctacgtctacttcgccgtcattctgctgggcgccttcccggtggccttcgtgagctggatcgtctcgacgatccgccaccgcaggcttcccaagcgcggccccttcgcgtccgcctggttcgatgccaaggcgatcacgccgctgattttccgcgcctgaccgcaggtcaggttgcgacacgccattcgtcgtctccccaaggggcggcggattaatcgggagggcatggtgccttaccgtaacccacgccaccagcatgtggcgactaggggaggatagtaatgaagttccaagtcaaggccctcgccgccatcgccgcattcgcggcgctgccggcgctcgcgcagactagttccatatgatagatctcaccaccaccaccaccaccactaataggccggccctccgtcgcgggcggcacccacgcccgcatcgattccaaggttcagccattgagacggctccgcttcgcgcgcaagcgcgggttgggccgactgcaagcggagagggaagcatggcactgctcagcttcgagcgaaaatatcgcgtgccggggggcacgctggtcggcggaaacctgttcgacttctgggtcggccctttctatgtcggcttcttcggggttgcgacgtttttcttcgcggccctgggtatcattctgattgcctggagtgccgtactccagggtacctggaacccccaactcatctctgtctacccgccggcccttgaatatggcctgggaggtgcacccctcgcaaaaggcgggctgtggcagatcatcacgatctgcgccactggtgccttcgtcagctgggcgctgcgcgaagtcgaaatctgccgtaagctgggcatcgggtaccacatcccgttcgccttcgcgttcgccatcctggcctacctgacgctggtgctgttccgcccggtgatgatgggcgcctggggctatgccttcccctacgggatctggacgcacctcgactgggtgtcgaacacgggctacacctacggcaacttccactacaaccctgcccacatgatcgccatctcgttcttcttcacgaacgcgctggctctggcgctgcacggcgcccttgtgctctccgcggccaaccccgagaagggcaaggaaatgcggacgccggatcacgaggatacgttcttccgcgatctggtcggctactcgatcgggacgctcggcatccaccgcctcggcctgctgctctcgctgagcgccgtcttcttcagcgccctctgcatgatcattaccggcaccatctggttcgatcagtgggtcgactggtggcaatggtgggtgaagctgccgtggtgggcgaacatcccgggaggcatcaatggctgagtatcagaacatcttctcccaggtccaggtccgcggaccggccgacctggggatgaccgaagacgtcaacctggccaaccgttcgggcgtcggtcccttctcgaccctgctcggctggttcggcaacgcccagctcggcccgatctatctcggctcgctcggcgtcctgtccctcttctcgggcctgatgtggttcttcaccatcgggatctggttctggtatcaggcgggctggaacccggccgtcttcctgcgcgacctgttcttcttctcgctcgagccgccggcacccgaatacggtctgtccttcgcggctccgctgaaggaaggcgggctgtggctgatcgcgtcgttcttcatgttcgtcgcggtctggtcctggtggggccgcacctatctccgcgctcaggcgctgggcatgggcaagcacaccgcctgggcgttcctctcggccatctggctgtggatggtgctgggcttcatccgtccgatcctcatggggtcctggtcggaagcggttccctacggcatcttctcgcacctcgactggacgaacaacttctcgctcgtccacggcaacctgttctacaaccccttccacggtctctcgatcgccttcctctacgggtcggccctgctcttcgcgatgcacggtgcgaccatcctcgcggtctcccgcttcggcggcgagcgcgagctggagcagatcgccgaccgcgggacggcagcggagcgggccgccctcttctggcgctggaccatgggtttcaacgccacgatggaaggcatccaccgctgggccatctggatggcggtcctcgtgaccctcaccggcggcatcgggatcctgctctcgggcacggtcgtggacaactggtacgtctggggccagaaccacggcatggcgccgctgaactgaggagcgatcacaatggctgacaagaccatcttcaacgatcacctcaacaccaatccgaagaccaaccttcgcctctgggtcgctttccagatgatgaagggtgcgggctgggctggcggcgtgttcttcgggacgctccttctcatcgggttcttccgggtggtcgggcggatgcttccgatccaggagaaccaggctccggcgccgaacatcaccggcgctctggagaccgggatcgagctgatcaagcatctcgtctgagacaagtctcggggcagggcggcgcgaggccgcccgctcctccaagtccgggccatatcgccggcccgggtccggggcgacaccacagcccggttcccttcctgttggcgacagggacctggtgccgtgtggaagaccgcacggcacccttttgacattcacgggaggctctgatgaccaatcccaccccgcgacccgaaaccccgcttttggatcgcgtctgctgcccggccgacatgaaggcgctgagtgacgccgaactggagcggctggccgacgaagtgcgttccgaggtcagtgataggggtagtttcttattttaggcagtttatatgaaattaagacatgcagatgtcacagtggatattgaactggtctcgaaagctcaatatcccccaaagcacaagcacaaacttcgacatcatgcagaagcgtttcccgaaccgcgtcttcgacgtgggcatcgccgagcagcatgccgtgaccttcgcggccggcctcgccggggccgggatgaagcccttctgcgcgatctattcctcgttcctgcaacggggttacgaccagatcgcccatgacgtggcgctgcagaaccttcccgtccgcttcgtgatcgaccgggcggggctcgtgggggccgatggcgcgacccatgcgggggccttcgacgttggcttcatcacttcgctgcccaacatgaccgtgatggccgcggccgacgaggccgagctcatccacatgatcgccaccgccgtggccttcgacgagggccccatcgccttccgcttcccgcggggcgagggggtgggcgtcgagatgcccgagcgcgggacggtgctggagcccggccggggccgcgtggtgcgcgaagggacggatgtcgcgatcctctccttcggcgcgcatctgcacgaggccttgcaggcggcgaaacttctcgaggccgagggggtgagcgtgaccgtggccgacgcccgcttctcgcgcccgctcgacacggggctcatcgaccagctcgtgcgccatcacgcggcgctggtaacggtggagcagggggccatgggcggcttcggcgcccatgtcatgcactatctcgccaattccggcggcttcgacgggggcctcgcgctccgggtcatgacgctgcccgaccgcttcatcgagcaggcgagccccgaggacatgtatgccgatgcggggctgcgggccgaggatatcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgcgttcaatcggaccagcggaggctggtccggaggccagacatgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccactgtcgactacgccatcatggcgacagcgcctttcctttgggttctctatatcgggcggatcgtggccggcatcaccggggcgactggggcggtagccggcgcttatattgccgatatcactgatggcgatgagcgcgcgcggcacttcggcttcatgagcgcctgtttcgggttcgggatggtcgcgggacctgtgctcggtgggctgatgggcggtttctccccccacgctccgttcttcgccgcggcagccttgaacggcctcaatttcctgacgggctgtttccttttgccggagtcgcacaaaggcgaacgccggccgttacgccgggaggctctcaacccgctcgcttcgttccggtgggcccggggcatgaccgtcgtcgccgccctgatggcggtcttcttcatcatgcaacttgtcggacaggtgccggccgcgctttgggtcattttcggcgaggatcgctttcactgggacgcgaccacgatcggcatttcgcttgccgcatttggcattctgcattcactcgcccaggcaatgatcaccggccctgtagccgcccggctcggcgaaaggcgggcactcatgctcggaatgattgccgacggcacaggctacatcctgcttgccttcgcgacacggggatggatggcgttcccgatcatggtcctgcttgcttcgggtggcatcggaatgccggcgctgcaagcaatgttgtccaggcaggtggatgaggaacgtcaggggcagctgcaaggctcactggcggcgctcaccagcctgacctcgatcgtcggacccctcctcttcacggcgatctatgcggcttctataacaacgtggaacgggtgggcatggattgcaggcgctgccctctacttgctctgcctgccggcgctgcgtcgcgggctttggagcggcgcagggcaacgagccgatcgctgatcgtggaaacgataggcctatgccatgcgggtcaaggcgacttccggcaagctatacgcgccctaggagtgcggttggaacgttggcccagccagatactcccgatcacgagcaggacgccgatgatttgaagcgcactcagcgtctgatccaagaacaaccatcctagcaacacggcggtccccgggctgagaaagcccagtaaggaaacaactgtaggttcgagtcgcgagatcccccggaaccaaaggaagtaggttaaacccgctccgatcaggccgagccacgccaggccgagaacattggttcctgtaggcatcgggattggcggatcaaacactaaagctactggaacgagcagaagtcctccggccgccagttgccaggcggtaaaggtgagcagaggcacgggaggttgccacttgcgggtcagcacggttccgaacgccatggaaaccgcccccgccaggcccgctgcgacgccgacaggatctagcgctgcgtttggtgtcaacaccaacagcgccacgcccgcagttccgcaaatagcccccaggaccgccatcaatcgtatcgggctacctagcagagcggcagagatgaacacgaccatcagcggctgcacagcgcctaccgtcgccgcgaccccgcccggcaggcggtagaccgaaataaacaacaagctccagaatagcgaaatattaagtgcgccgaggatgaagatgcgcatccaccagattcccgttggaatctgtcggacgatcatcacgagcaataaacccgccggcaacgcccgcagcagcataccggcgacccctcggcctcgctgttcgggctccacgaaaacgccggacagatgcgccttgtgagcgtccttggggccgtcctcctgtttgaagaccgacagcccaatgatctcgccgtcgatgtaggcgccgaatgccacggcatctcgcaaccgttcagcgaacgcctccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcgggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggtcgtggccgcgccaacctttgcgatccgcaagcgcgcggtcgccatcttcacgctggaacagtacgtcgaggcgggcatcatgacccgcgagcaatacgaggtcattaaaagcgccgtgattgatgatatagcggcccggctgctcctggttctcgcgcaccgaaatgggtgacttcaccccgcgctctttgatcgtggcaccgatttccgcgatgctctccggggaaaagccggggttgtcggccgtccgcggctgatgcggatcttcgtcgatcaggtccaggtccagctcgatagggccggaaccgccctgagacgccgcaggagcgtccaggaggctcgacaggtcgccgatgctatccaaccccaggccggacggctgcgccgcgcctgcggcttcctgagcggccgcagcggtgtttttcttggtggtcttggcttgagccgcagtcattgggaaatctccatcttcgtgaacacgtaatcagccagggcgcgaacctctttcgatgccttgcgcgcggccgttttcttgatcttccagaccggcacaccggatgcgagggcatcggcgatgctgctgcgcaggccaacggtggccggaatcatcatcttggggtacgcggccagcagctcggcttggtggcgcgcgtggcgcggattccgcgcatcgaccttgctgggcaccatgccaaggaattgcagcttggcgttcttctggcgcacgttcgcaatggtcgtgaccatcttcttgatgccctggatgctgtacgcctcaagctcgatgggggacagcacatagtcggccgcgaagagggcggccgccaggccgacgccaagggtcggggccgtgtcgatcaggcacacgtcgaagccttggttcgccagggccttgatgttcgccccgaacagctcgcgggcgtcgtccagcgacagccgttcggcgttcgccagtaccgggttggactcgatgagggcgaggcgcgcggcctggccgtcgccggctgcgggtgcggtttcggtccagccgccggcagggacagcgccgaacagcttgcttgcatgcaggccggtagcaaagtccttgagcgtgtaggacgcattgccctgggggtccaggtcgatcacggcaacccgcaagccgcgctcgaaaaagtcgaaggcaagatgcacaagggtcgaagtcttgccgacgccgcctttctggttggccgtgaccaaagttttcatcgtttggtttcctgttttttcttggcgtccgcttcccacttccggacgatgtacgcctgatgttccggcagaaccgccgttacccgcgcgtacccctcgggcaagttcttgtcctcgaacgcggcccacacgcgatgcaccgcttgcgacactgcgcccctggtcagtcccagcgacgttgcgaacgtcgcctgtggcttcccatcgactaagacgccccgcgctatctcgatggtctgctgccccacttccagcccctggatcgcctcctggaactggctttcggtaagccgtttcttcatggataacacccataatttgctccgcgccttggttgaacatagcggtgacagccgccagcacatgagagaagtttagctaaacatttctcgcacgtcaacacctttagccgctaaaactcgtccttggcgtaacaaaacaaaagcccggaaaccgggctttcgtctcttgccgcttatggctctgcacccggctccatcaccaacaggtcgcgcacgcgcttcactcggttgcggatcgacactgccagcccaacaaagccggttgccgccgccgccaggatcgcgccgatgatgccggccacaccggccatcgcccaccaggtcgccgccttccggttccattcctgctggtactgcttcgcaatgctggacctcggctcaccataggctgaccgctcgatggcgtatgccgcttctccccttggcgtaaaacccagcgccgcaggcggcattgccatgctgcccgccgctttcccgaccacgacgcgcgcaccaggcttgcggtccagaccttcggccacggcgagctgcgcaaggacataatcagccgccgacttggctccacgcgcctcgatcagctcttgcactcgcgcgaaatccttggcctccacggccgccatgaatcgcgcacgcggcgaaggctccgcagggccg

SEQ ID NO: 24 - DNA, 48 bp, Artificial Sequence
  Description of Artificial Sequence Synthetic primer
ctgtactagtggaggatagtaatgaagcgcaaagtattgttaattccg

SEQ ID NO: 25 - DNA, 39 bp, Artificial Sequence
  Description of Artificial Sequence Synthetic primer
tgtaagatctttgtgcggcctccttactgtatttctccc

SEQ ID NO: 26 - DNA, 43 bp, Artificial Sequence
  Description of Artificial Sequence Synthetic primer
aggcaggaggaacactatgaagcgcaaagtattgttaattccg

SEQ ID NO: 27 - DNA, 48 bp, Artificial Sequence
  Description of Artificial Sequence Synthetic primer
gaccaccggacgacacttgttgtgcggcctccttactgtatttctccc

SEQ ID NO: 28 - DNA, 19 bp, Artificial Sequence
  Description of Artificial Sequence Synthetic oligonucleotide
aggcaggaggaacactatg

SEQ ID NO: 29 - DNA, 19 bp, Artificial Sequence
  Description of Artificial Sequence Synthetic oligonucleotide
caagtgtcgtccggtggtc

SEQ ID NO: 30 - DNA, 19 bp, Artificial Sequence
  Description of Artificial Sequence Synthetic oligonucleotide
catagtgttcctcctgcct

SEQ ID NO: 31 - DNA, 19 bp, Artificial Sequence
  Description of Artificial Sequence Synthetic oligonucleotide
gaccaccggacgacacttg

SEQ ID NO: 32 - DNA, 19 bp, Artificial Sequence
  Description of Artificial Sequence Synthetic oligonucleotide
aggcaggaggaacactatg

SEQ ID NO: 33 - DNA, 19 bp, Artificial Sequence
  Description of Artificial Sequence Synthetic oligonucleotide
gaccaccggacgacacttg

SEQ ID NO: 34 - protein, 9 aa, Artificial Sequence
  Description of Artificial Sequence Synthetic peptide
NSHHHHHHH

SEQ ID NO: 35 - DNA, 32 bp, Artificial Sequence
  Description of Artificial Sequence Synthetic oligonucleotide
gatctcaccaccaccaccaccaccactaatag

SEQ ID NO: 36 - DNA, 28 bp, Artificial Sequence
  Description of Artificial Sequence Synthetic oligonucleotide
ctattagtggtggtggtggtggtggtga

SEQ ID NO: 37 - DNA, 11 bp, Artificial Sequence
  Description of Artificial Sequence Synthetic oligonucleotide
ctagtcgccac

SEQ ID NO: 38 - protein, 7 aa, Artificial Sequence
  Description of Artificial Sequence Synthetic peptide
VSSGGHH

SEQ ID NO: 39 - DNA, 19 bp, Artificial Sequence
  Description of Artificial Sequence Synthetic oligonucleotide
gtgtcgtccggtggtcacc

SEQ ID NO: 40 - DNA, 18 bp, Artificial Sequence
  Description of Artificial Sequence Synthetic oligonucleotide
gtgttcctcctgcctact

SEQ ID NO: 41 - protein, 7 aa, Artificial Sequence
  Description of Artificial Sequence Synthetic peptide
HHHHHHH

SEQ ID NO: 42 - protein, 10 aa, Artificial Sequence
  Description of Artificial Sequence Synthetic peptide
HHHHHHHHHH

SEQ ID NO: 43 - protein, 13 aa, Artificial Sequence
  Description of Artificial Sequence Synthetic peptide
HHHHHHHHHHHHH

(19) United States
(12) Patent Application Publication
(10) Pub. No.: US 2007/0092943 A1
(43) Pub. Date: Apr. 26, 2007

(54) VERSATILE VECTORS FOR EXPRESSION OF FOREIGN PROTEINS IN PHOTOSYNTHETIC BACTERIA

(75) Inventors: Philip D. Laible, Villa Park, IL (US); Deborah K. Hanson, Villa Park, IL (US)

Correspondence Address: BARNES & THORNBURG LLP, P.O. BOX 2786, CHICAGO, IL, 60690-2786, US

(73) Assignee: THE UNIVERSITY OF CHICAGO, Chicago, IL (US)

(21) Appl. No.: 11/536,331

(22) Filed: Sep. 28, 2006

Related U.S. Application Data
(60) Provisional application No. 60/721,423, filed on Sep. 28, 2005.

(51) Int. Cl. C12P 21/06; C07H 21/04; C12N 15/74; C12N 1/21; C07K 14/195

(52) U.S. Cl. 435/69.1; 435/252.3; 435/471; 530/350; 536/23.7

(57) ABSTRACT

Methods for expressing and purifying foreign (heterologous) proteins in photosynthetic organisms employ expression of both heterologous membrane proteins and a means for compartmentalizing or sequestering of the protein.

This applications claims priority to U.S. Ser. No. 60/721,423 filed Sep. 28, 2005.

The United States Government has rights in this invention under Contract No. W-31-109-ENG-38 between the United States Department of Energy and the University of Chicago representing Argonne National Laboratory.

### BACKGROUND

Methods and compositions are used for expressing foreign (heterologous) genes in photosynthetic organisms and sequestering and isolating resulting heterologous proteins. Versatile vectors facilitate the cloning and expression of a wide variety of natively-folded, functionally intact target proteins in photosynthetic organisms.

“Proteins” include soluble proteins and membrane proteins. Although many systems exist for the overexpression of soluble proteins for their input into structural and functional studies and applications, some target soluble proteins prove problematic when expressed heterologously. If soluble proteins are produced too quickly or are exposed to an environment that disfavors their folding in such systems, they are often found to form large-order aggregates and precipitate, or are rapidly degraded by inherent host machinery designed to maintain order within the cell. With limited ability to change the membrane content of host cells employed routinely as expression vehicles, these soluble proteins, in most cases, are abandoned because no satisfactory system or condition can be found whereby they can be produced in useful quantity or quality. Many of these ‘problematic’ soluble proteins could benefit from an increased volume of the cytoplasmic membrane with which they can associate.

Membrane proteins are extremely important for normal cell function. They provide the means by which cells communicate, transduce signals and transport metabolites between internal compartments, and build gradients of ions which are used to fuel all ingrained activities. Membrane proteins are one of the early defenses against invading foreign organisms.

Although roughly 35% of the proteins known or expected to be found in most organisms are membrane-associated, little structural or functional information exists on these proteins relative to soluble proteins. New information on membrane protein structures would aid biologists, physicists and chemists in their understanding of important structural relationships necessary for essential protein functions in lipid bilayer environments and could provide strategies to develop drugs that need to interact with membrane functions. Quantities of native membrane-associated proteins are difficult to purify in quantities sufficient for analysis. Inasmuch as the functional properties and stability of membrane proteins are dependent upon the lipid bilayer surrounding them, these proteins often denature or otherwise deviate from their native states when removed from their natural environs. Additionally, most membrane proteins are often expressed at very low levels, in amounts insufficient for purification and crystallization. To date, the three dimensional structures of only about 60 unique membrane proteins are known, in comparison to the structures of representatives of more than 4000 families of soluble proteins.

Knowledge of the structures, and a determination of the functions, of membrane proteins would contribute greatly to understanding of biological processes and facilitate applications for clinical use. For example, structure-based rational drug design has produced powerful competitive inhibitors of cofactor binding in enzyme catalysis.

Because of their importance in cellular functions that can contribute to various disease states, membrane proteins are targets for drug discovery that impacts disease control and prevention.

Purification of membrane proteins from their host cells has been attempted by removing the proteins from hydrophobic surroundings and placing them in small detergent micelles which attempt to mimic the lipid environment. Following this solubilization process, routine chromatography or precipitation techniques (which have been perfected for soluble proteins) are utilized to purify and crystallize the solubilized membrane proteins. However, such adaptations rarely yield large amounts of the membrane protein in functional form.

Efforts have been made to create a process whereby membrane-associated proteins are over-expressed and subsequently purified from host cells of another organism (i.e., heterologous expression). To some degree, these efforts have all utilized a combination of a desired coding sequence with a foreign promoter known to induce high levels of protein synthesis. Fusion proteins, comprising a coding sequence of a desired (target) protein and the coding gene sequence of an affinity peptide, wherein the affinity peptide is attached and used to purify the desired protein product, are reported. This process provides an additional means of purifying the desired protein through chemical or enzymatic cleavage at a strategic cleavage site. No provision for maintaining the intact, tertiary and quaternary structure of the desired hydrophobic protein is reported. Purification is accomplished using for example, metal chelate affinity chromatography in nitrilotriacetic acid resins. However, no provision exists for circumventing the unique and inherent difficulties associated with purifying intact hydrophobic proteins.

Heterologous overexpression of hydrophobic proteins has been reported when coding regions of desired membrane proteins are juxtaposed with the bacterio-opsin (bop) regulatory sequences in the cell membrane of Halobacterium salinarum. However, the process does not provide for simultaneous production and sequestration or compartmentalization of the desired protein.

A heterologous overexpression system based on Hansenula polymorpha suggests the utilization of peroxisomes in which produced proteins may accumulate. However, as with the H. salinarum system, no provision exists for the simultaneous production and compartmentalization of the targeted components, inasmuch as the promoters utilized therein are for the most part constitutive.

Photosynthetic Organisms May be Hosts for Heterologous Expression of Proteins.

Members of the Rhodobacter genus are extremely robust and among the most versatile organisms known to biology. These bacteria are characterized by a metabolic diversity that allows them to adapt readily to a wide variety of environmental conditions.

They thrive in dark or well-lit environments, in the presence or absence of oxygen. They can biochemically exploit an assortment of substrates for cell growth and division, or can harvest energy from the sun for that same purpose. As an example, single members of the genus Rhodobacter are known to reduce nitrogen compounds, fix carbon dioxide, utilize carbon sources in an aerobic environment, or grow photosynthetically under anaerobic conditions—depending on resources available in their immediate vicinity. The mechanisms by which environmental cues are sensed and are used to turn on or off the biochemical machinery necessary to survive in a particular setting are complex, as is the composition of the membranes in this organism.

A heterologous overexpression system based on Rhodospirillum rubrum has been reported whereby proteins can be expressed under control of the regulatable promoters of the puh and puf operons. The photosynthetic apparatus in this organism is less evolved than Rhodobacter and lacks the puc operon encoding the structural genes of the peripheral (and highly abundant in low light regimes) light-harvesting antenna. The latter operon in Rhodobacter species is controlled by changes in both oxygen tension and light intensity, and the transmembrane proteins encoded by it are widely utilized for survival in marginal photoautotrophic conditions.

Proteins associated with the inner membranes of Rhodobacter cells (those proteins that adhere to, span, or are tethered to the membrane) are quite dynamic and are a key feature of the multifaceted nature of the organism. The robust nature of photosynthetic organisms such as Rhodobacter and their complex and dynamic membrane systems are potential cellular factories for the production of foreign proteins.

### SUMMARY

Methods and compositions for simultaneous production and sequestration of a wide variety of heterologous (foreign) proteins in photosynthetic organisms employ the design and construction of: 1) vectors carrying extended affinity tags for improved efficiency in protein purification, wherein “extended” means more than 7 residues, 2) vectors that vary in the placement of the affinity tags within the coding sequence to maintain structural and functional integrity, 3) vectors that incorporate cleavable affinity tags to yield a protein following purification that is as native as possible for structural and functional analyses; and 4) vectors that enable ligation-independent cloning (LIC) of target sequences to enable adaptation of the methods to high-throughput screening scenarios.

A method for expressing heterologous proteins in photosynthetic organisms such as the Rhodobacter species, includes the steps of producing and sequestering the protein within an inducible intracytoplasmic membrane system, wherein the protein and membrane are produced simultaneously. The protein may contain a plurality of affinity tags. The protein may be a complex of mutually co-dependent proteins. The expression of the heterologous proteins and the inducible membrane system may depend upon the same environmental stimuli.

The environmental stimuli activate a puf promoter or a puc promoter from the Rhodobacter genus. The coding sequence for the heterologous protein is inserted within the puf operon or the puc operon of the Rhodobacter genus. The inducible membrane system is controlled by the same environmental stimuli which induce expression of genes controlled by the puf promoter or the puc promoter of the Rhodobacter genus, including the target gene of interest. Examples of environmental stimuli include oxygen tension and light.

A method for producing and sequestering a functional protein within the Rhodobacter intracytoplasmic membrane wherein the expression of the membrane protein is under control of a Rhodobacter inducible promoter and wherein the functional protein is synthesized at the same time the sequestering membrane is synthesized includes the steps of:

a) supplying a DNA sequence containing the code for the target functional protein under control of a Rhodobacter inducible promoter and a host strain that produces inducible intracytoplasmic membranes in response to the same environmental cues; and

b) subjecting the resulting plasmid-bearing Rhodobacter strain to the environmental cue.

In the expression systems disclosed herein, autoinduction of both protein synthesis and intracytoplasmic membrane synthesis occurs as, for example, the oxygen tension of the culture decreases as the cell density increases.

The Rhodobacter Expression System offers several additional advantages over E. coli-based alternatives. The experimenter has more control over rates of protein expression in Rhodobacter than in E. coli. Slower, but nonetheless complete, induction in Rhodobacter is possible since this process is automatic and is controlled by oxygen tension. The resulting kinetics of induction and semi-aerobic growth rate are correspondingly slower in Rhodobacter and may shift the equilibrium towards the production of the folded, functional state of the target protein. Furthermore, induction in the Rhodobacter system is accompanied by the concomitant synthesis of new intracellular membranes which are available to newly synthesized proteins. If the target protein requires membrane association for its integrity, Rhodobacter possesses a greatly increased membrane surface area with which it can interact.

A method for purifying transmembrane proteins appends an affinity tag to the protein. An advantage is that the tag facilitates simple, rapid, and less disruptive extraction of the formed protein from its native membrane environment so that the protein retains its structural and functional integrity.

DNA sequences that transcribe mRNA, include a puf-promoted or a puc-promoted gene that results in a stable transcript and the translation of biologically active polypeptides linked to an affinity peptide that will also result in the simultaneous isolation/purification of the polypeptides in their native state.

Generally, light induced growth is facilitated in photosynthetic bacteria through the absorption of photons by specialized light-harvesting (LH) complexes, known as antennae. These antennae transfer excited states to reaction centers (RC) where primary charge separation occurs.

### BRIEF DESCRIPTION OF THE DRAWINGS

FIG. 1: Diagram of the broad-host-range expression vector used for ligation-dependent cloning with 7-member, C-terminal histidine tag. The puf operon of the Rhodobacter species of photosynthetic bacteria encodes six transmembrane proteins of the photosynthetic apparatus, that has been cloned into a broad-host-range vector (based upon pRK404). To facilitate the expression of foreign genes, the highest expressed native genes (pufB and pufA) have been replaced by a multiple cloning site (MCS). A region of stable hairpin structures is located between the MCS and the pufL gene; the major oxygen-regulated puf promoter is indicated (P). Foreign genes are mobilized into this vector by standard restriction endonuclease and ligation strategies after proper amplification. Foreign genes are fused in frame to a vector-based, seven-membered, C-terminal histidine tag (7HT) followed by appropriate translation terminators (stop codons; *). Restriction sites in bold are unique in this expression vector.

FIG. 2: Diagram of the broad-host-range expression vector used for ligation-dependent cloning with 10-member, C-terminal histidine tag.

FIG. 3: Diagram of the broad-host-range expression vector used for ligation-dependent cloning with 13-member, C-terminal histidine tag.

FIG. 4: Diagram of the broad-host-range expression vector used for ligation-dependent cloning with 7-member, N-terminal histidine tag.

FIG. 5: Diagram of the broad-host-range expression vector used for ligation-dependent cloning with 10-member, N-terminal histidine tag.

FIG. 6: Diagram of the broad-host-range expression vector used for ligation-dependent cloning with 13-member, N-terminal histidine tag.

FIG. 7: Diagram of the broad-host-range expression vector used for ligation-dependent cloning with 7-member, N-terminal histidine tag followed by a protease site for cleavage by such from Tobacco Etch virus (TEV).

FIG. 8: Diagram of the broad-host-range expression vector used for ligation-independent cloning (LIC) with 7-member, C-terminal histidine tag.

FIG. 9: Diagram of the broad-host-range expression vector used for ligation-independent cloning with 10-member, C-terminal histidine tag.

FIG. 10: Diagram of the broad-host-range expression vector used for ligation-independent cloning with 13-member, C-terminal histidine tag.

FIG. 11: Diagram of the broad-host-range expression vector used for ligation-independent cloning with 7-member, N-terminal histidine tag.

FIG. 12: Diagram of the broad-host-range expression vector used for ligation-independent cloning with 7-member, N-terminal histidine tag followed by a protease site for cleavage by such from Tobacco Etch virus.

FIG. 13: Diagram of the broad-host-range expression vector used for ligation-dependent cloning with an N-terminal membrane anchor/linker domain and a 7-member, C-terminal histidine tag.

FIG. 14: Diagram of the broad-host-range expression vector used for ligation-dependent cloning with an N-terminal, cleavable signal sequence and a t-member, C-terminal histidine tag.

FIG. 15: Small volume (80 mL) cultures of expression strains of Rhodobacter are grown semi-aerobically. Coordinated synthesis of target protein and membrane is autoinduced as oxygen tension lowers when the cell density increases.

FIG. 16: Screening for successful Rhodobacter expression and ICM insertion using 80-mL cell cultures grown semi-aerobically. Whole cell lysates (bottom) and membrane fractions (top) are extracted and analyzed using Western blotting techniques (anti-His; Novagen). Overexpressed bands are not always clearly visible in Coomassie-stained gels. Westerns are conclusive, general, and help identify membrane proteins with anomalous mobility on gels.

FIG. 17: Quantitation of heterologous expression of membrane in Rhodobacter. Western blots (anti-His antibody; Novagen) with well-characterized controls are employed to probe the level of expression of membrane proteins from E. coli (APC#s) in Rhodobacter ICMs. Experimental membranes from expression strains are compared with membranes carrying his-tagged reaction centers (expressed at 1 mg/L culture; +control) and with membranes from a recombinant strain lacking a cloned gene (−control). Any target protein expressed at or above the +control level is considered a “hit” (the two * in the above gel and blot). The expression levels of some targets rival those of native ICM proteins that can be purified to yields of >10 mg/L culture.

FIG. 18: Determination of the cellular localization in Rhodobacter of heterologously expressed membrane proteins is simplified by the presence of the polyhistidine tag. In this analysis, data are presented on an equal volume basis rather than on an equal protein basis. Here, target protein (APC 951) is found almost exclusively in the membranes. Target protein that is found in the soluble fraction results from small membrane fragments that do not pellet during ultracentrifugation; co-purifying host proteins reside quantitatively in the soluble fraction.

FIG. 19: Determination of the cellular localization in Rhodobacter of membrane proteins heterologously expressed from pRKLICHT1Dpuf. No differences in expression levels are apparent when comparing results from the same gene expressed from pRKPLHT1Dpuf or pRKLICHT1Dpuf.

FIG. 20: Determination of the level of Rhodobacter production of a soluble protein, ILR1, derived from Arabidopsis thaliana. Analysis from Western blots of whole cell lysates, where signals from the target protein can be compared to signals from well-characterized controls (porin, a β-barrel membrane protein expressed at >10 mg/L cell culture, and reaction centers produced by an engineered strain where expression has been downregulated to 1 mg L cell culture), suggests that ILR1 is produced in Rhodobacter cells at a level that equals or exceeds 2 mg/L of cell culture.

FIG. 21: A typical set of oligonucleotides used to amplify and subsequently clone a target membrane protein gene (APCO0809) into pRKLICHT1Dpuf. Boxes depict the LIC overhangs generated by T4 DNA polymerase digestion; underlined bases denote the ribosome binding site; long dashed lines represent the regions that are complementary to the template (target gene); and in circle are non-complementary, obligate bases that are necessary for generation of the LIC overhang.

FIG. 22: A typical set of oligonucleotides used to amplify and subsequently clone a target membrane protein gene (APC00809) into pRKPLHT1Dpuf. Boxes depict “dummy” bases that enable efficient digestion of the amplicon by the restriction enzymes;

circle denotes the SpeI site, black and underlined is the consensus Rhodobacter ribosome binding site (RBS); arrow is the six base spacer between RBS and initiation codon; long dashed line is the region complementary to the template (target gene); and dotted line denotes the BglII site.

FIG. 23: A typical set of oligonucleotides used to amplify and subsequently clone a target membrane protein gene (APC00809) into pRKLICHT1Dpuf. Box depicts the LIC overhang that is generated by T4 DNA polymerase digestion; underlined bases denote the ribosome binding site; long dashed line represents the region that is complementary to the template (target gene); and in circle are non-complementary, obligate bases that are necessary for generation of the LIC overhang.

FIG. 24: LIC handles for amplified target genes compatible for insertion into pRKLICHT1Dpuf are generated by 3′-5′ exonuclease activity of T4 DNA polymerase in the presence of excess dATP. The resulting Tₘs of the overhangs are sufficient to allow the transformation of competent E. coli to tetracycline resistance after a brief annealing process at room temperature.

FIG. 25: Strategy employed to clone target membrane protein genes into pRKPLHT1Dpuf using restriction enzymes SpeI and BglII. This vector is designed to fuse a C-terminal, seven-membered histidine tag. The protein sequence of the tag and “linker” amino acids are shown in single letter code.

FIG. 27: LIC strategy employed to clone target membrane protein genes into pRKLICHT1Dpuf using semi-automated methodologies. This vector is designed to fuse a C-terminal, seven-membered histidine tag (such is partially displayed). “Linker” residues between target gene and tag are shown.

### DETAILED DESCRIPTION

Expression of heterologous (foreign) proteins is achieved the introduction of gene sequences encoding the proteins into photosynthetic organism e.g. bacteria of the genus Rhodobacter.

A suite of expression vectors makes the Rhodobacter membrane protein expression system a versatile tool for functional and structural studies and possibly large-scale structural and functional genomics efforts. These vectors limit interference of affinity tags in the native folding of the target protein, thus helping it retain its native structure and function and increasing expression yields. If the tag's placement and composition cannot achieve this goal, then other vectors include sites enabling removal of affinity tags following purification of the protein of interest. Other vectors are engineered to facilitate cloning of the target gene in a manner that is not dependent upon restriction endonuclease digestion, enabling the cloning of genes that would otherwise be excluded because they contain sites for the cloning enzymes within their coding sequences.

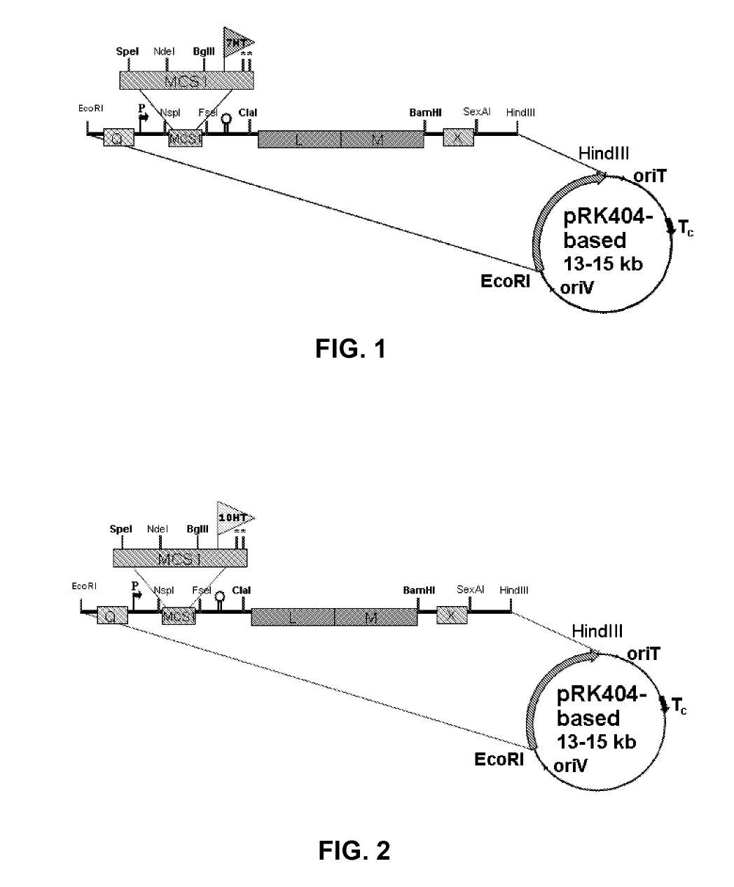

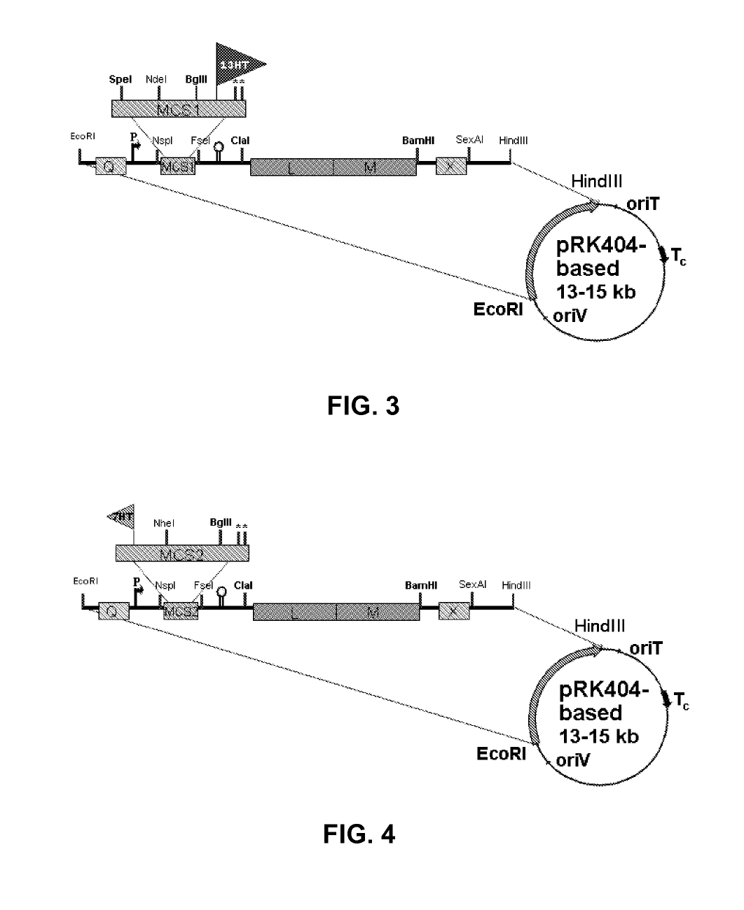

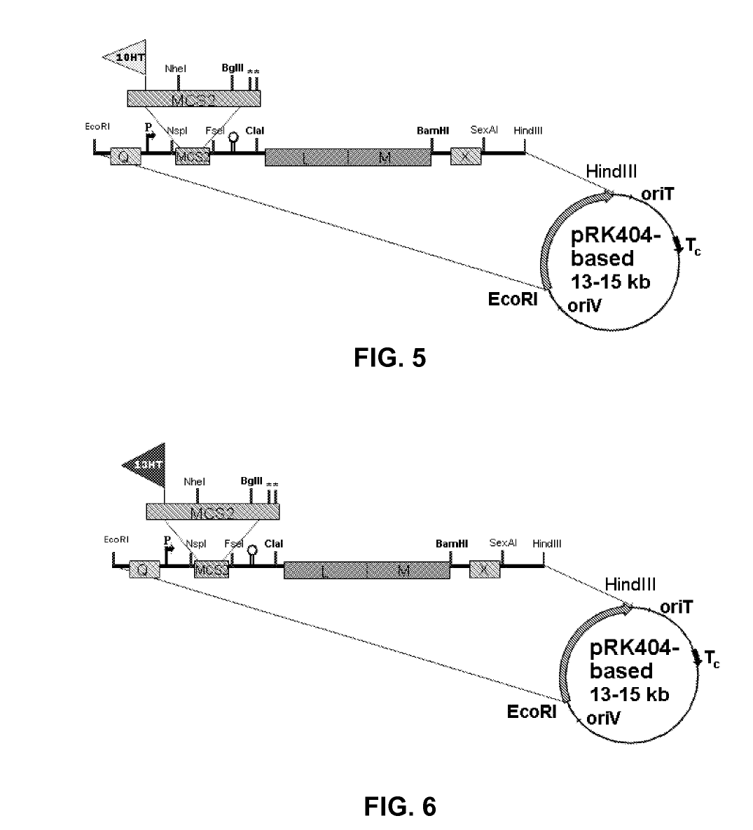

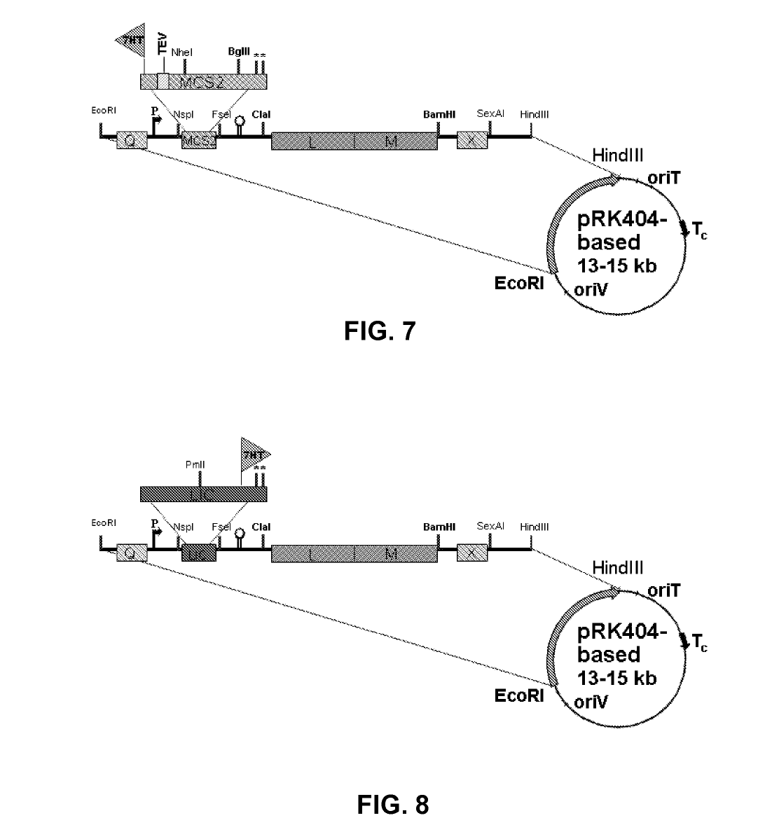

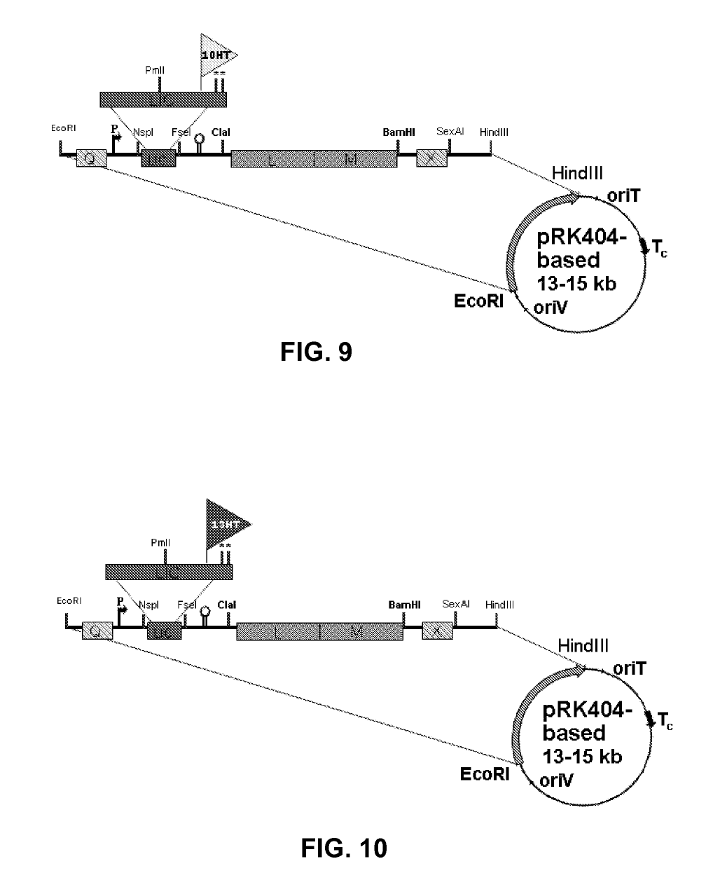

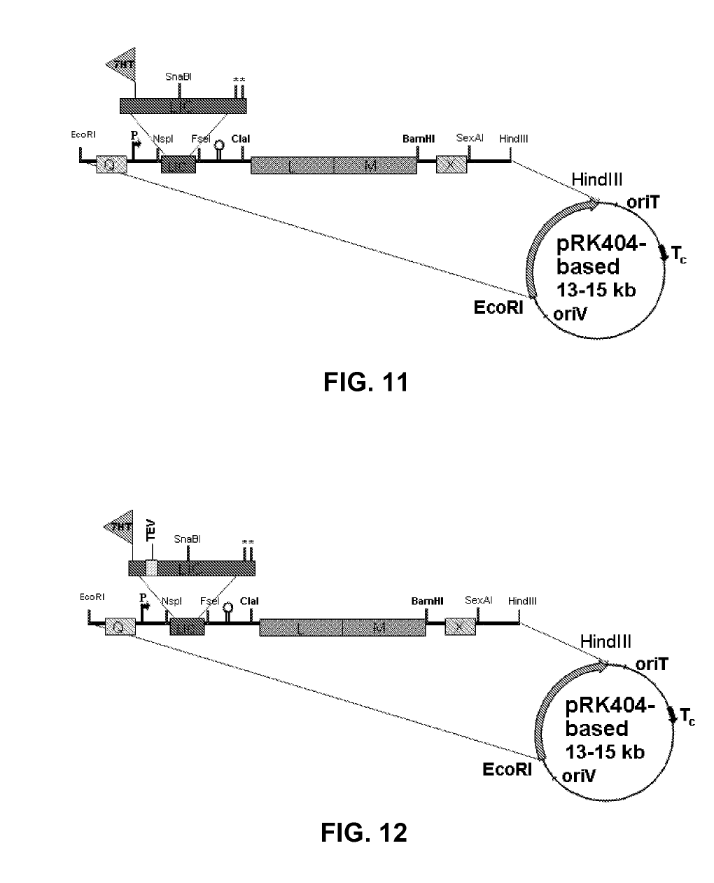

The broad-host-range expression vectors for Rhodobacter disclosed herein, include vectors with extended tags engineered to be positioned at the C-terminal or at the N-terminal end of a protein of interest (a target protein). Some of these extended tags also include a cleavable peptide moiety that is recognized by a peptide cleavage enzyme to separate the tag from the protein of interest during purification. Some of these vectors have cloning sites that enable ligation independent cloning (LIC) of a nucleic acid sequence encoding a protein of interest into the vector. Others append native-ICM-protein-derived membrane anchors and signal sequences that help to target the membrane to a particular cellular compartment to increase levels of expression of target proteins in functional form. These broad-host-range expression vectors disclosed herein include one of the features listed below (illustrated in FIGS. 1-14 and sequences disclosed herein):
(i) N-terminal 7×His tag for ligation independent cloning
(ii) N-terminal 7×His tag followed by cleavage site for Tobacco Etch Virus protease for ligation-independent cloning
(iii) N-terminal 7×His tag
(iv) N-terminal 7×His tag followed by cleavage site for Tobacco Etch Virus protease
(v) C-terminal 10×His tag for ligation-independent cloning
(vi) C-terminal 13×His tag for ligation-independent cloning (14386 bp)
(vii) C-terminal 7×His tag for ligation-independent cloning
(viii) C-terminal 10×His tag
(ix) C-terminal 13×His tag.
(x) Broad-host-range expression vector used for ligation-dependent cloning with an N-terminal membrane anchor/linker domain and a 7-member, C-terminal histidine tag.
(xi) Broad-host-range expression vector used for ligation-dependent cloning with an N-terminal, cleavable signal sequence and a t-member, C-terminal histidine tag.

The N-terminal or C-terminal tag can include any affinity tag that is of suitable length to promote better access to a purification system, such as for example, an immobilized metal ion affinity chromatography (IMAC). The N-terminal or C-terminal tag can also include a spacer or a linker that provides extended length for an affinity tag. For example, an affinity tag can include a spacer or a linker and a stretch of 6 or 7 histidine residues (spacer/linker plus his-tag). The spacer or a linker generally includes a stretch of random or non-random amino acids. The spacer or linker can range from about 1 to about 50 amino acids; 1 to about 20; 1 to about 10; or 1 to about 5 amino acids in length. The spacer or linker may or may not exhibit affinity for a purification system. The spacer or linker in combination with an affinity tag can range in length from about 5 amino acids to about 50 amino acids; from about 10 amino acids to about 20 amino acids; or from about 15 amino acids to about 30 amino acids in length. An affinity tag may also have a longer stretch of affinity residues.

Broad-host range vectors are capable of replicating in more than one host species. For example, vectors disclosed herein are capable of replicating in E. coli, Rhodobacter and other host species.

Host strain Rhodobacter sphaeroides ΔΔ11 (DeltaDelta11) was deposited in the ATCC and the accession number is designated as PTA-5921. This host strain has an increased capacity for incorporating heterologously expressed membrane proteins into its intracytoplasmic membranes. This engineered host lacks three native transmembrane complexes of the photosynthetic apparatus that normally populate the intracytoplasmic membrane in the wild-type organism.

A protocol was developed to facilitate the parallel induction of foreign proteins and host membranes. A heterologous protein is created and encapsulated in its natural state. The protein can be a membrane protein, a membrane-anchored protein, a soluble protein, a protein targeted to a specific cellular compartment, one protein, separate proteins, or a complex of mutually co-dependent proteins, such as a multi-subunit membrane-associated protein complex.

Different promoters are suitable, which respond to the same environmental stimuli, by actuating target promoters, to simultaneously induce foreign protein formation and sequestration. The co-expressed intracytoplasmic membrane (ICM) serves as a means to simultaneously compartmentalize, and therefore segregate, the developing heterologous membrane-bound proteins from the majority of other cellular components. The system has also produced functional, soluble proteins from genes derived from an unrelated organism.

Broad-host-range plasmids/vectors have been engineered to facilitate the cloning, expression and purification process. Generally, the fragment of host-chromosomal DNA containing the operon for producing the LH/RC machinery is transferred to a vector. The gene for the desired protein is then inserted to replace one or more genes of the operon. When this expression plasmid is transferred back to the photosynthetic host organism, the target protein is generated when the culture is subjected to the environmental cues that are specific for activating the promoter of that particular operon.

In Rhodobacter species, cells become pigmented as the ICM develops. This new membrane takes the form of vesicles. ICM is contiguous with a cell membrane. The interior of these vesicles contains periplasmic components. For example, a region of the ICM houses the reaction center (RC), which in photosynthetic organisms comprises a central complex of pigments and proteins. The RC is comprised of three separate components, or subunits, called H (heavy), M (medium) and L (light) based on the way these units migrate in an electric field. RC complexes house the cofactors of the photosynthesis complex, which include bacteriochlorophylls, bacteriopheophytins, quinones and a non-heme iron.

Upon cell disruption, the vesicles break apart from the cell membrane, thus becoming sealed “inside-out” particles, termed chromatophores. These vesicles (basically ICM) are easily isolated by virtue of their size. Chromatophores are much smaller than cellular debris and thus remain soluble during low-speed centrifugation. Then, during brief ultracentrifugation, they are readily separable from cellular components in forming a pellet. This pellet is rich in ICM. Therefore, proteins residing in the ICM are already significantly purified following these two simple fractionation steps with a total duration of typically less than two hours, and often less than one hour. Target proteins which are truly soluble will be found in either the cytoplasm or the periplasm. The supernatant from this brief ultracentrifugation contains both of these cellular compartments and would be used as starting material for the purification of the majority of the ‘problematic’ soluble proteins expressed in this system.

Rhodobacter produces large quantities of membrane that is filled with proteins of the photosynthetic apparatus. Using methods and compositions described herein, the photosynthetic proteins are replaced with foreign proteins. The Rhodobacter genus of photosynthetic bacteria can produce large quantities of intracytoplasmic membrane;

placing the expression of heterologous proteins under control of a promoter that controls synthesis of intracytoplasmic membrane components induces expression of the heterologous protein as well. Among the bacteria in the Rhodobacter genus, R. sphaeroides and R. capsulatus are suitable for use in the protein production and isolation method disclosed herein.

The Rhodobacter genus of photosynthetic bacteria can be grown in a variety of conditions, such as anaerobic, semi-aerobic, aerobic, light or dark. This is because the cytoplasmic membrane in Rhodobacter contains components of the respiratory chain, transport systems, and other energy-transducing complexes. The physiology of this genus under each of these conditions is different.

For example, when Rhodobacter cultures are switched from aerobic chemotrophic conditions to phototrophic growth conditions, large quantities of a new intracytoplasmic membrane (ICM) that houses the newly synthesized photosynthetic machinery are induced. This ICM is formed as invaginations of the cytoplasmic membrane and in its nascency, is contiguous with the cytoplasmic membrane. Since it houses the newly synthesized photosynthetic machinery of the cell, the lipid, chemical, and protein composition, and its kinetics of biogenesis differ from the cytoplasmic membrane.

Rhodobacter can also be induced to synthesize ICM in dark-grown cultures which are limited for oxygen, since this stimulus also directs the organism to prepare for a switch from oxidative phosphorylation to anaerobic phototrophic growth.

Rhodobacter is induced to synthesize ICM and ICM-protein, either native or foreign. During cell disruption, the ICMs break away from the cytoplasmic membrane to become discrete entities with physical properties that are different from other cellular components. Inasmuch as the cells become pigmented as these ICMs form, this phenomenon was exploited to indicate the presence of heterologous proteins formed concomitantly with the ICMs. Therefore, the heterologous proteins residing in the ICMs are easily isolated from other protein-containing cellular fractions.

To facilitate heterologous protein purification (through isolation of the heterologous protein from other ICM components), an affinity tag is engineered into the protein-coding sequence. The affinity tag is used to readily sequester the heterologous proteins in native form by chromatography with the correspondingly compatible resin. This results in a 4-5 hour purification protocol, versus the more than three day isolation procedure provided by the state-of-the-art for the purification of unengineered proteins from native hosts.

Intracytoplasmic Membrane and Rhodobacter Operon Details

The intracytoplasmic membrane (ICM) is formed when photosynthetic bacteria are switched from chemotrophic conditions to phototrophic growth conditions or when grown in the absence of light and limited oxygen.

The ICM forms from invaginations of the cell membrane and is thus contiguous with the cell membrane, while also having different characteristics vis-a-vis the cell membrane. The ICM differs from the cell membrane in its kinetics of biogenesis. Specifically, the ICM forms when ICM-protein is being actively expressed and folded, an event which occurs separate from the formation of the cell membrane.

The majority of natural ICM protein belongs to three transmembrane protein complexes of the photosynthetic apparatus: the reaction center (RC) and the two different light harvesting complexes (LH1 and LH2). The puf operon, and specifically the puf promoter, coordinates expression of Light Harvesting Complex 1 (LH1) and RC complexes. The puc operon, coordinates expression of the Light Harvesting Complex 2 (LH2), via its puc promoter. The puf operon will be discussed first.

The puf operon encodes six transmembrane proteins, specifically the two subunits of the LH1 complex, (the genes for the subunits represented as A and B in the drawing, respectively), the L and M subunits of the RC complex, and two regulatory proteins, PufQ and PufX, which are present in small amounts in the membrane. A region of stable hairpin structures is located between the pufA and pufL genes. While the puc promoter for the LH2 complex is controlled by both light and oxygen, the puf promoter, located upstream of pufQ, directs synthesis of RC and LH1 complex and is controlled solely by oxygen tension. At high oxygen tensions, the puf operon is repressed. When the oxygen tension is lowered, transcription of the puf operon is induced, and the transmembrane proteins that it encodes are produced in relative stoichiometries, determined in part by mRNA stability. The hairpin structure located between pufA and pufL confers this stability to varying degrees by protecting the transcript from exonuclease digestion, according to the positions of puf genes relative to its own location. The hairpin structures serve as a means for blocking exonuclease action beyond the location of the hairpin. The result of this blocking mechanism is an increase in mRNA stability leading ultimately to production of a larger quantity of the protein of interest. The LH1-B and LH1-A proteins are present in 15-20 fold excess over the RC-L and RC-M subunits because the stable hairpin structure prevents degradation of the mRNA of the former.

All of the puf operon proteins are inserted into the developing ICM, whose synthesis is induced coordinately.

Transcription of the operon and synthesis of the ICM is induced in the lab by growing cells under semi-aerobic, chemoheterotrophic conditions in the dark per the protocols provided herein. Under these conditions, complexes of the photosynthetic apparatus are synthesized and assembled and the ICM is produced even though the cell is not using these components to grow.

The R. sphaeroides operon is cloned into a modified version of broad-host-range vector pRK404, an 11.2 kb derivative of pRK292 which carries the polylinker from pUC9 and tetracycline resistance. It is transferred to Rhodobacter via conjugation with E. coli donor strain S17-1; its copy number in Rhodobacter strains is 4-6/cell. Plasmid pRK404 was subsequently engineered to remove a second EcoRI site, and the HindIII site in the polylinker has also been removed to leave a single HindIII site within the puf operon. This modified vector is designated pRK442(H). These modifications facilitated the shuttling of singly- or multiply-mutated L and M genes in and out of the plasmid. For expression of mutant or wild-type RCs, plasmid pRKHTpuf (or a derivative of it) is used to complement, in trans, a strain of R. sphaeroides (ΔΔ11) that carries an engineered deletion of the chromosomal copy of this operon. The genes for the LH2 complex are also deleted in strain ΔΔ11, thus the phenotype of this strain is LH1^{s} LH2^{s}RC^{s}.

Site-specific mutagenesis is used to append a seven-histidine tail to the C-terminus of the M subunit of RCs of R. capsulatus. This tail is on the periplasmic surface of the pigment-protein complex and associates with Ni- or Co-NTA (nitrilotriacetic acid) resin for rapid IMAC. Starting from a cell suspension, extremely pure RCs are isolated using a 4-5 hour protocol. The previous purification methodology took 3 days and produced complexes that were less pure.

Inasmuch as the His-tag improved the R. capsulatus RC purification so dramatically, it was added to a vector for production of R. sphaeroides RCs. This modification is useful for R. sphaeroides RCs because, unlike R. capsulatus RCs, the former have a greater propensity to form diffraction-quality crystals. To facilitate the addition of an analogous His-tag to this RC, an expression vector carrying a his-tagged R. sphaeroides M gene was obtained. In a multi-step cloning strategy, the His-tagged M gene was added to the R. sphaeroides system for site-directed mutagenesis.

By coupling this expression system with the IMAC purification protocol discussed herein, large quantities of exceptionally pure RCs from both mutant and wild-type strains of R. sphaeroides are obtained.

Based on the success of the system for expressing native and mutant RCs, the expression vectors were modified to facilitate the heterologous expression of any target gene in Rhodobacter. These engineered vectors are designed to place expression of a foreign gene under control of the oxygen-regulated puf operon promoter (P). The position of the gene relative to the region of stable hairpin structure in the operon dictates the relative level of expression. A multiple cloning site (MCS) replaces genes of the LH1 complex for high-level expression of the foreign protein. A multiple cloning site that allows for insertion of the foreign gene in place of reaction center genes (L and M) to obtain a moderate expression level. Dual expression of two genes is possible by combining these strategies.

Other broad-host-range vectors, host-specific vectors, or vectors utilizing ligation-independent cloning (LIC) strategies are also appropriate vehicles to facilitate protein expression in trans. LIC protocols utilize the proof-reading capabilities inherent in some DNA polymerases to generate lengthy complementary cohesive ends between the insert and vector which when annealed in the absence of ligating enzymes yield molecules that transform organisms with high efficiency. Vectors containing an N-terminal membrane anchor/linker domain help to target a fused heterologously-expressed protein to the ICM.

In a similar manner, vectors have been designed to fuse an N-terminal, cleavable signal sequence to the coding sequence of the target protein in order to direct a soluble protein or the N-terminus of a membrane protein to the periplasmic space. Conjugation is utilized to shuttle LIC plasmids into Rhodobacter.

For extended tags, because many target proteins have low affinity for Ni-NTA resin with just a simple heptaoligomeric histidine tag fused to the end of its normal amino acid chain, in the absence of a linker, more residues were successful in improving adherence of target proteins to resin, allowing more quantitative removal of impurities that bind either non-specifically or with lower affinity to these columns; N-terminal tags solved the problem that some targets have a buried C-terminus that is inaccessible to chromatographic resin; extended cleavable tags addressed the problem that most structural biologists prefer to work with native protein in crystallization trials over ‘inferior’ products with tags still attached for crystallization trials; and LIC strategies eliminate concerns about sites for cloning enzymes within the sequence of the gene of interest and increase the speed by which expression constructs are generated.

Cloning with vectors featuring extended tags, N-terminal tags, extended cleavable tags, and designs enabling LIC strategies was achieved. Vectors employing N-terminal tag strategies work well and are a ‘salvage’ method employed if C-terminal tags are not satisfactory (e.g., expression yields are low, protein cannot be purified because tag is inaccessible, or tag prevents proper folding of the protein and thus disrupts its function);

cleavable tags are useful although the protease employed is not compatible with many commonly used surfactants; and the LIC strategy is such that for example, 96 expression clones are created in one experiment—a paradigm shift for previous results using Rhodobacter at a pace of one gene at a time.

For purification processes, associated with isolating the generated proteins, a suitable moiety with an affinity for a predetermined structure is appended to the generated protein for subsequent separation. The His-tag improves the ability to purify and manipulate RCs for functional studies. A polyhistidine tail (HT) is inserted in frame at the C-terminus of the MCS before stop codons (*) which terminate protein translation. This HT expedites purification of the expressed protein. The histidine tag also can be attached to the N-terminus. Other tags also are appropriate, including, but not limited to intein, maltose binding protein, and small peptide tags with high-affinity antibody-based recovery systems. A myriad of suitable peptide tags is commercially available, including, but not limited to, E-tag™ of GE Healthcare, Inc., Piscataway, N.J., and the S-tag™ of Novagen, Inc., Madison, Wis. Any of the attached tags can be designed to be cleaved with a compatible protease.

The His-tag facilitates the use of different surfactants with a wider range of properties to remove the complex from its native membrane environment. For example, when IMAC protocols are used in combination with a mild charged detergent (which is incompatible with traditional ion exchange chromatography), the cofactors of the resulting product remain in their native states as evidenced by spectral properties—dimeric bacteriochlorophyll in R. capsulatus RCs absorbs at its native 870 nanometer position versus a shift to 850 nm when other detergents are utilized. Small crystals of His-tagged RCs of R. capsulatus were obtained.

IMAC was also used to isolate LH1RC superassemblies in large quantity for crystallization trials. The non-covalent association between the RC and LH1 is strong enough to allow purification of the entire superassembly utilizing the single poly-histidine tail on the RC. Crystals of the superassembly were obtained.

The His tag also enables the changing of surfactants after removal of the complex from the native lipid bilayer. The functional or structural integrity of the complex is maintained during the purification process. In fact, four different types of spectroscopic experiments that measure electron transfer, proton transfer, or energy transfer reactions in the RC have indicated that the poly-histidine tag does not interfere with the normal functions of the complex.

Methods for Purifying the Multi-subunit RC and the LH1/RC

Superassembly complexes with a single his-tag were adapted to exploit the Rhodobacter heterologous expression system to co-purify proteins which are members of larger membrane complexes. This adaptation requires and enables the simultaneous expression of interacting proteins. Genes for many proteins that associate into functional complexes are organized into conserved DNA segments. The ability to express clusters of mutually dependent proteins enables methods in which systematic co-expression of two or more membrane-associated proteins results in successful production of proteins and/or complexes heretofore recalcitrant to efforts of mono-molecular expression.

Coordinated expression of multiple genes is accomplished by shuttling a gene cluster, containing one gene that is affinity tagged (such as with histidine), into one of the above Rhodobacter expression plasmids. If the members of the cluster physically interact, the single protein which is affinity tagged will facilitate purification of the entire complex, thereby allowing for the identity of proteins which associate to form a functional multi-subunit macromolecular membrane-associated machine. A vector that was designed for the tandem expression of two genes whose protein products associate in a stoichiometry other than 1:1.

Construction of Versatile Vectors

An expression vector the puf operon (FIG. 1) was cloned into pRK442, a modified version of the broad-host-range vector pRK404, an 11.2 kb derivative of pRK292 which carries the polylinker from pUC9 and tetracycline resistance. Later, a more generalized ‘platform’ version was engineered that allowed for introduction of foreign genes in place of structural genes of the photosynthetic apparatus. The best yield of heterologous expression was through extensive testing with several foreign genes obtained with a vector that placed a multiple-cloning-site (harboring recognition sequences for SpeI, NdeI, and BglII) in place of the pufB and pufA genes. Synthesis of the foreign protein is directed by the oxygen-/light sensitive puf promoter. Routinely, the foreign genes are amplified such that a SpeI site is inserted at the N-terminus and a BglII site is appended to the C-terminus. Cloning of the amplicon using these (or compatible) sites inserts the gene into the vector such that it is fused in frame to a C-terminal 7×His tag followed by two stop codons.

The platform vectors are based upon a large (11.2 kb) broad-host-range vector, pRK404, whose sequence was largely unknown. For ease in designing future constructs, the sequence of the pRK404 derivative being used in the project was determined [2], with assistance from MWG Biotech (Highpoint, N.C.). Knowledge of the vector sequence has been of extreme utility in design and construction of the later generations of expression vectors described herein. Because this vector is large, smaller, broad-host-range vectors (derivatives of pBBR1 were evaluated; [3-6]) that carry a variety of antibiotic resistance genes and extensive multiple cloning sites. Although higher copy number was expected, surprisingly, expression from these vectors was lower than those for genes borne on pRK404-based plasmids.

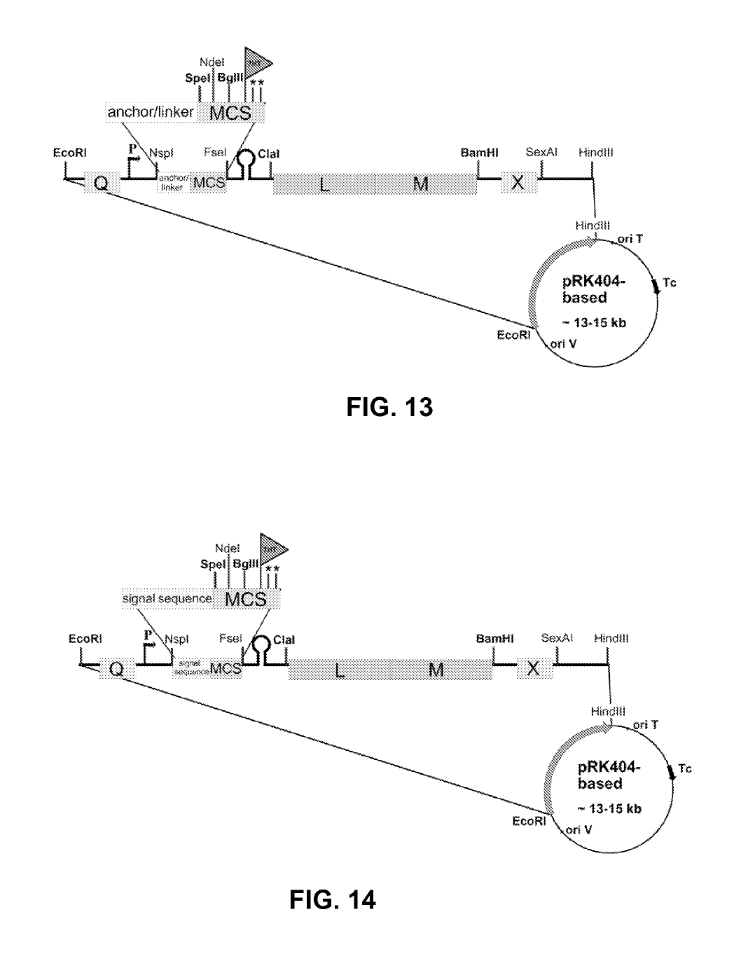

Genes for some target proteins may fail to encode compartmentalization signals that are recognized by the Rhodobacter host. Thus, a platform vector was constructed that encodes an N-terminal membrane anchor/linker domain derived from cytochrome c_{y} of R. capsulatus (13, FIG. 13). In addition, a vector containing a cleavable, N-terminal signal sequence derived from cytochrome c₂ of R. sphaeroides (15, FIG. 14) was also constructed to enable targeting a soluble foreign protein or the N-terminus of a foreign membrane protein to the periplasmic space of the Rhodobacter host cell.

Platform vectors include affinity tags of altered composition and position. In order to accommodate a target protein whose C-terminus is completely or partially buried, platform vectors with 7-membered histidine tags fused in frame to the N-terminus were constructed. Vectors were constructed in which a site for Tobacco Etch virus (TEV) protease was inserted between the His tag and the start of the foreign gene. Cleavage of the tag results in the addition of three amino acids (SAS) to the N-terminus of the foreign gene. Vectors containing longer C-terminal tags with 10 or 13 consecutive histidines were also constructed and did assist with affinity purification of target proteins, because the longer tags bind more tightly to immobilized metal resin and allow more quantitative removal of impurities that bind either non-specifically or with lower affinity to these columns.

Cloning of genes into the above platform vectors utilizes PCR and ligation methodologies. A vector that enables litigation-independent cloning of genes encoding foreign membrane proteins for expression in R. sphaeroides was designed, constructed, tested and employed in a plate-based automated manner wherein clones were generated in 96-well format utilizing a method based on microtiter plates. The LIC versions of vectors featuring extended tags, N-terminal tags, and cleavable tags have also been designed and/or constructed. FIGS. 2-14 are schematic representations of vectors.

Problematic Soluble Proteins

The Rhodobacter Expression System has been applied more generally to the expression of soluble proteins or multisubunit complexes thereof whose expression has proven to be especially problematic for E. coli-based expression systems. This new soluble protein strategy functions in the absence of the aforementioned membrane protein tether. The Rhodobacter system, in addition to serving as a tool for heterologous expression of membrane proteins, also offers utility for soluble protein expression.

This application of the Rhodobacter Expression System is especially important because large percentage of proteins in current structural genomics efforts (up to and possibly exceeding 50%) are “triaged” when they prove to be expressed at low levels or primarily in insoluble forms in E. coli. Detection of expressed proteins with the anti-polyhistidine antibody has never indicated that expressed proteins form inclusion bodies in Rhodobacter. This is in sharp contrast to T7 polymerase-based E. coli expression systems, where high-level overexpression often results in aggregation and precipitation of incompletely folded polypeptides as inclusion bodies.

Soluble protein expression is accomplished with the same vectors and strategies that have already been used successfully or designed for use with membrane proteins. In an analysis of a wide range of target proteins, the Rhodobacter expression system handles adequately some problematic soluble proteins even IN THE ABSENCE of the membrane protein tether. The only small adaptation of the method is to purify proteins from the cytoplasm and/or periplasm (cell fractions combined as the supernatant from an ultracentrifuge spin at >100,000 g after cell breakage), thereby eliminating the need for solubilization steps or the inclusion of detergents in any chromatographic buffers.

Problematic, supposedly “soluble” proteins that failed in E. coli expression systems, have been produced successfully in soluble form—albeit with reduced yield—in the Rhodobacter system. These target proteins are currently being produced in large scale for crystallization trials.

Because it is capable of successfully expressing soluble proteins which are otherwise lost to inclusion bodies, this suite of expression vectors and hosts forms the basis for a likely “salvage strategy” that will improve the efficiency of existing structural genomics programs. It, thus, expands the versatility of the Rhodobacter Expression System as a vehicle that enables functional and structural studies (and possibly large-scale genomics efforts) for problematic target proteins.

### EXAMPLES

The following examples are illustrative and do not limit the scope of the various methods and compositions disclosed herein.

### Example 1

### Small-Scale Screening for Expression and Localization of Target Protein in Rhodobacter.

For initial expression screening, the cells are grown in small culture volume, and the expression levels and cellular localization of the target protein are determined by Western blotting following SDS-PAGE. Coordinated synthesis of nascent membrane and target membrane protein is autoinduced by decreasing oxygen tension as the cell density increases during semi-aerobic culture. Those conditions are achieved as described below.

### Small-Scale Growth and Preparation of Samples for SDS-PAGE.

### Growth and harvest of expression strains

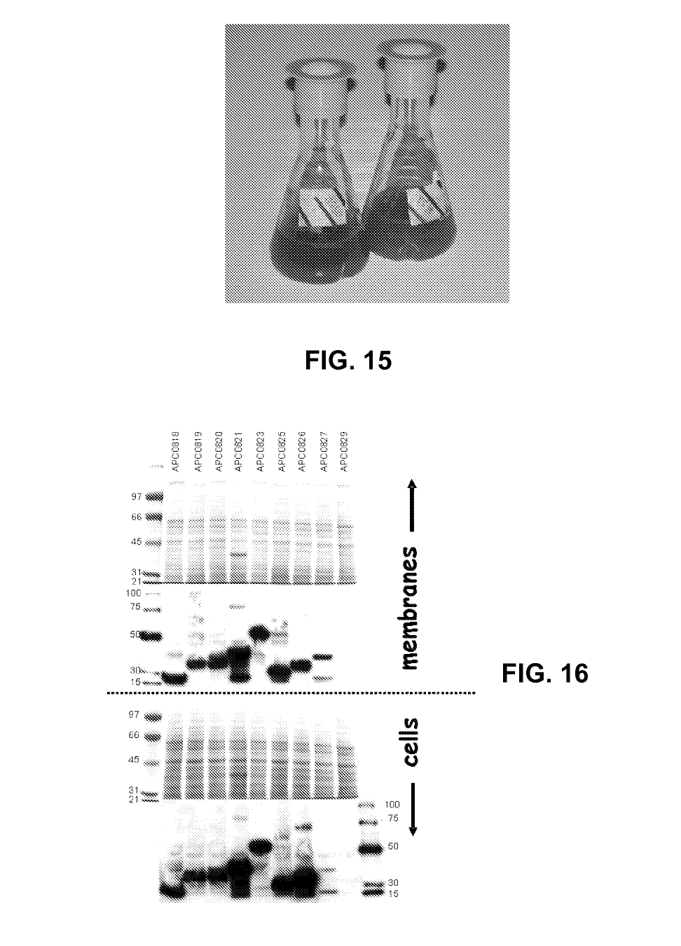

Cells are grown in 80 mL of YCC/tet₁, medium in a 125 mL baffled flask (see FIG. 15). This flask is stoppered with a silicone sponge closure (Bellco Glass, Inc., Cat. No. 2004-00003).

Incubate at 32-34° C., shaking at 125 rpm, for 72-96 hours. Remove 5 mL of media to determine turbidity using a Klett-Summerson calorimeter. The equivalent OD₆₀₀ may also be used. After measurement is complete, refrigerate this sample in a 15 mL Falcon tube for later use in SDS-PAGE. When the Klett value reaches 210-260 or the OD₆₀₀ is ˜2, pellet the remaining 75 mL of cells for 10 minutes at 12,500×g. Discard supernatant and wash cells with 25 mL Buffer 1. Pellet cells as above. Resuspend cells in 25 mL Buffer 1.

### Cell Lysis

Add 300 units of DNase (Sigma D-5025) in 20 μL Buffer 1. Sonicate on ice in a small beaker to disrupt cell aggregates.

Lyse cells in a French press or a microfluidizer at approximately 18,000 psi. Collect into a beaker on ice.

Pellet cell debris for 15 minutes at 22,000×g.

The supernatant is transferred to an ultracentrifuge tube and membranes are pelleted for 45 minutes at 245,000×g. The supernatant is discarded.

Preparing whole cell samples for SDS-PAGE

Pellet the cells from the 5 mL of cells removed previously (for turbidity measurement) for 10 minutes at 12,500×g. Supernatant is decanted and discarded.

Wash the cells once by resuspending and vortexing in 1 mL of Buffer 1. Pellet the cells and discard the supernatant.

Resuspend cells in 150 μL of 0.1 M Tris, pH 8.5, then add 150 μL of Sample Quench. Vortex for 30 seconds.

To shear DNA, sonicate each tube with a microtip probe until foam appears (2-3 seconds).

Place tubes in a 90° C. bath for 10 minutes.

Add 618 μL of TE to each tube.

Vortex each tube for 30 seconds, then heat again at 90° C. for 5 minutes.

Short term storage of tubes is at 4° C. Longer term storage requires freezing at −80° C.

### Preparing Membrane Samples for SDS-PAGE

To each ultracentrifuge tube, add 1 mL of 0.1 M Tris, pH 8.5.

Resuspend the membranes by vortexing, scraping if necessary. A paint brush works very well here. Add 1 mL Sample Quench and mix.

Transfer 1 mL of the resuspended membrane pellet to a microfuge tube and heat for 10 minutes at 90° C.

Vortex this sample for 30 seconds and transfer 100 μL to another microfuge tube.

Save the remaining 900 μL at −20° C. To the 100 μL aliquot, add 206 μL of TE.

Heat each tube for another 5 minutes at 90° C., then vortex for 30 seconds.

Short term storage of tubes is at 4° C. Longer term storage requires freezing at −80° C.

### SDS-PAGE Followed by Electroblotting of Proteins to PVDF Membrane

Replica gels are run in parallel. One gel is stained with Coomassie Brilliant Blue. If heterologously-expressed target proteins are not well-visualized by this method, then proteins of the replica gel are electroblotted to PVDF membrane and the target protein is detected on a Western blot with an anti-polyhistidine antibody.

For SDS-PAGE, assemble gels on apparatus (e.g., Mini-Protean III system from Bio-Rad) with the running buffer required by the gel manufacturer. See gel product manual for the appropriate buffer recipes. Load samples and run gels according to gel manufacturer's specifications.

For gels that will be stained directly, follow these steps: Stain and destain according to instructions from the gel manufacturer and stain/destain manufacturer.

For gels that are to be electroblotted to PVDF membranes for Western blots, follow these steps: Prepare adequate quantities of the blotting buffer “TGMS”. This is a 1×solution that is prepared by dilution of 10×blotting buffer. Approximately 1 L of TGMS is required per electrotransfer tank.

While the gel is running, prepare the PVDF membrane for transfer by first wetting in a minimal amount of methanol, then placing it in 50 mL TGMS for further wetting (with rocking).

When SDS-PAGE is complete, disassemble the plates and remove the gel. Soak it in 50 mL TGMS for only five minutes, with rocking. This short soak ensures that some SDS remains to prevent membrane proteins from precipitating in the gel.

Assemble the blotting sandwich according to directions provided by the manufacturer.

Be sure that all air bubbles are removed, especially between the gel and the PVDF membrane. Everything should be thoroughly wetted in TGMS at the time of assembly.

Transfer the blotting sandwich to the blotting tank filled with TGMS. Place a small stir bar in the bottom of the tank and use an ice reservoir to keep the initial transfer cold.

Transfer at 300 mA (1 hour), then overnight at 100 mA with slow stirring.

Disassembly and waste disposal: Separate the sandwich layer by layer, taking care to note the orientation of the PVDF membrane, and place it in container with protein side up. Either proceed immediately to development of the Western blot or allow the PVDF membrane to air dry for later processing. If the PVDF is allowed to dry, it must be wetted again in methanol prior to transfer to any aqueous solution for further processing.

Stain the electroblotted gel to determine transfer efficiency. Dispose of the tank blotting buffer in a hazardous waste container. Western Blot Development Using an Anti-polyhistidine Antibody. (Protocol adapted from those found at www.novagen.com and www.piercenet.com).

Resuspension of His·Tag Monoclonal Antibody: The His·Tag Monoclonal Antibody (Novagen 70796-3) is provided as a lyophilized powder and must be resuspended prior to use in the following protocols. Dissolve the lyophilized antibody in 500 sterile water per 100 μg vial or 15 μL sterile water per 3 μg vial (final concentration 0.2 mg/mL).

Chemiluminescent detection: Alkali-soluble Casein (Novagen 70955-3; stored at 4EC) is the recommended blocking reagent for chemiluminescent detection on nitrocellulose membranes because it results in the lowest background and can be used as a blocking reagent throughout the protocol. The following conditions work well for the hydrophobic PVDF blotting membranes recommended. Note that different membranes may require different blocking conditions (e.g. longer blocking incubations, higher concentration of blocking reagent).

Reagent Preparation: Prepare 30 mL of blocking solution (1% Alkali-soluble casein in 1×TBS) per blot by mixing 6 mL of 5% Alkali-soluble Casein with 24 mL of deionized water. Fresh blocking solution should be prepared each time. Reserve the blocking solution throughout the procedure because it will also be used for the primary and secondary antibody dilution.

Prepare 1 L each of 1×TBS and 1×TBSTT. They may be prepared by diluting 10×stocks. Filter sterilize the 1×TBSTT.

The resuspended His·Tag Monoclonal 1E Antibody will be used at a dilution of 1:1000 in blocking solution (7.5 μL in 7.5 mL total).

The Goat Anti-Mouse IgG HRP conjugate 2Eantibody (Novagen 71045-3) will be used at a dilution of 1:50,000 in blocking solution. Total volume is 20 mL for this step.

Development of the Western blot: The following steps should be performed at room temperature, with gentle agitation or rocking during incubations. For the standard 5.5 cm×8.5 cm pieces of PVDF that fit purchased mini-gels, use clear plastic 6.5 cm×9 cm trays for all incubations. Place the membrane in the tray with the protein-side up, as determined by marking it or using colored molecular weight standards. The solution volumes used in this protocol are based on a 5.5 cm×8.5 cm blot. Larger or smaller membranes will require adjustment of the volumes.

If starting with a dried PVDF membrane, first re-wet it by soaking in methanol. Transfer the membrane to 15 mL 1×TBS and perform two washes, each of 10 minutes.

Discard the washes and incubate the membrane in 15 mL blocking solution for at least 1 hour. Remove 7.5 mL of blocking solution from the tray and save it for later. To the remaining 7.5 mL of blocking solution in the tray, add 7.5 μL of the His·Tag Monoclonal 1E Antibody (thus diluted 1:1000) and incubate for 1 hour with rocking.

Wash twice, for 10 minutes each time, with 20 mL 1×TBSTT to remove unbound 1E antibody.

Wash for 10 minutes with 15 mL 1×TBS. Incubate for 1 hour with 20 mL Goat anti-Mouse IgG HRP Conjugate 2E antibody diluted 1:50,000 in blocking solution (see Reagent Preparation, step 4, above).

Wash at least five times, for 10 minutes each wash, using 20 mL 1×TBSTT per wash. It is important to thoroughly wash the membrane at this point to achieve maximum signal:noise ratios.

After the final washing step is complete, drain as much TBSTT from the membrane as possible.

Addition of the substrate: For a typical 5.5 cm×8.5 cm membrane, use 3 mL each of Pierce Pico peroxide solution (#1856135) and Pierce Pico enhancer (#1856136), and 0.25 mL each of Pierce Dura peroxide solution (#1856158) and Pierce Dura enhancer (#1856157) for a total volume of 6.5 mL. Incubate the membrane in the substrate at room temperature for 5 minutes with rocking.

Remove the membrane from the substrate. Drain any excess substrate from the membrane by touching the edge to a paper towel. Place the membrane in a clear plastic development folder and fold the plastic over the membrane. Remove any bubbles between the plastic and the membrane. Gently remove any liquid from the exterior of the plastic.

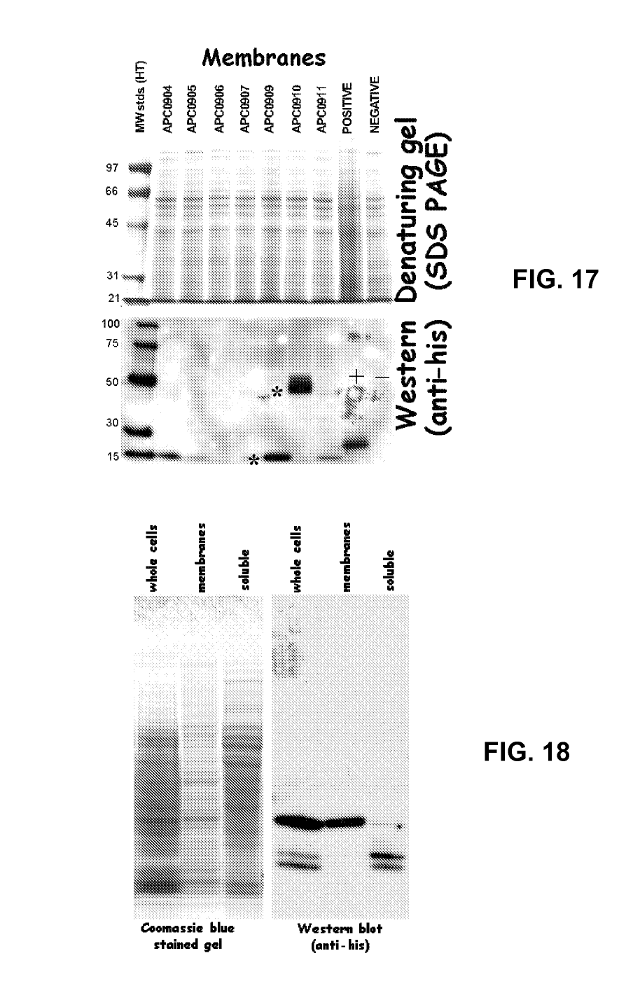

Use the membrane to expose x-ray film for identification of expressed target proteins. Typical results from a screening experiment of this type using the pRKPLHT1Dpuf (Table 1) expression vector are shown in FIG. 16. Expression yield can be crudely estimated as outlined in FIG. 17 (again shown here for expression vector pRKPLHT1Dpuf).

Differential centrifugation may be used to determine the cellular localization of the expressed target membrane protein in Rhodobacter cells (e.g., FIG. 16). Expression in whole cells is compared (on an equal volume basis using Western analysis with an anti-polyhistidine antibody) with the supernatant (soluble fraction) and pellet (membrane fraction) obtained from ultracentrifuge separation (245,000×g) of lysates that are devoid of cellular debris. Most of the target membrane proteins that have been studied are expressed predominantly in the Rhodobacter ICM. Very few target membrane proteins show any significant presence in the soluble fraction. The sum of the signals from the soluble and membrane fractions should equal the total expression level observed in the cells. If this is not the case, one should investigate the debris pellet obtained from the lysate to test for the presence of target protein that may have aggregated as inclusion bodies—a phenomenon not yet observed with the expression of membrane proteins in Rhodobacter.

### Example 2

### Summary of Results with Platform Vector pRKPLICHT1Dpuf.

Expression Analysis with recently designed and constructed vector pRKLICHT1Dpuf.

The ligation-independent-cloning vector pRKLICHT1Dpuf was initially tested with target genes that were characterized by good expression using pRKPLHT1Dpuf. These prokaryotic membrane protein genes were numbered APC00809, APC00821, and APC00951. Expression analysis in whole cells, crude membrane preparations, and the soluble fraction are shown in FIG. 17. No differences in expression levels are apparent when comparing results from the same gene expressed from pRKPLHT1Dpuf or pRKLICHT1Dpuf.

Membrane proteins from E. coli that have no known homolog in the PDB are selected for expression. If a Rhodobacter homolog of the E. coli target exists, then it is also selected. Information obtained from a single structure by focusing on large protein families is maximized. Targets exhibiting a wide range of MW, pIs, and hydropathy plot signatures are selected intitially.

A typical set of oligonucleotides used to amplify and subsequently clone a target membrane protein gene (APC00809) into pRKPLHT1Dpuf is shown in FIG. 22. This success spawned the use of pRKLICHT1Dpuf for semi-automated cloning of 288 membrane protein genes (from E. coli and B. subtilis).

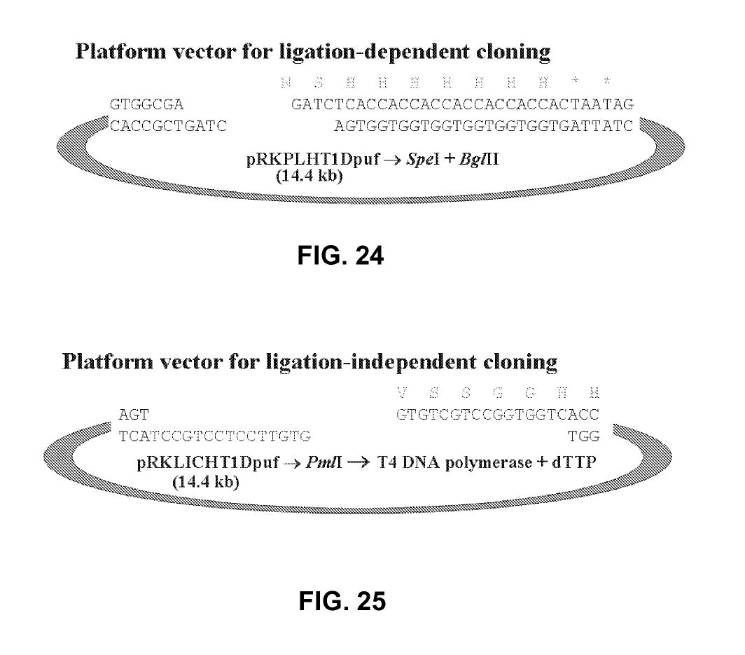

A set of oligonucleotides (FIG. 23) was used to amplify and subsequently clone a target membrane protein gene (APC00809) into pRKLICHT1Dpuf. FIGS. 24-25 show strategy for cloning target membrane protein genes in to versatile vectors.

### Example 3

### Adapatability of Versatile Vectors to Various Photosynthetic Bacteria

Several vectors disclosed herein, for example in Tables 1 and 2, can be adapted for use in other bacterial species using methodology known to a skilled artisan. In some of the embodiments disclosed herein, the pufB and pufA genes (B and A subunits) of the light-harvesting I complex (LHI) of Rhodobacter sphaeroides were replaced with a gene of interest. The gene of interest is flanked at the C-terminal or N-terminal end by an affinity tag and may be followed by a protease digestion site. Similarly, a skilled artisan can clone an appropriate, functionally similar operon from another bacterial species to replace the puf or puc operon backbone present in some of the vectors disclosed herein. Host genes that are not essential in membrane formation, membrane integrity or survival of host bacteria, may be replaced with a gene of interest under an appropriate promoter to obtain a suitable level of expression. The N-terminal or C-terminal affinity tags as part of the vector backbone can be used in the design of vectors capable of multiplication in a traditional host such as E. coli and are also capable of expressing a desired gene in a photosynthetic bacteria such as, for example, Rhodopseudomonas, Rhodocyclus, and Chlorobium.

Coordinately, it is desirable to delete chromosomal copies of the non-essential host gene whose plasmid-borne copy is being replaced by the foreign gene, thus engineering added capacity in the host membranes for accommodating over-expressed heterologous foreign membrane proteins.

Some commercially applicable target membrane proteins that can be expressed using the vectors disclosed herein include receptors including G-protein coupled receptors, ion channels, transporters, membrane-bound enzymes, cytoskeletal membrane proteins, and membrane proteins specific to prokaryotic pathogens.

### Example 4

### Production of a Soluble Protein in Rhodobacter that Proved Problematic when Expressed in E. coli.

When expression of ILR1, a soluble protein derived from Arabidopsis thaliana, was attempted in E. coli using an expression system based upon T7 polymerase, the expressed protein aggregated and precipitated in a non-functional state in the form of inclusion bodies. To test whether this problem could be circumvented by features of the Rhodobacter expression system, this gene was cloned into pRKPLHT1Dpuf for expression in R. sphaeroide. Results from small-scale screening suggest that the protein associates with the ICM and that the elaboration of additional membranes in Rhodobacter allows for successful expression of this protein in an unaggregated state (FIG. 20). Rhodobacter produced this protein at an approximate level of 2 mg protein per liter of cell culture (sufficient for subsequent larger-scale purification efforts).

TABLE 1
Versatile Vectors designed for use in Rhodobacter Expression System
Vector Properties

| | Clon- | | | |
|---|---|---|---|---|
| | ing | Tag | | |
| | Strat- | Posi- | Tag | Cleavable |
| Vector Name | egy | tion | Length | Components^{¥} |
| pRKMALICHT1Dpuf | LIC | C | 7 | none |
| pRKMALICHT10Dpuf | LIC | C | 10 | none |
| pRKMALICHT13Dpuf | LIC | C | 13 | none |
| pRKHTMALIC1Dpuf | LIC | N | 7 | none |
| pRK10HTMAPLIC1Dpuf | LIC | N | 10 | none |
| pRK13HTMALIC1Dpuf | LIC | N | 13 | none |
| pRKMATEVLICHT1Dpuf | LIC | C | 7 | membrane anchor |
| pRKMATEVLICHT10Dpuf | LIC | C | 10 | membrane anchor |
| pRKMATEVLICHT13Dpuf | LIC | C | 13 | membrane anchor |
| pRKMALICTEVHT1Dpuf | LIC | C | 7 | tag |
| pRKMALICTEVHT10Dpuf | LIC | C | 10 | tag |
| pRKMALICTEVHT13Dpuf | LIC | C | 13 | tag |
| pRKHTTEVMALIC1Dpuf | LIC | N | 7 | tag |
| pRK10HTTEVMALIC1Dpuf | LIC | N | 10 | tag |
| pRK13HTTEVMALIC1Dpuf | LIC | N | 13 | tag |
| pRKHTMATEVLIC1Dpuf | LIC | N | 7 | tag and anchor |
| pRK10HTMATEVLIC1Dpuf | LIC | N | 10 | tag and anchor |
| pRK13HTMATEVLIC1Dpuf | LIC | N | 13 | tag and anchor |
| pRKSSLICHT1Dpuf | LIC | C | 7 | none |
| pRKSSLICHT10Dpuf | LIC | C | 10 | none |
| pRKSSLICHT13Dpuf | LIC | C | 13 | none |
| pRKSSLICTEVHT1Dpuf | LIC | C | 7 | tag |
| pRKSSLICTEVHT10Dpuf | LIC | C | 10 | tag |
| pRKSSLICTEVHT13Dpuf | LIC | C | 13 | tag |
| pRKSSHTLIC1Dpuf | LIC | N | 7 | none |
| pRKSS10HTLIC1Dpuf | LIC | N | 10 | none |
| pRKSS13HTLIC1Dpuf | LIC | N | 13 | none |
| pRKSSHTTEVLIC1Dpuf | LIC | N | 7 | tag |
| pRKSS10HTTEVLIC1Dpuf | LIC | N | 10 | tag |
| pRKSS13HTTEVLIC1Dpuf | LIC | N | 13 | tag |

^{¥}The signal sequence, by definition, is cleaved and not denoted as such here.
Definitions:
LDC = ligation dependent cloning
LIC = ligation independent cloning
HT = polyhistidine tag
TEV = tobacco etch virus
MA = membrane anchor
SS = signal sequence

TABLE 2
Description of cloning sites and affinity tags in the expression vectors

| Vector name | DNA sequence position | Site Description |
|---|---|---|
| pRKHTLIC1Dpuf | 2563-2565 | ATG start site (underlined) |
| (N-terminal 7 x His tag | 2566-2586 | 7x Histidine tag (gray) |
| for ligation-independent | 2601-2606 | SnaBI restriction site (bold) |
| cloning) | | |
| pRKHTPL1Dpuf | 2563-2565 | ATG start site (underlined) |
| (N-terminal 7 x His | 2566-2586 | 7x Histidine tag (gray) |
| tag) | 2587-2592 | NheI restriction site (bold) |
| | 2599-2604 | BglII restriction site (bold) |
| pRKHTTEVLIC1Dpuf | 2563-2565 | ATG start site (underlined) |
| (N-terminal 7 x His tag | 2566-2586 | 7x Histidine tag (gray) |
| followed by cleavage site for | 2587-2607 | TEV protease recognition site |
| Tobacco Etch Virus protease | 2610-2615 | (double underlined) |
| for ligation-independent | | SnaBI restriction site (bold) |
| cloning) | | |
| pRKHTTEVPL1Dpuf | 2563-2565 | ATG start site (underlined) |
| (N-terminal 7 x His tag | 2566-2586 | 7x Histidine tag (gray) |
| followed by cleavage site for | 2587-2607 | TEV protease recognition site |
| Tobacco Etch Virus protease) | 2608-2613 | (double underlined) |
| | 2620-2625 | NheI restriction site (bold) |
| | | BglII restriction site (bold) |
| pRKLICHT10Dpuf | 2562-2567 | PmlI restriction site (bold) |
| (C-terminal 10 x His | 2580-2609 | 10x Histidine tag (gray) |
| tag for ligation-independent | 2610-2615 | Stop codons (italicized) |
| cloning) | | |
| pRKLICHT13Dpuf | 2562-2567 | PmlI restriction site (bold) |
| (C-terminal 13 x His | 2580-2618 | 13x Histidine tag (gray) |
| tag for ligation-independent | 2619-2623 | Stop codons (italicized) |
| cloning) | | |
| pRKLICHT1Dpuf | 2562-2567 | PmlI restriction site (bold) |
| (C-terminal 7 x His tag | 2583-2600 | 7x Histidine tag (gray) |
| for ligation-independent | 2601-2605 | Stop codons (italicized) |
| cloning) | | |
| pRKPLHT10Dpuf | 2564-2569 | SpeI restriction site (bold) |
| (C-terminal 10 x His | 2572-2577 | BglII restriction site (bold) |
| tag) | 2578-2607 | 10x Histidine tag (gray) |
| | 2608-2613 | Stop codons (italicized) |
| pRKPLHT13Dpuf | 2564-2569 | SpeI restriction site (bold) |
| (C-terminal 13 x His | 2572-2577 | BglII restriction site (bold) |
| tag) | 2578-2616 | 13x Histidine tag (gray) |
| | 2617-2622 | Stop codons (italicized) |
| pRKMAHT1Dpuf | 2572-2719 | Membrane anchor/linker domain |
| (N-terminal membrane | 2720-2725 | SpeI restriction site (bold) |
| anchor/linker domain; C- | 2736-2741 | BglII restriction site (bold) |
| terminal 7 x His tag) | 2742-2762 | 7 x Histidine tag (gray) |
| | 2763-2768 | Stop codons (italicized) |
| pRKSSHT1Dpuf | 2639-2644 | SpeI restriction site (bold) |
| (N-terminal signal | 2655-2660 | BglII restriction site (bold) |
| sequence; C-terminal 7 x His | 2573-2638 | Signal sequence (underlined) |
| tag) | 2661-2681 | 7 x Histidine tag (gray) |
| | 2682-2687 | Stop codons (italicized) |

### MATERIALS AND METHODS

Vector Construction

Construction of pRKHTPL1Dpuf: The small EcoRI-HindIII fragment of plasmid pRKHTMHBgl [11] was subcloned into pBluescript SK+(Stratagene, Inc.). The insert of the resulting plasmid was digested with NspI and FseI to excise the pufB and pufA genes encoding subunits of the light-harvesting I antennae complex. That fragment was replaced by a synthetic oligonucleotide cassette:

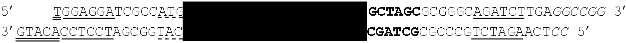

encoding an NspI site (double underlined), an ATG start codon (dashed underline), a 7×Histidine tag (CAC, gray), an FseI site (italics), and unique NheI (bold) and BglII (underlined) sites for cloning of foreign genes. This modified region was then excised as an EcoRI-ClaI fragment and was swapped for the existing EcoRI-ClaI fragment of pRKPLHT1Dpuf.

Construction of pRKHTTEVPL1Dpuf: The procedure was the same as that used for the construction of pRKHTPL1Dpuf above, with the exception that the synthetic oligonucleotide cassette used was:

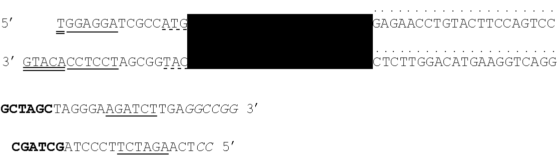

encoding an NspI site (double underlined), a ribosome binding site (thick underline), an ATG start codon (dashed underline), a 7×Histidine tag (CAC, black), a segment encoding the recognition site for the protease from Tobacco Etch Virus (TEV;

dotted emphasis) an FseI site (italics), and unique NheI (bold) and BglII (underlined) sites for cloning of foreign genes. This modified region was then excised as an EcoRI-ClaI fragment and was swapped for the existing EcoRI-ClaI fragment of pRKPLHT1Dpuf.

Construction of pRKPLHT10Dpuf: The small EcoRI-HindIII fragment of plasmid pRKHTMHBgl was subcloned into pBS+(Stratagene, Inc.). The insert of the resulting plasmid was digested with NspI and FseI to excise the pufB and pufA genes encoding subunits of the light-harvesting I antennae complex. That fragment was replaced by a synthetic oligonucleotide cassette:

5′-CTAGTTCCA [symbol] TGATAGATCTCACCACCACCACCACCACCACCACCA
CCACTAATAGGCCGG-3′
AAGGTATACTATC [symbol] GAGTGGTGGTGGTGGTGGTGGTGGTGGTGGTGATT
ATCC-3′

encoding an NspI site (blue), an ATG start codon (pink), a 7×Histidine tag (CAC, black), an FseI site (orange), and unique NheI (teal) and BglII (purple) sites for cloning of foreign genes. This modified region was then excised as an EcoRI-ClaI fragment and was swapped for the existing EcoRI-ClaI fragment of pRKPLHT1Dpuf.

Construction of pRKPLHT13Dpuf: The procedure was the same as that used for the construction of pRKPLHT10Dpuf above, with the exception that the synthetic oligonucleotide cassette used contained codons for 13 histidine residues instead of 10.

Construction of pRKLICHT1Dpuf: The small EcoRI-HindIII fragment of plasmid pRKPLHT1Dpuf was subcloned into pBluescript SK+(Stratagene, Inc.). The insert of the resulting plasmid was digested with PflMI and BglII to excise the multiple cloning site region of this expression vector. The DNA was then treated with mung bean nuclease to generate blunt ends, then the plasmid was treated with T4 DNA polymerase in the presence of dTTP to generate the desired overhangs that were complementary to the following synthetic oligonucleotide cassette:

5′-AACCCACGCCACCAGTAGGCAGGAGGAACACGTGTCGTCCGGTGG-
3′
3′-ATCCGTCCTCCTTGTGCACAGCAGGCCACCAG-5′

This cassette encodes a ribosome binding site (magenta) and a PmlI site (dark red), and it was annealed to the modified plasmid. This modified cloning region was then excised as an EcoRI-ClaI fragment and was swapped for the existing EcoRI-ClaI fragment in a version of pRKPLHT1Dpuf in which an existing PmlI site had been repaired. The unique PmlI site located in the cloning site region of the resulting expression vector pRKLICHT1Dpuf facilitates the linearization of the plasmid prior to treatment with T4 polymerase to generate overhangs for ligation-independent cloning.

Construction of pRKLICHT10Dpuf: This plasmid was constructed in a manner analogous to the construction of pRKLICHT1Dpuf above, with the exception that the initial manipulations were performed on a plasmid that contained the small EcoRI-HindIII fragment of plasmid pRKPLHT10Dpuf subcloned into pBluescript SK+(Stratagene, Inc.).

Construction of pRKLICHT13Dpuf: This plasmid was constructed in a manner analogous to the construction of pRKLICHT1Dpuf above, with the exception that the initial manipulations were performed on a plasmid that contained the small EcoRI-HindIII fragment of plasmid pRKPLHT13Dpuf subcloned into pBluescript SK+(Stratagene, Inc.)

Construction of pRKHTLIC1Dpuf: Expression vector pRKHTPL1Dpuf was digested with NheI and BglII to excise the cloning site region of the plasmid. The DNA was then treated with mung bean nuclease to generate blunt ends, then the plasmid was treated with T4 DNA polymerase in the presence of dCTP to generate overhangs that were complementary to a synthetic oligonucleotide cassette:

5′-GCCTATTCCAATCCTACGTAGAAGGGAAGATC-3′
3′-GGATAAGGTTAGGATGCATCTTCCCTTCTAGAA-5′

The cassette includes a unique SnaBI site (dark green) that facilitates the linearization of the plasmid prior to treatment with T4 polymerase to generate overhangs for ligation-independent cloning.

Construction of pRKHTTEVLIC1Dpuf: Expression vector pRKHTPL1Dpuf was digested with NheI and BglII to excise the cloning site region of the plasmid. The DNA was then treated with mung bean nuclease to generate blunt ends, then the plasmid was treated with T4 DNA polymerase in the presence of dCTP to generate overhangs that were complementary to a synthetic oligonucleotide cassette:

5′-GAGAACCTGTACTTCCAATCCTTTACGTAGAAATAGGGAAGATC-
3′
3′-TCTTGGACATGAAGGTTAGGAAATGCATCTTTATCCCTTCTAGAA-
5′

The cassette includes a region that encodes a recognition site for the TEV protease and a unique SnaBI site (dark green) that facilitates the linearization of the plasmid prior to treatment with T4 polymerase to generate overhangs for ligation-independent cloning.

Design of Oligonucleotide Primers for Gene Amplification and Cloning.

The choice of cloning strategy (ligation-dependent or ligation-independent) and specific vector (N- or C-terminal tag; protease site, etc.) will dictate the composition of the oligonucleotides used to amplify the target gene. It is also important to determine whether there are any codons in the first 50 that are extremely rare in Rhodobacter (e.g., TTA). If so, one may want to consider cloning a paralog or homolog that lacks rare codons. For a higher-throughput approach to designing sets of oligonucleotides for the cloning of multiple target genes, primer generator tools [10] can be used instead of relying on manual design. Two examples include one for ligation-dependent cloning and the other for ligation-independent cloning, both using vectors with C-terminal, non-cleavable polyhistidine tags—of typical oligonucleotide design for amplification of target genes to be compatible with one of the platform vectors of the Rhodobacter system.

Ligation-Dependent Cloning Using pRKPLHT1Dpuf.

For ligation-dependent cloning, decide which enzymes will be used to insert the gene into the expression vector, choosing those enzymes for which there are no sites in the target gene. The SpeI site in pRKPLHT1Dpuf is also compatible with XbaI and AvrII overhangs. The BglII site in pRKPLHT1Dpuf is compatible with BamHI and BclI. Note that combining the BclI overhang with that of BglII produces an in-frame TGA stop codon; this may be desirable if the preference is to express the target protein without the C-terminal polyhistidine tag.

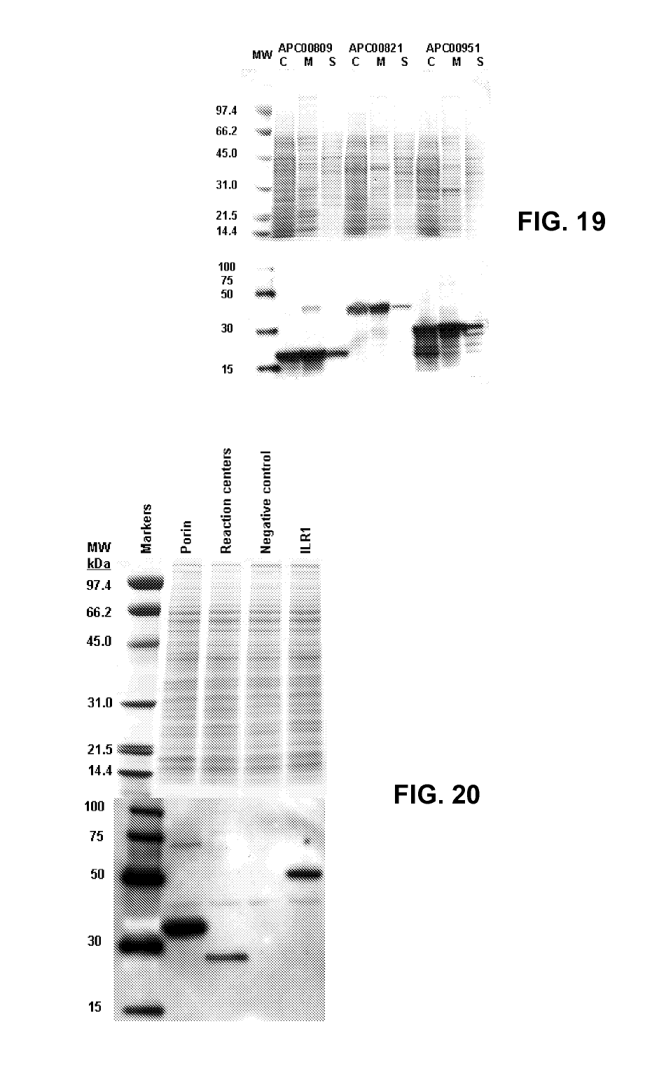

Typical 5′- and 3′-oligonucleotides (“top” and “bottom”, respectively) are shown in FIG. 19. Four to six “dummy” bases are included at the 5′-end of each oligonucleotide to enable efficient digestion of the amplicon by the restriction enzyme. This sequence is followed in the top primer by the restriction site sequence and a ribosome binding site (Rhodobacter RBS=GGAGG) placed 4-12 bases before the start codon; typically, the RBS is placed six bases before the start codon. The bottom primer incorporates the sequence for the second restriction enzyme site followed by the gene sequence. A polyhistidine tag and stop codons are encoded by the platform vectors, thus the native stop codon of the target gene should not be included in the amplicon. Oligonucleotides should be designed such that they have good GC-clamps at the 3′ ends; at least three contiguous Gs or Cs are recommended.

Using any standard software, examine the oligonucleotide sequences to determine the melting temperature of the complementary region for use in determining annealing temperature for PCR reactions. Tₘs of the complementary regions of the primer sets should match within 5° C.

Ligation-Independent Cloning Using pRKLICHT1Dpuf

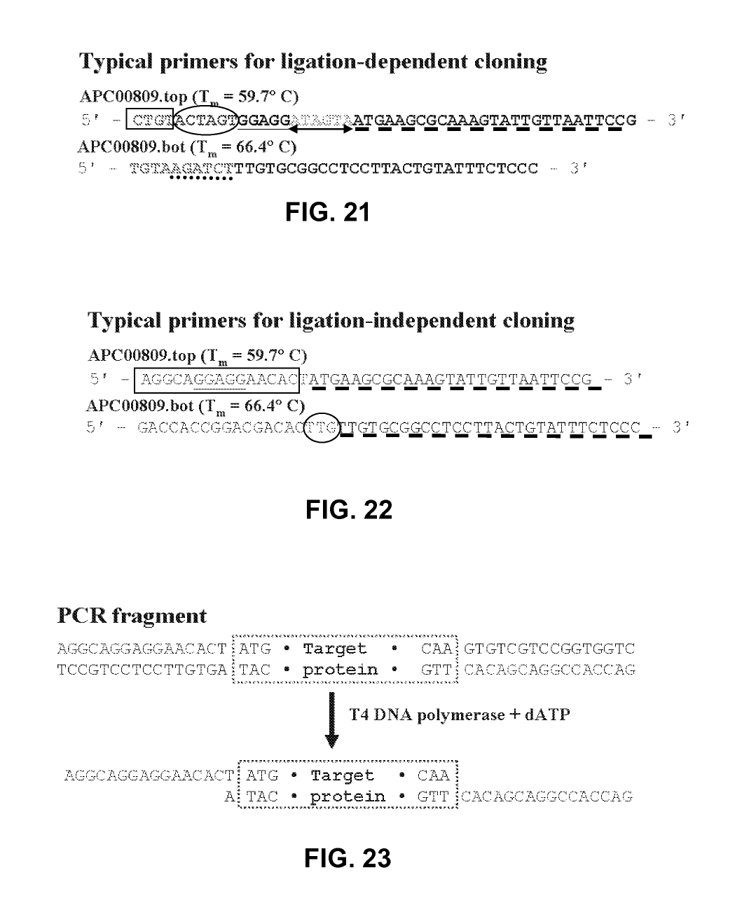

Typical 5′- and 3′-oligonucleotides for use in ligation-independent cloning of a target gene are shown in FIG. 20. The 5′-end of the top primer begins with the sequence that provides a LIC overhang which is complementary to that of the platform vector (FIG. 21), followed by the RBS placed 4-12 bases before the start codon; typically, the RBS is placed six bases before the start codon. The 5′-end of the bottom primer begins with the other complementary LIC overhang, followed by the gene sequence. A polyhistidine tag and stop codons are encoded by the platform vectors, thus the native stop codon of the target gene should not be included in the amplicon. Oligonucleotides should be designed such that they have good GC-clamps at the 3′ ends; at least three contiguous Gs or Cs are recommended.

Using any standard software, examine the oligonucleotide sequences to determine the melting temperature of the complementary region and to check for regions of stable secondary structure.

Platform Vector Preparation

In order to prepare the platform vectors for ligation-dependent (pRKPLHT1Dpuf) or ligation-independent (pRKPLICHT1Dpuf) cloning, steps are provided herein by which the relatively large vectors are linearized and compatible, cohesive ends are generated. The protocols for ligation-dependent cloning that use restriction enzymes are outlined separately from the protocols for ligation-independent cloning that use the proof-reading exonuclease activity of T4 DNA polymerase. Examples include one for ligation-dependent cloning and the other for ligation-independent cloning, both using vectors with C-terminal, non-cleavable polyhistidine tags—that prepare platform vectors for insertion of foreign genes for expression in the Rhodobacter system. Similar steps are used in the preparation of other vectors (Table 1) described herein.

Large-Scale Vector Preparation Protocol for Ligation-Dependent Cloning using pRKPLHT1Dpuf

The platform vector, pRKPLHT1Dpuf, used for ligation-dependent cloning (FIG. 22) has a simple multiple cloning site with three unique restriction sites (SpeI, NdeI, and BglII) for target gene insertion. Routine cloning has been achieved using SpeI and BglII, and protocols below are designed and written based on the assumption that these restriction endonucleases will be utilized.

The conditions that work well for small reactions do not scale well to large volumes, thus multiple small reactions are preferred to one larger reaction to keep background levels of uncut plasmid low. Set up multiple tubes using this protocol to generate a large supply of digested vector. Typically, 3-4 reactions are good since this yields 150+μL of cloning vector, which is adequate for the cloning of approximately 150 target genes. Allow the reaction to incubate at 37° C. for at least 2 hours to ensure complete digestion.
Preparatory Digestion:
25 μL pRKPLHT1Dpuf
2.5 μL SpeI
2.5 μL BglII
8 μL Promega buffer B
42 μL sterile ddH₂0
For a total volume of 80 μL

When using plasmid DNA prepared with basic alkaline lysis miniprep protocols, RNase should be included in the reaction. Most modern miniprep kits employ RNase during cell lysis and, hence, RNase can be excluded from the typical restriction endonuclease reaction, as presented above. The preparatory digest above assumes that the concentration of the plasmid DNA stock is between 0.3 and 2 μg/μL.

When preparing to gel purify the DNA fragments, pour an 0.8% agarose gel and use a preparative comb.

Run the gel for at least 1.5 hours at 60 volts to help determine if the digestion was complete and to be able to separate linear from circular uncut plasmid DNA and then excise the band.

Purification and Evaluation of Digested Vector:

Extract the DNA from the excised agarose slice using a commercially available gel extraction kit (e.g., MoBio UltraClean GelSpin kit). Use a maximum of 0.2 g minced agarose per spin filter.

The QiaEx II does have a greater recovery rate by about 40-50%, however it is very time consuming (over an hour). Recovery from the MoBio kit is much quicker (˜7 minutes) and the yield is lower. MoBio kit is satisfactory.

Before using the digested vector in an experimental reaction, run a control ligation (no insert) to determine background of colonies resulting from contamination of it by uncut or singly-cut vector. Store the digested vector at 4° C.

Large-Scale Vector Preparation Protocol for Ligation-Independent Cloning using pRKLICHT1Dpuf.

To generate the LIC overhangs, platform vector pRKLICHT1Dpuf is first linearized by digestion with PmlI and then treated with T4 DNA polymerase in the presence of dTTP. The exonuclease activity of the polymerase yields the overhangs that are shown in red in FIG. 23.

Since conditions that work well for small reactions often do not scale well to large volumes, the best results are achieved when multiple small reactions are performed and then combined following enzymatic digestion. The following steps indicate amounts of DNA used in typical preparations of vector carrying the LIC overhangs.

Vector linearization with PmlI: Digest 10 μL pRKLICHT1Dpuf (1/5 of the yield of plasmid DNA from a standard miniprep protocol) with PmlI in 70 μL reaction volume for one hour at 37° C. PmlI is an unstable enzyme and best results are achieved by adding a second aliquot half-way through the incubation. Clean up the reaction with any standard purification kit that is suitable for plasmids larger than 10 kb.

Generation of LIC overhangs: One half of the PmlI-digested DNA should be used in generating the sticky ends with LIC-qualified T4 DNA polymerase.
pRKLICHT 1 Dpuf/PmlI
1 μL 100 mM dTTP
2 μL 100 mM DTT
4 μL 10×T4 polymerase reaction buffer
1 unit T4 DNA polymerase
Total volume of 40 μL

Incubate at room temperature for 30 minutes, then inactivate the polymerase at 75° C. for 20 minutes. This inactivated mixture can be used directly in annealing reactions or it can be cleaned up using a standard purification kit that is suitable for plasmids larger than 10 kb.

Before using the digested vector in an experimental reaction, determine the background of colonies resulting from contamination of it by undigested vector. Store the digested vector at 4° C.

Vector Sequences

Broad-host-range expression vector with N-terminal 7 ± His tag for Ligation
Independent Cloning
pRKHTLIC1Dpuf.seq Length: 14386

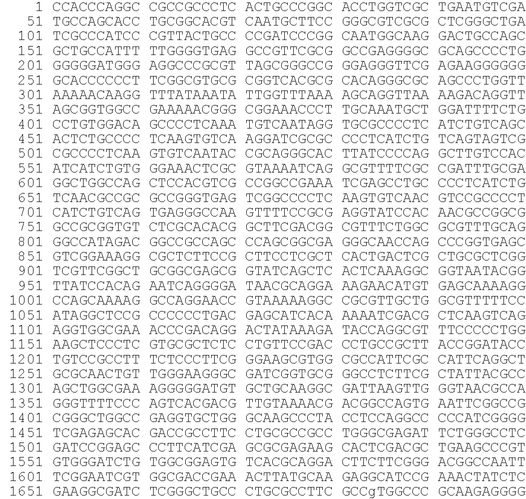

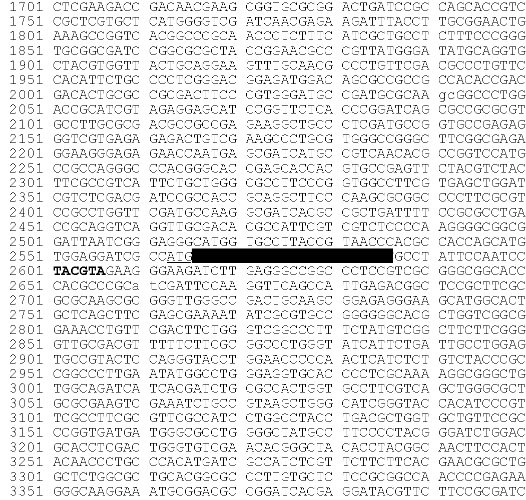

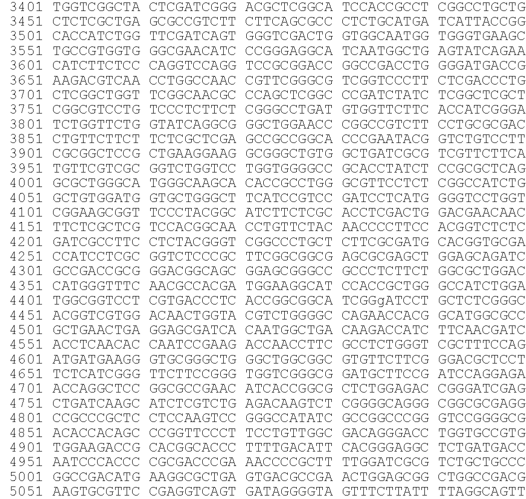

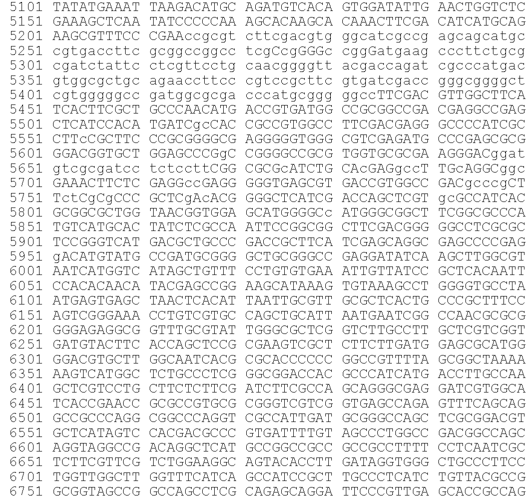

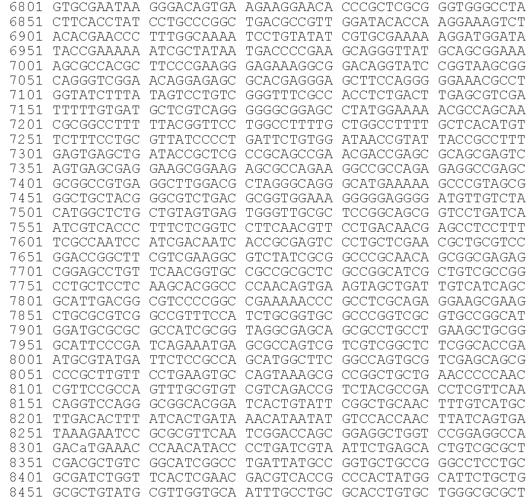

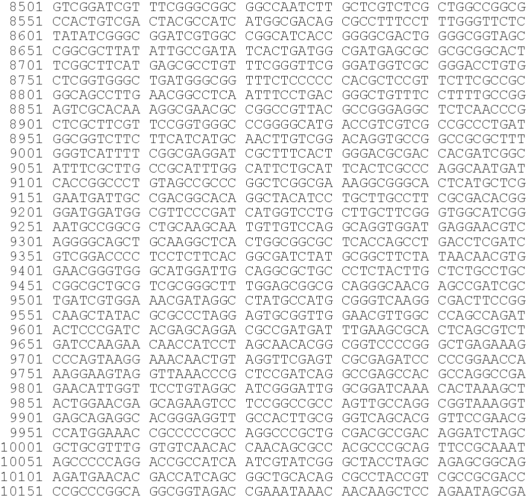

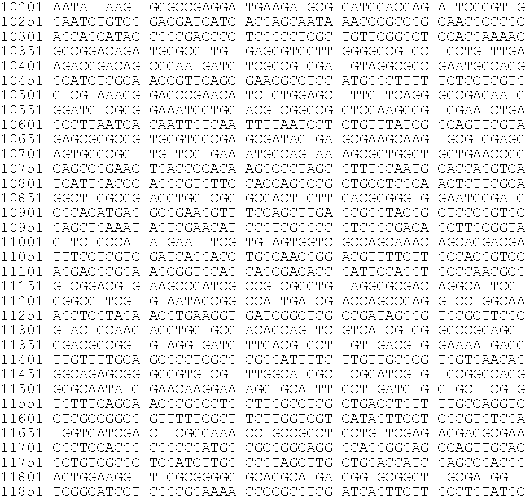

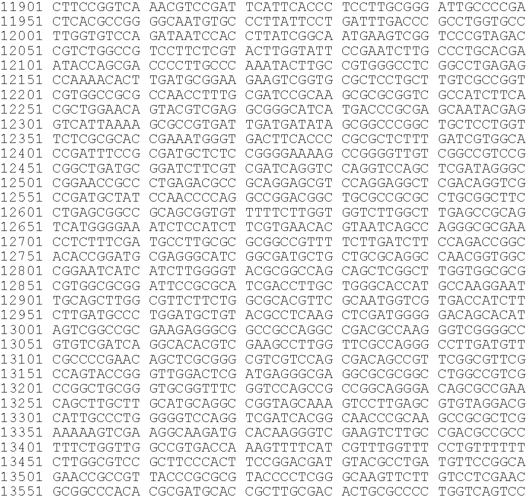

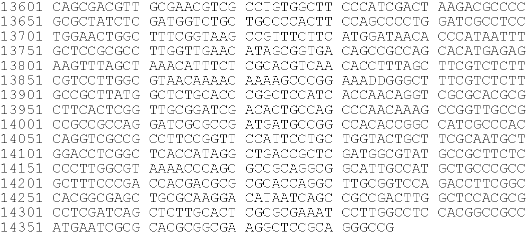

Broad-host-range expression vector with N-terminal 7 ± His tag
pRKHTPL1Dpuf.seq Length: 14370

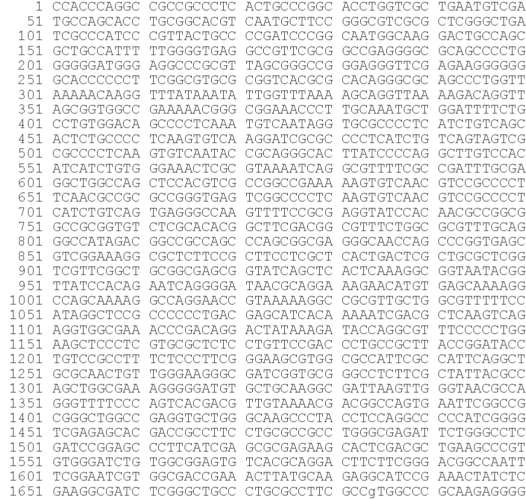

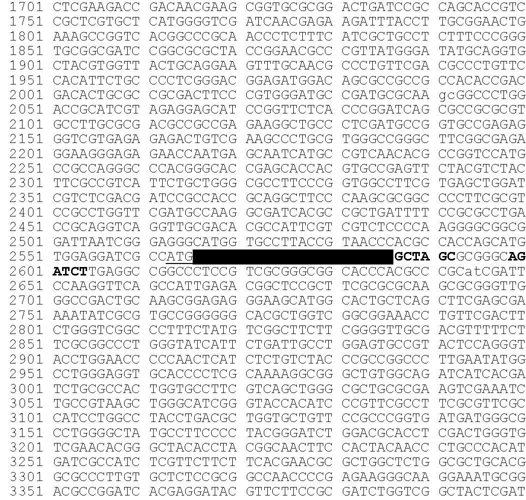

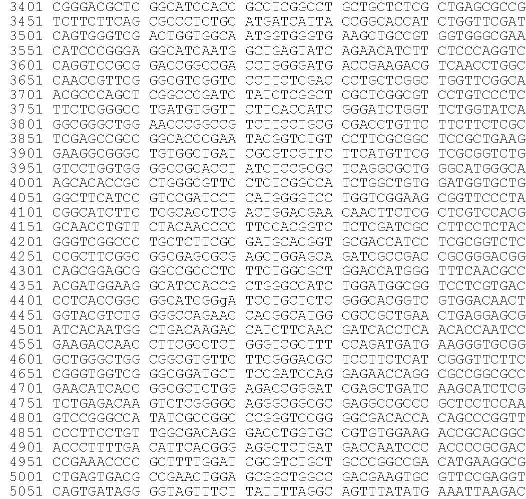

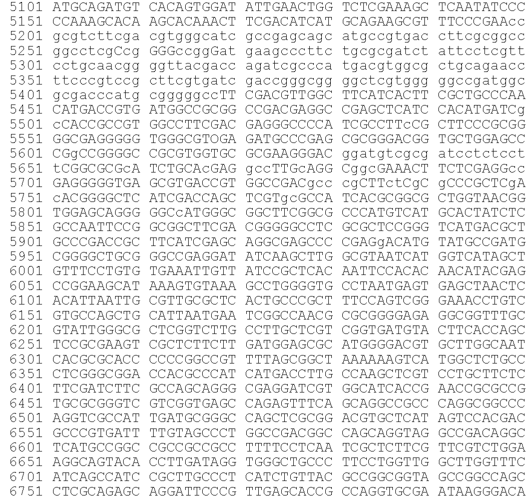

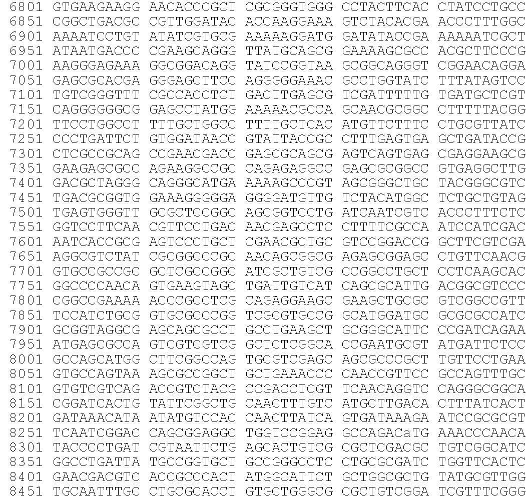

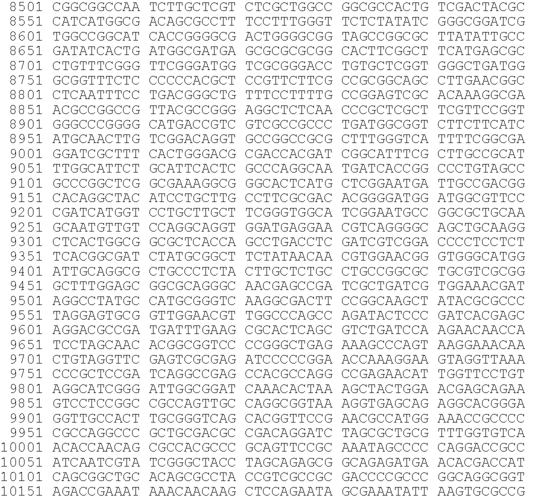

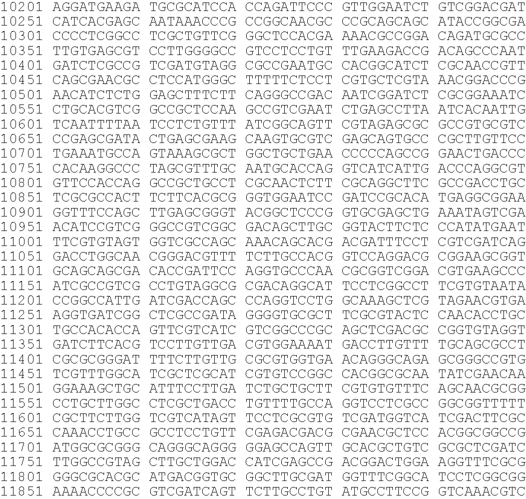

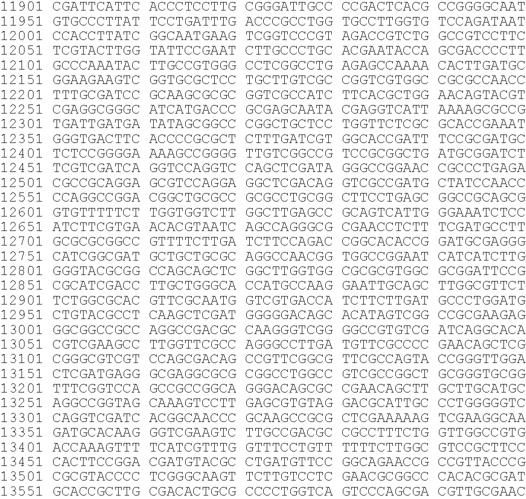

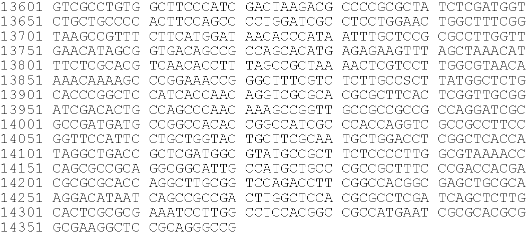

Broad-host-range expression vector for ligation-independent cloning
featuring N-terminal 7 ± His tag followed by cleavage site for Tobacco Etch
Virus protease
pRKHTLICTEV1Dpuf.seq Length: 14398

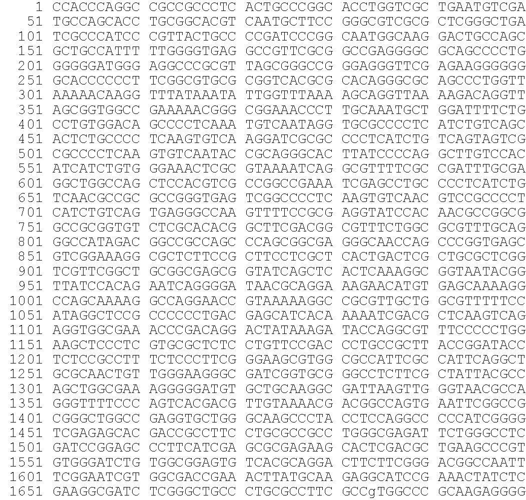

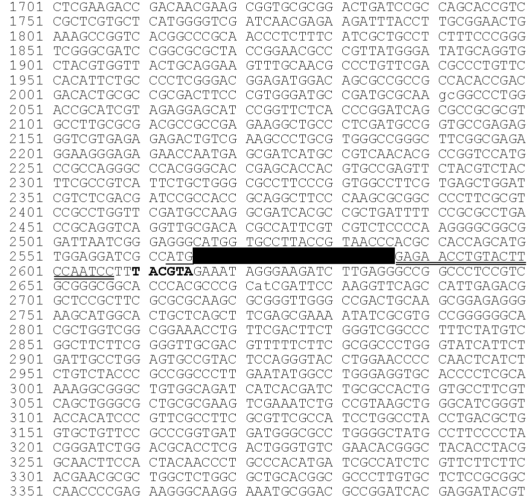

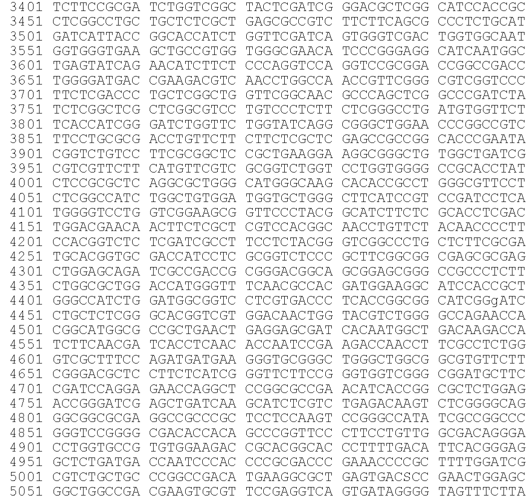

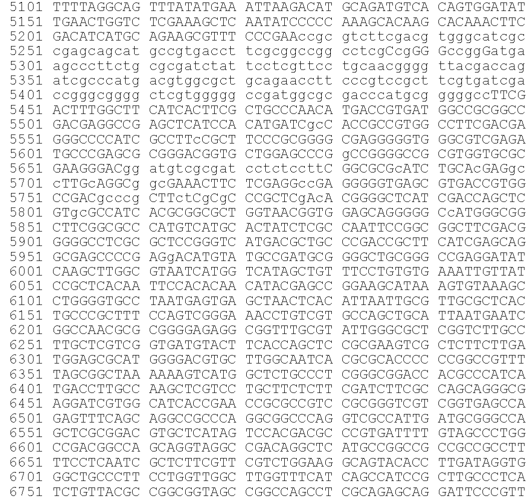

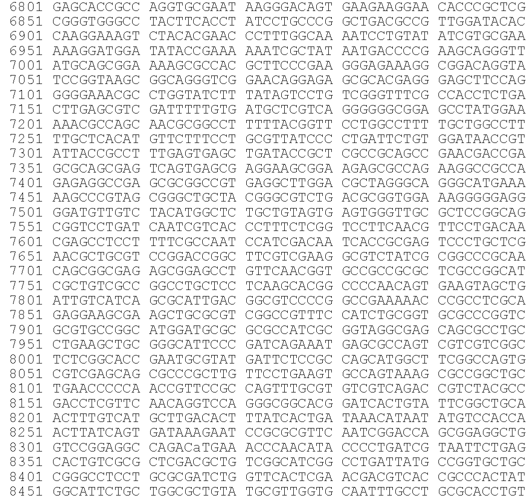

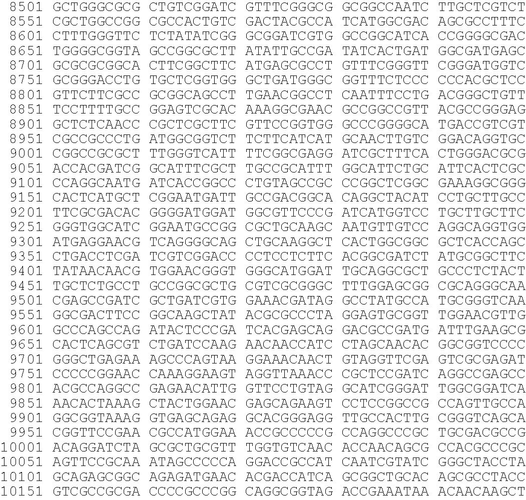

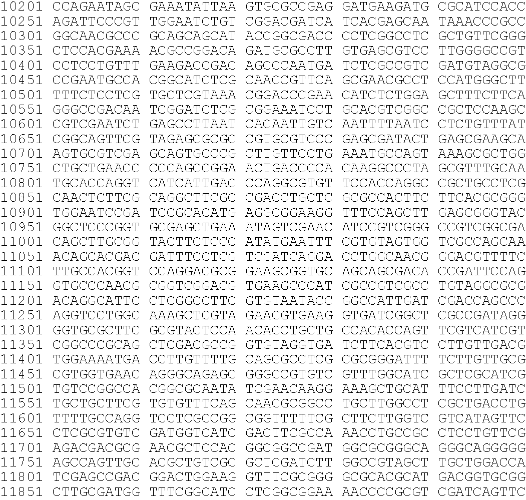

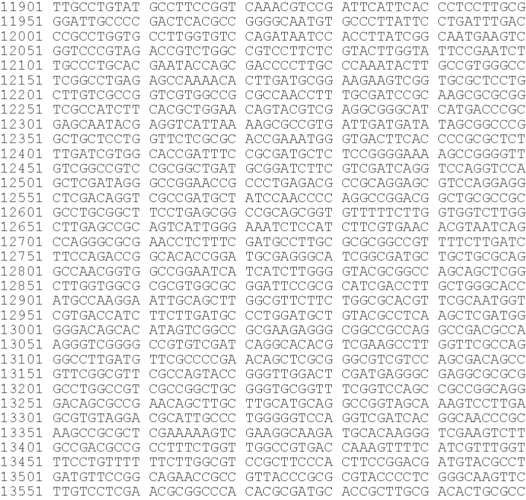

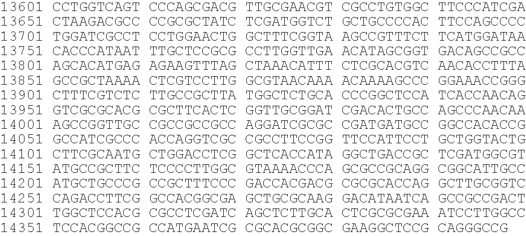

Broad-host-range expression vector with N-terminal 7 ± His tag followed by
cleavage site for Tobacco Etch Virus protease
pRKHTTEVPL1Dpuf.seq Length: 14391

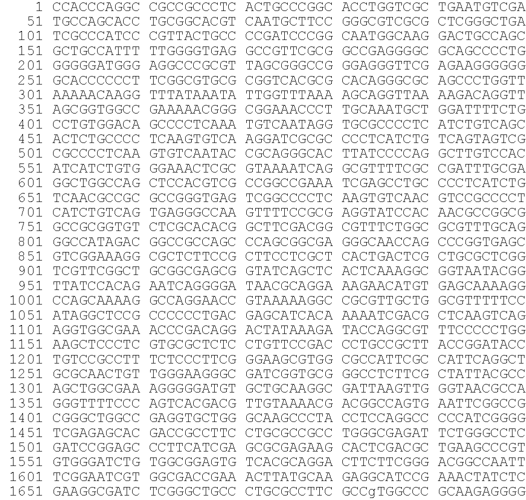

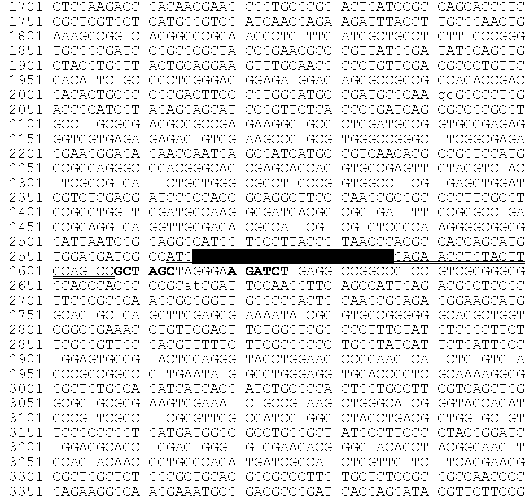

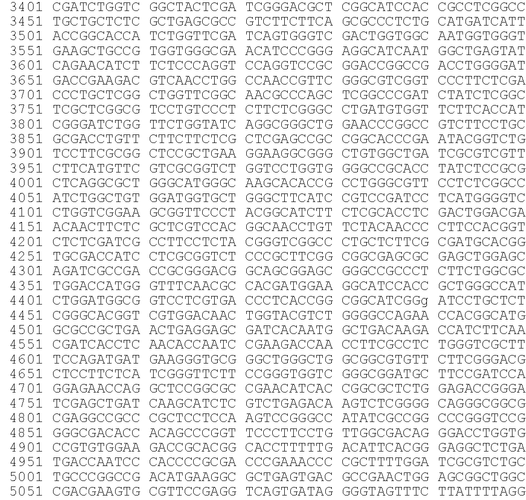

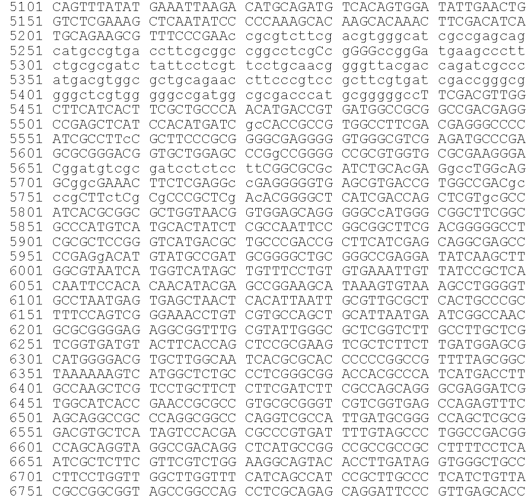

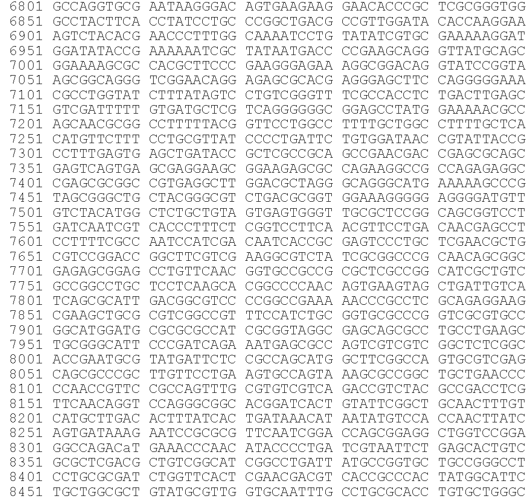

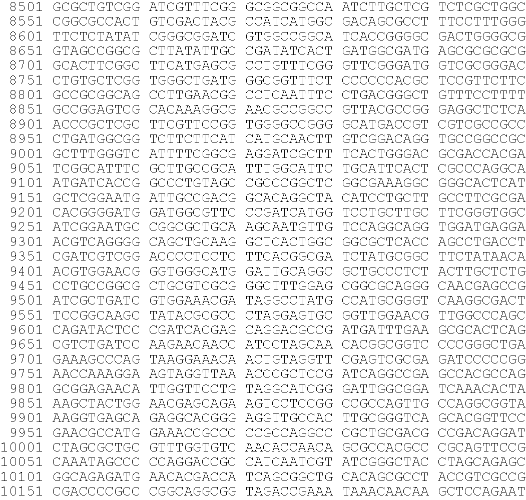

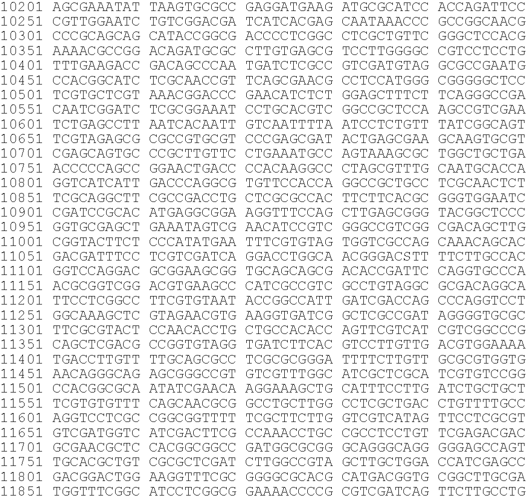

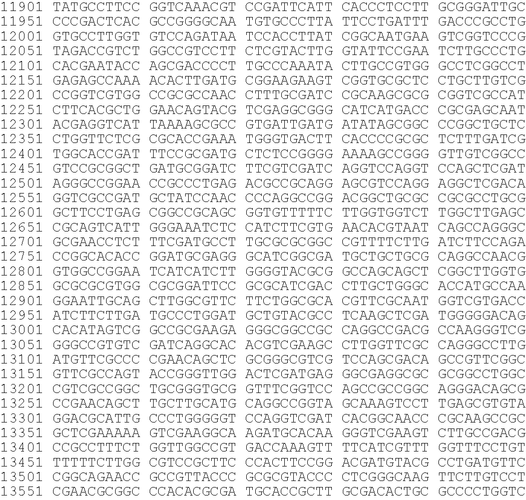

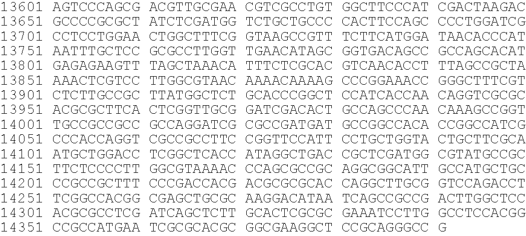

Broad-host-range expression vector for ligation-independent cloning
featuring C-terminal 10 ± His tag
pRKLIC2HT10Dpuf.seq Length: 14377

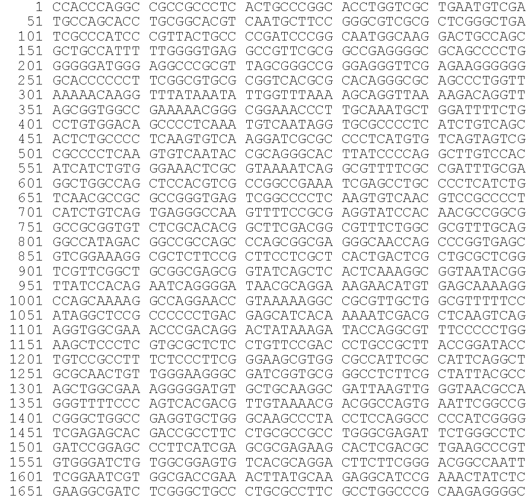

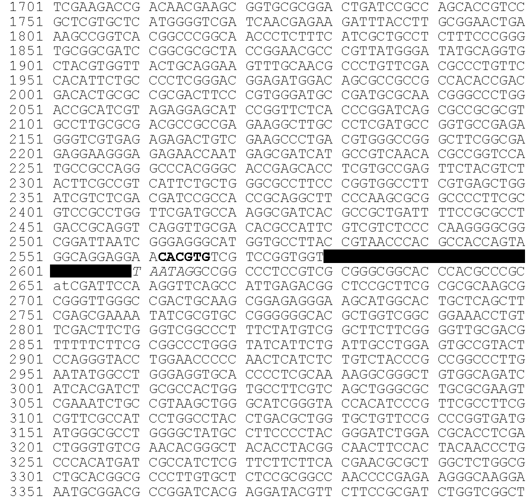

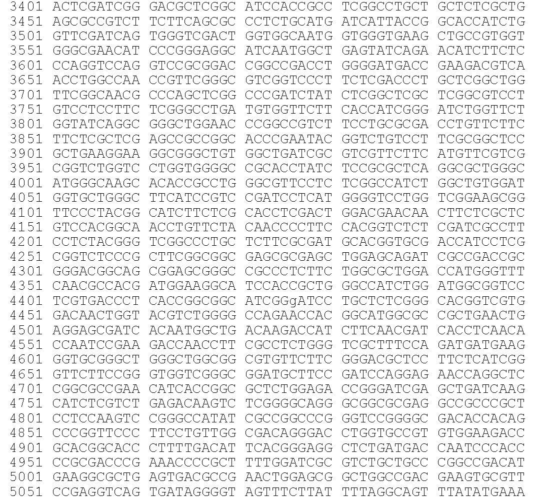

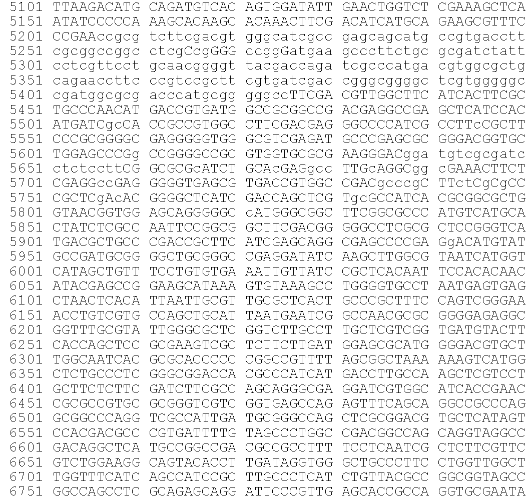

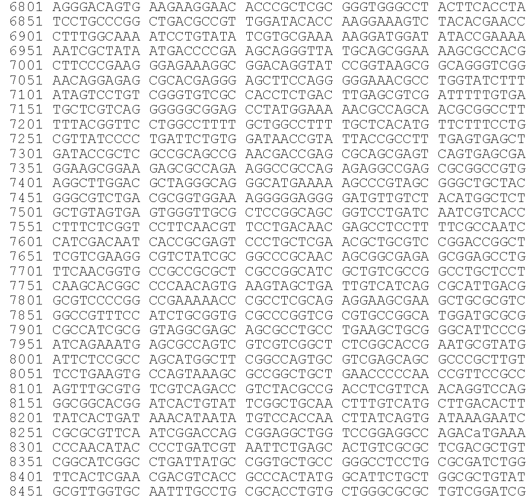

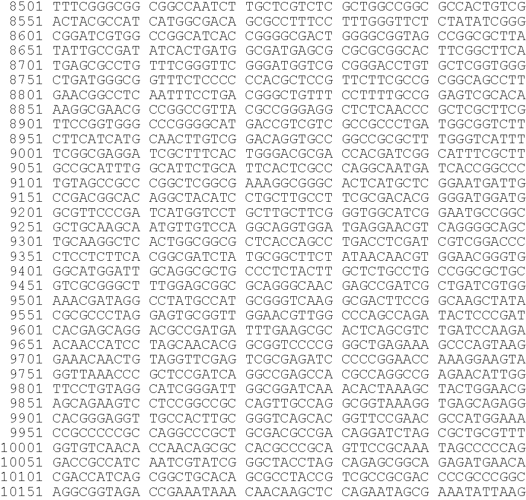

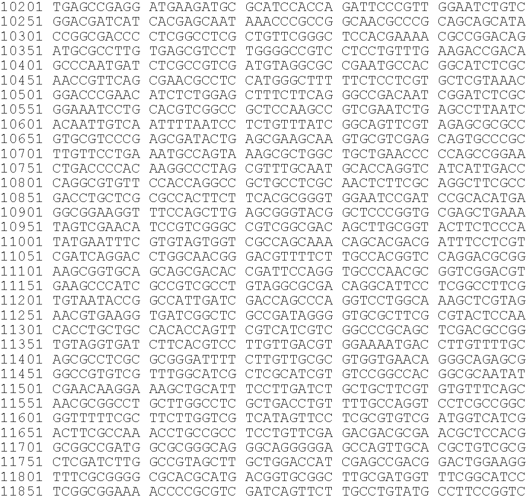

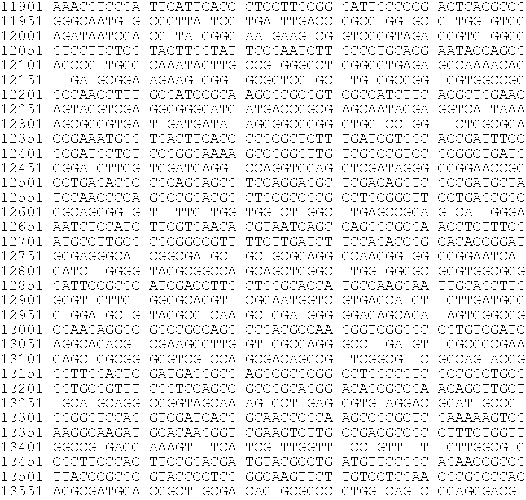

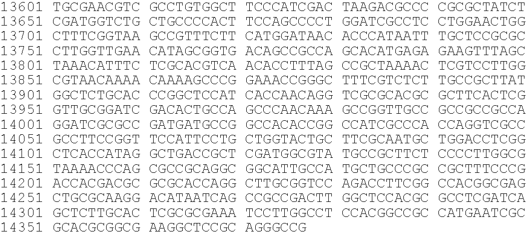

Broad-host-range expression vector for ligation-independent cloning
featuring C-terminal 13 ± His tag
pRKLIC2HT13Dpuf.seq Length: 14386

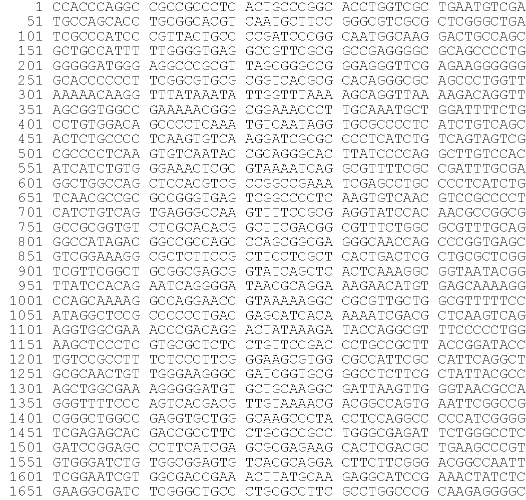

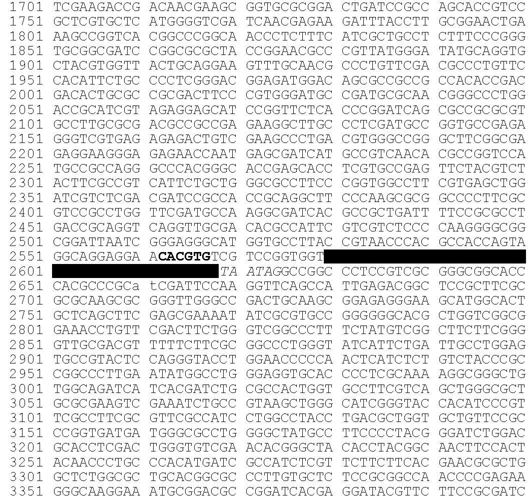

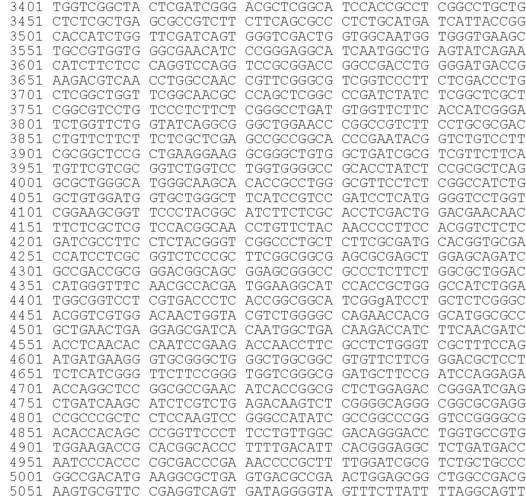

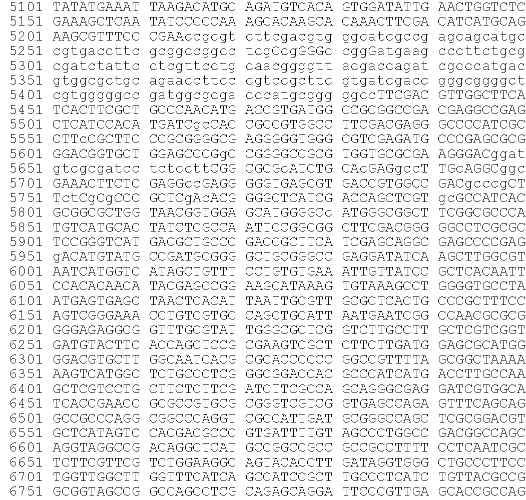

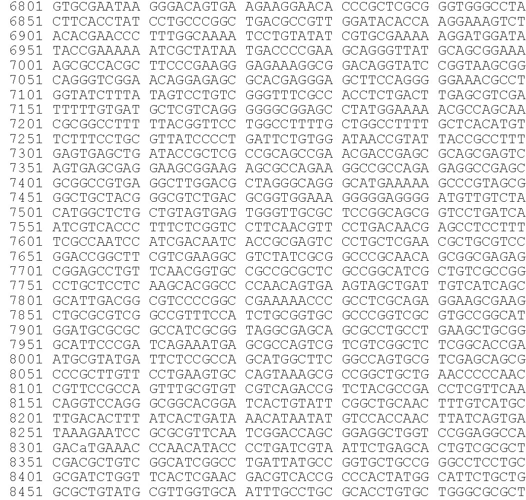

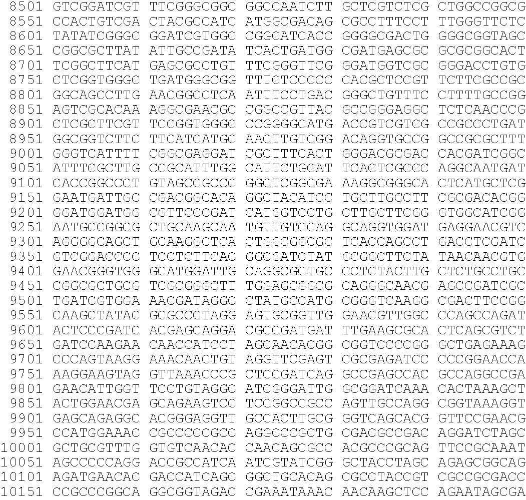

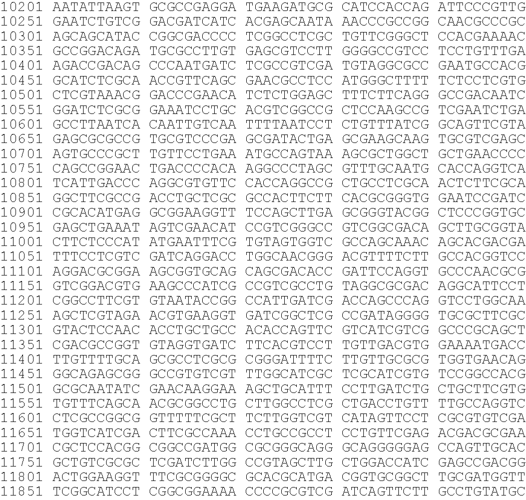

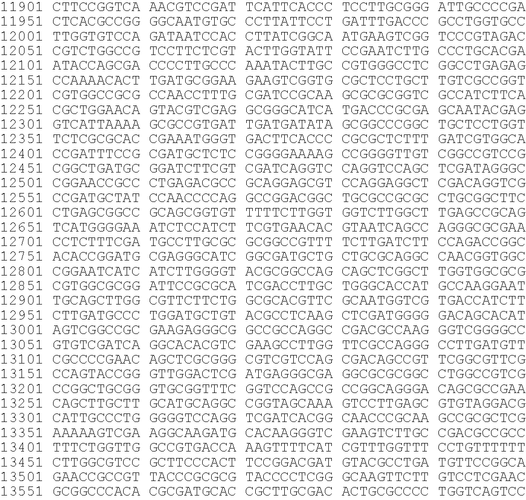

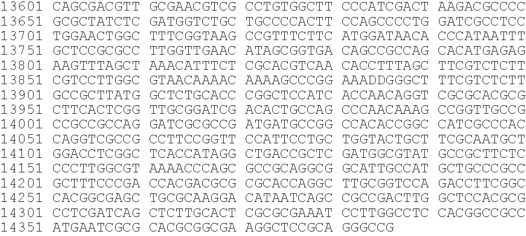

Broad-host-range expression vector for ligation-independent cloning
featuring C-terminal 7 ± His tag
pRKLIC2HT1Dpuf.seq Length: 14368

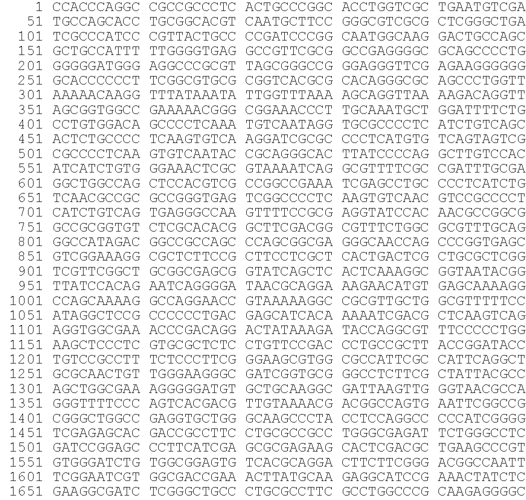

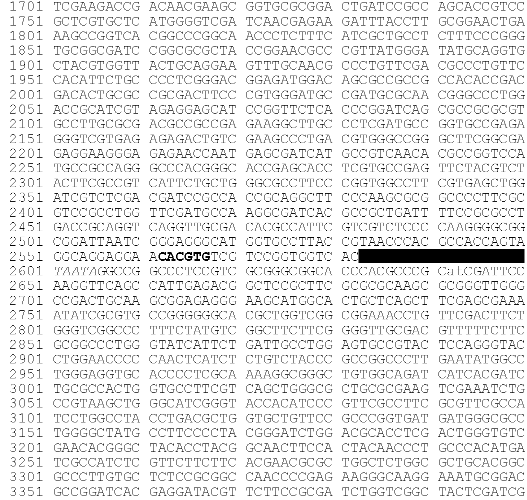

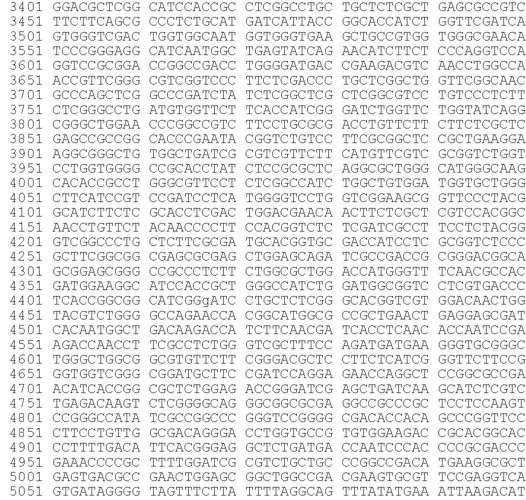

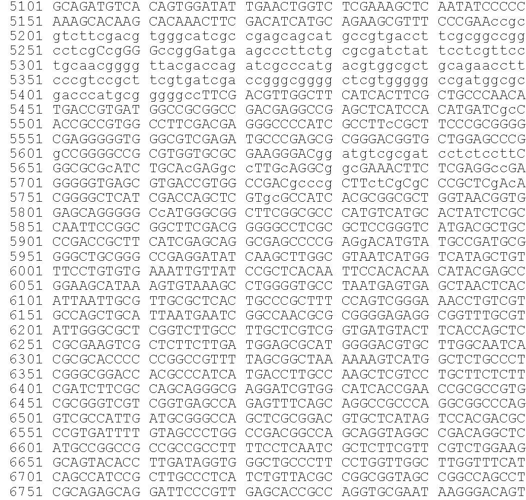

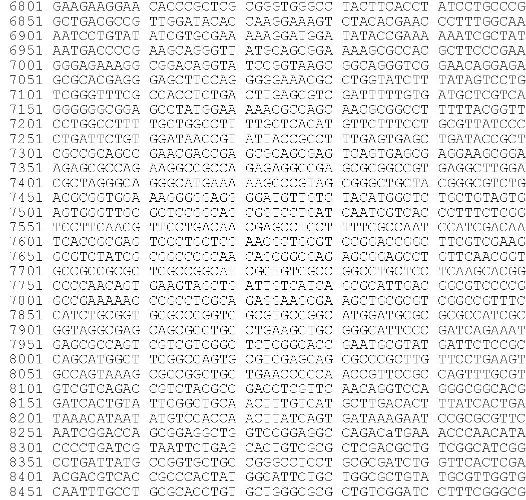

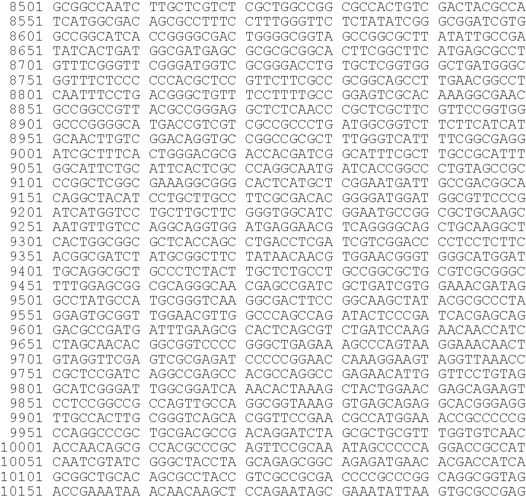

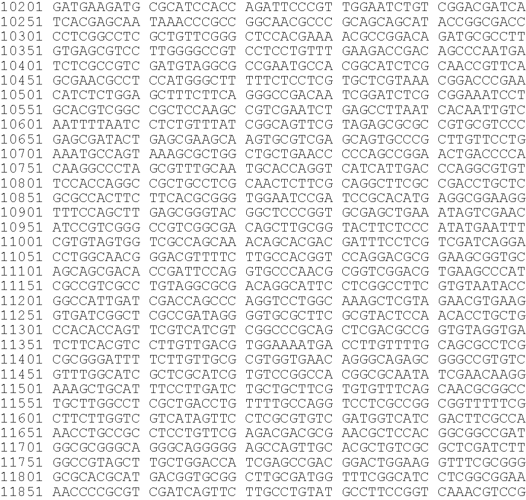

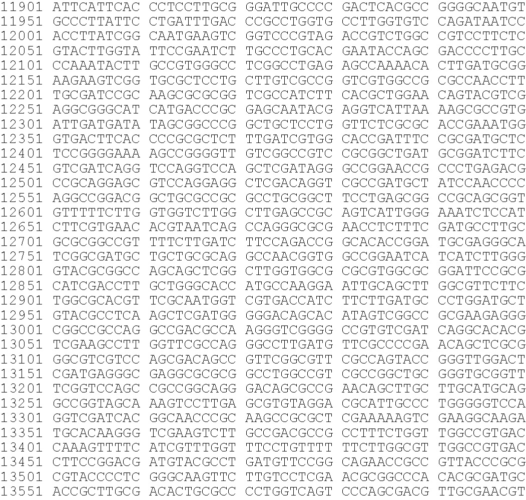

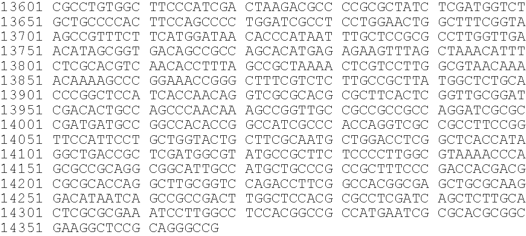

Broad-host-range expression vector featuring C-terminal 10 ± His tag
pRKPLHT10Dpuf Length: 14375

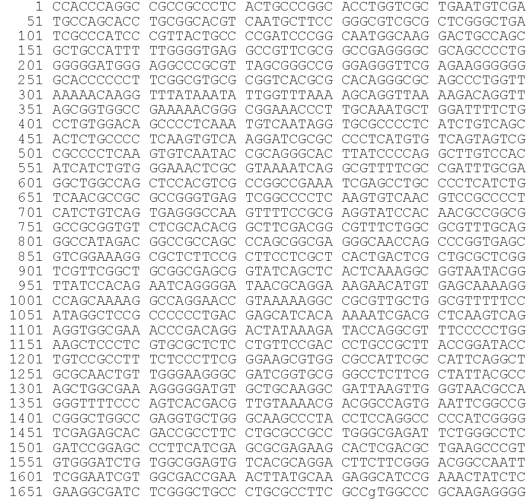

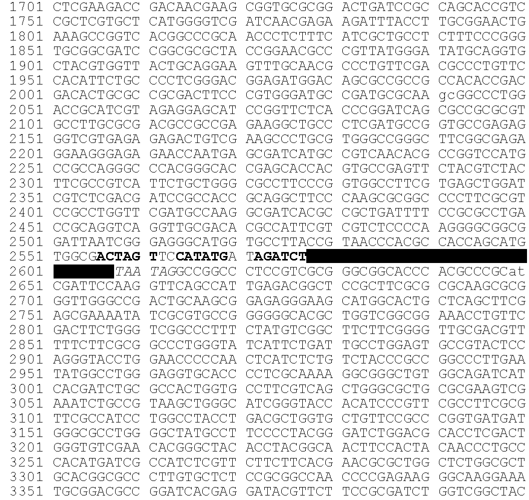

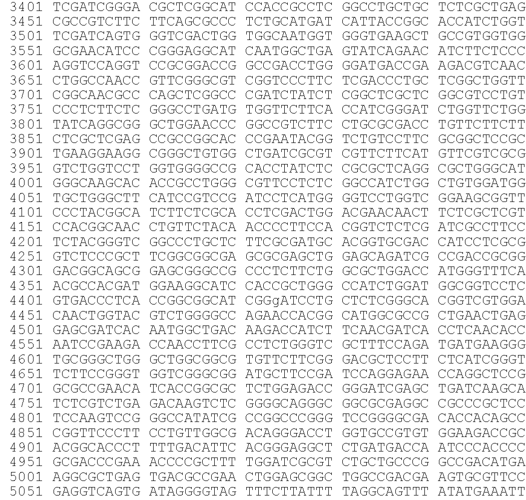

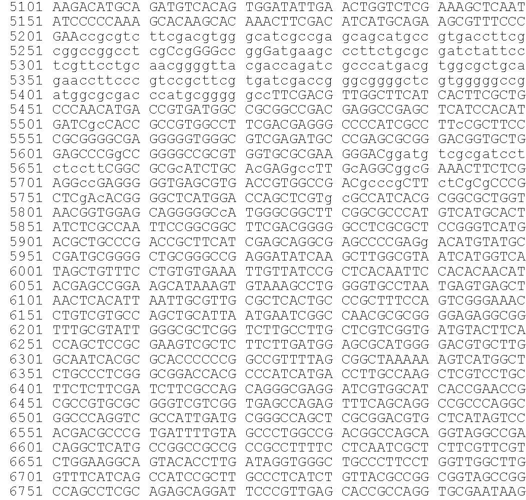

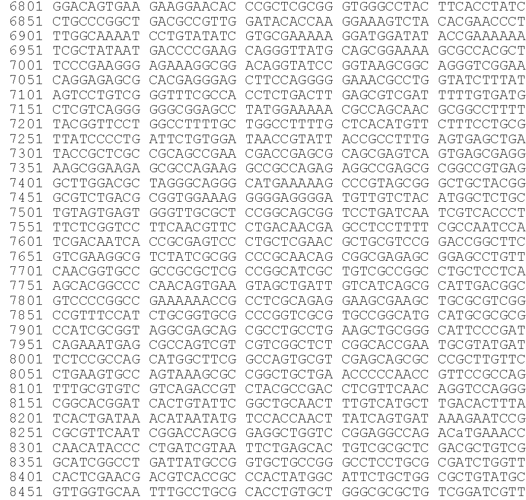

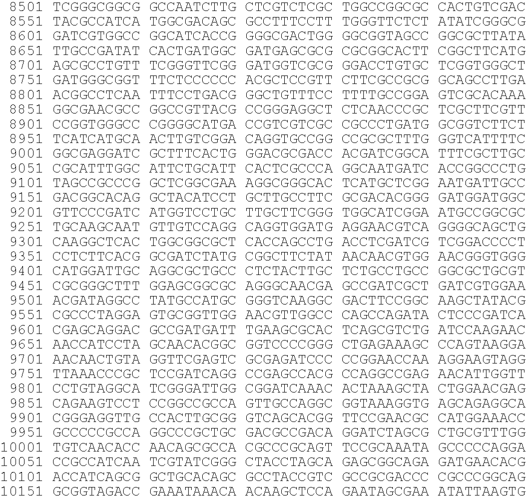

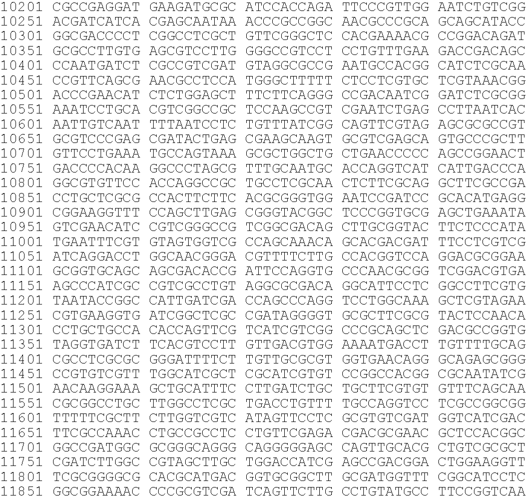

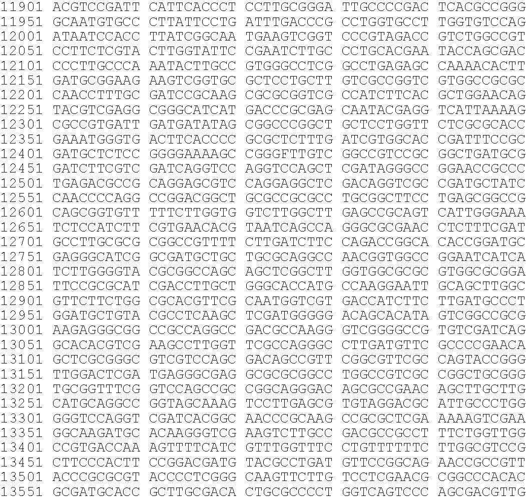

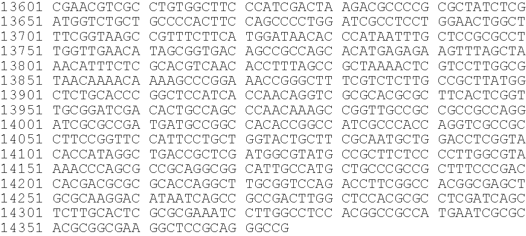

Broad-host-range expression vector featuring C-terminal 13 ± His tag
pRKPLHT13Dpuf.seq Length: 14384

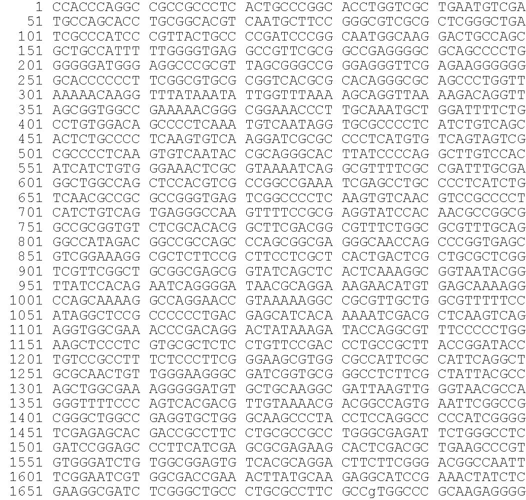

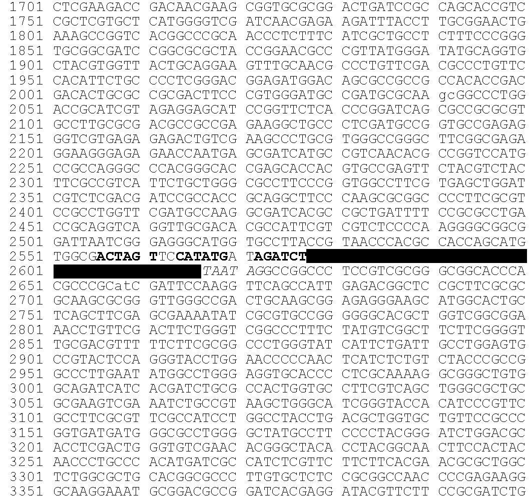

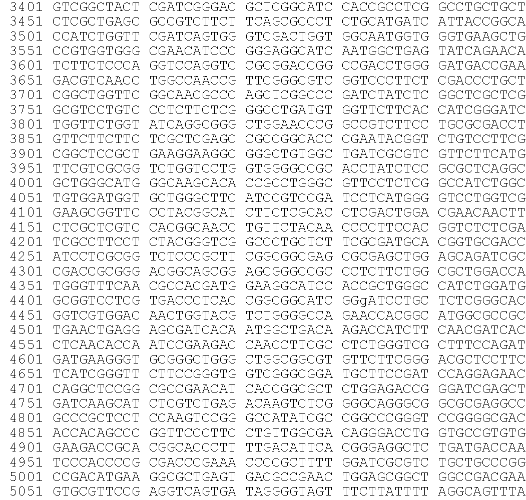

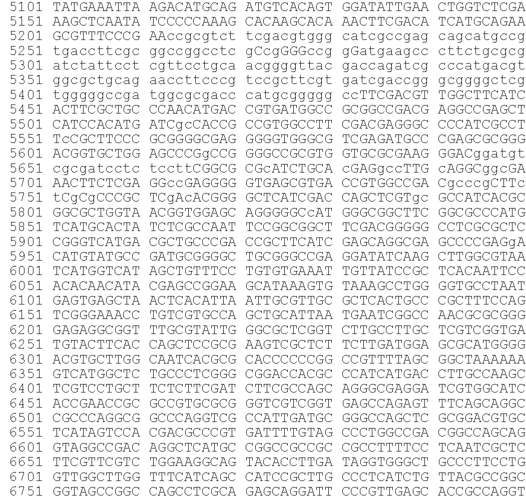

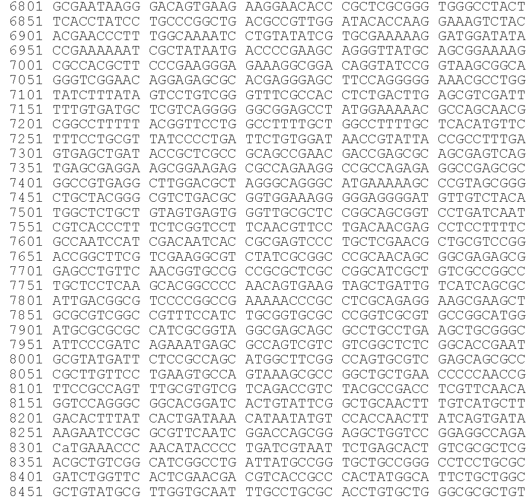

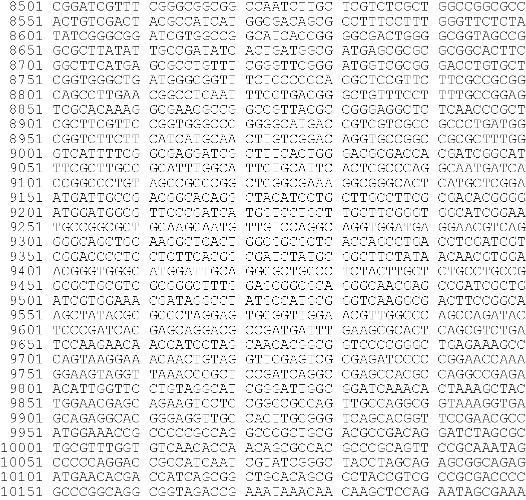

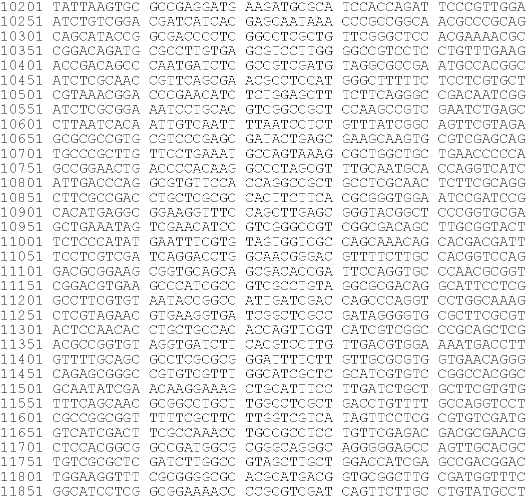

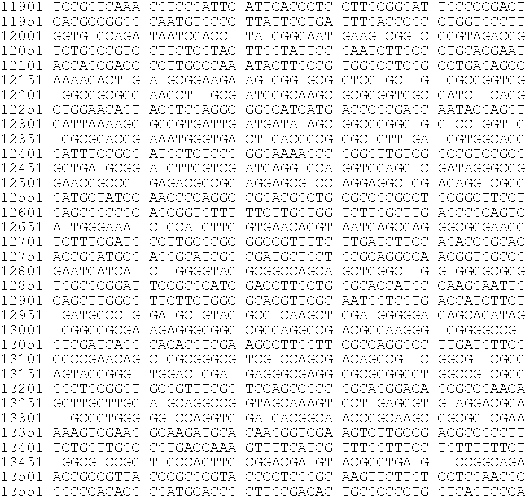

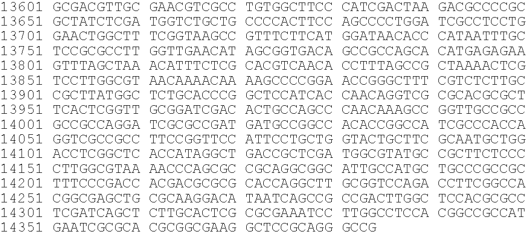

pRKMAHT1Dpuf.seq

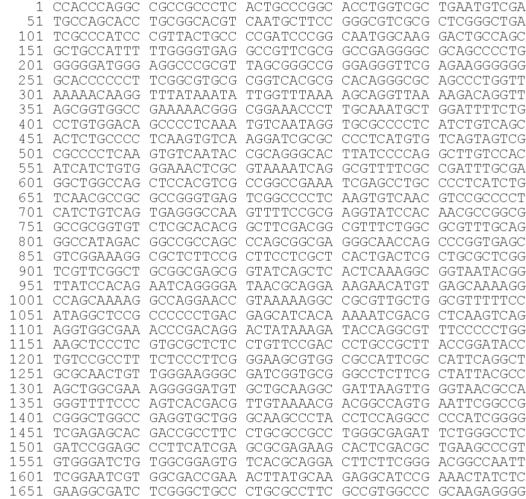

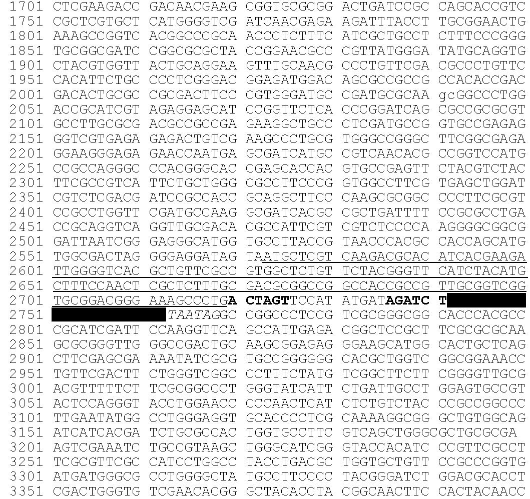

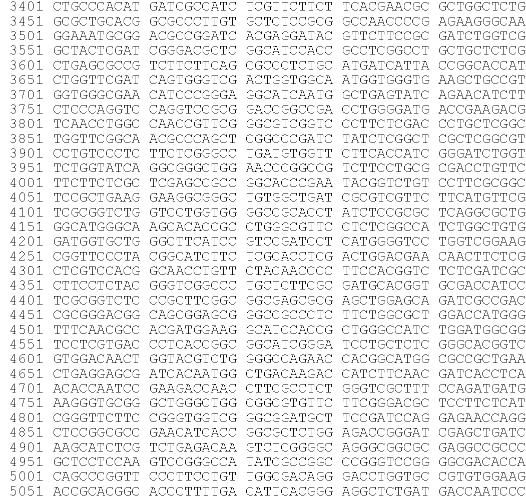

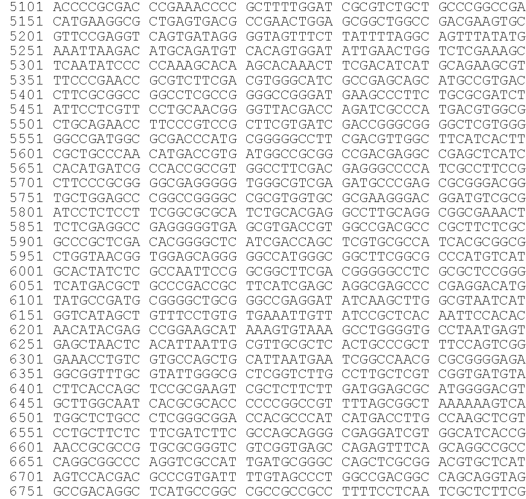

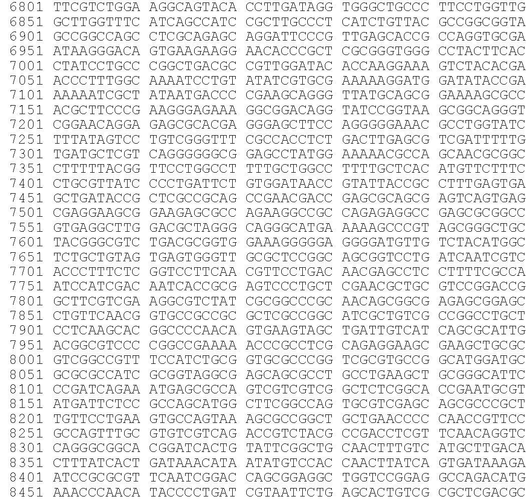

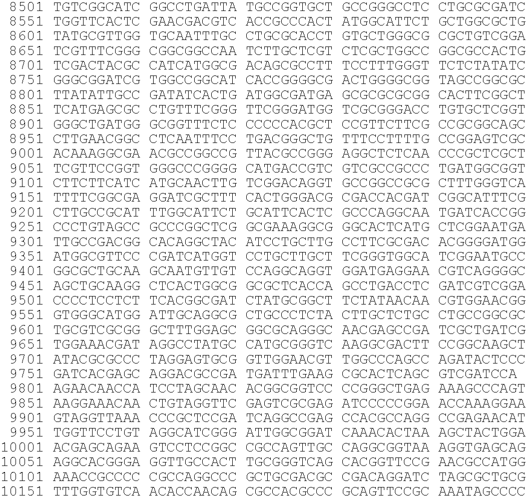

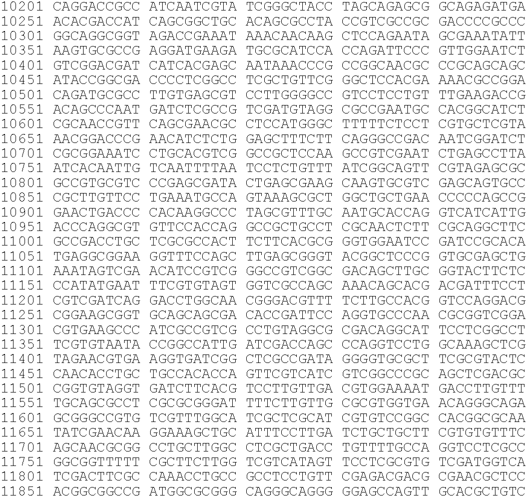

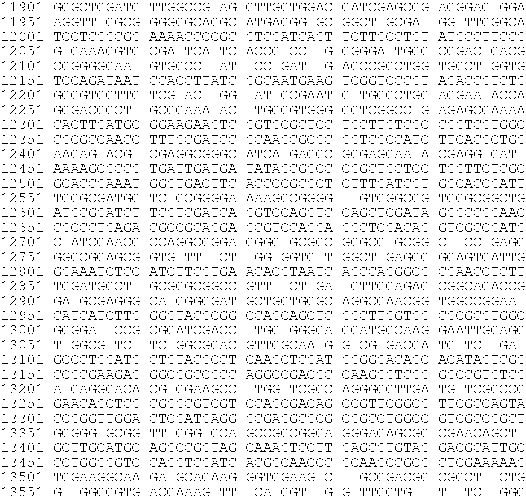

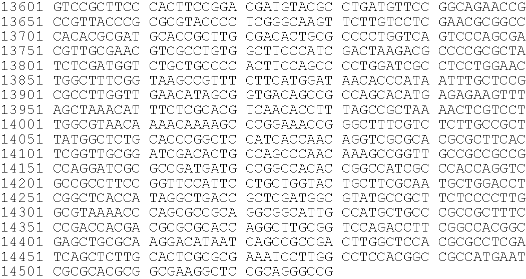

PRKSSHT1DPUF.SEQ

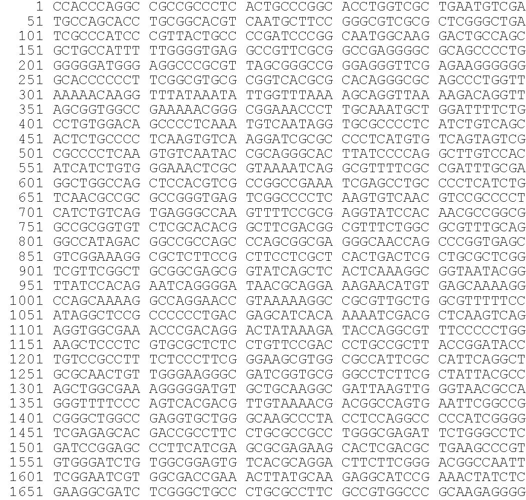

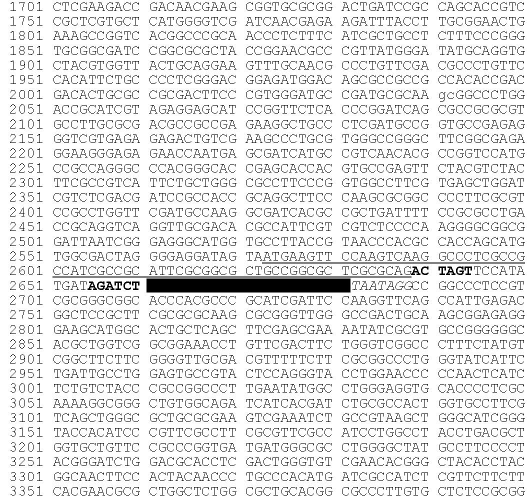

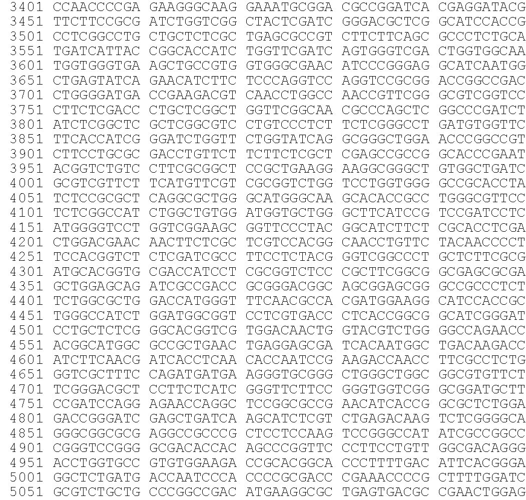

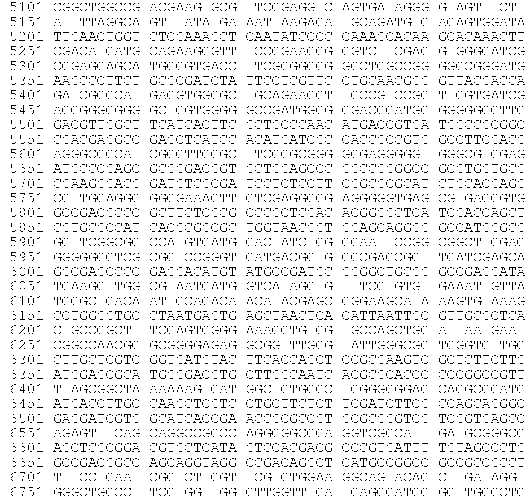

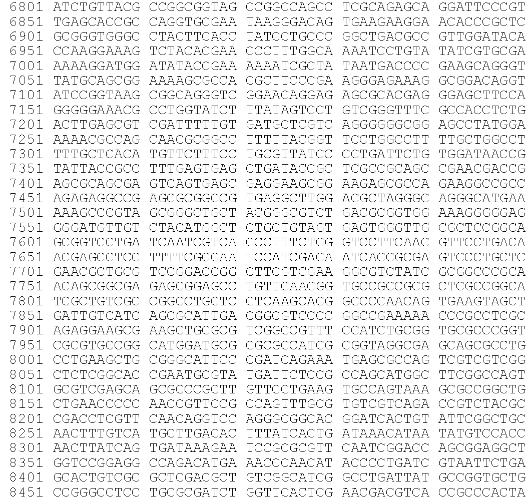

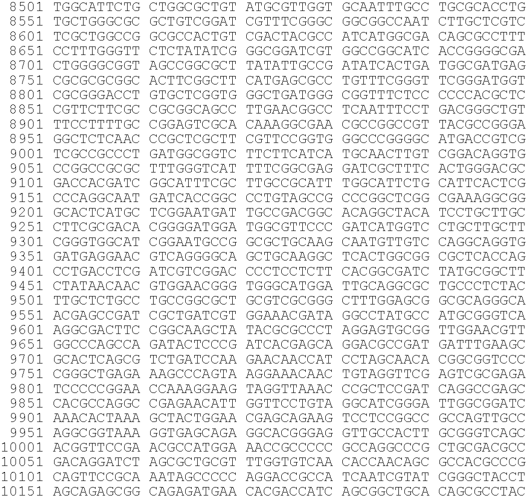

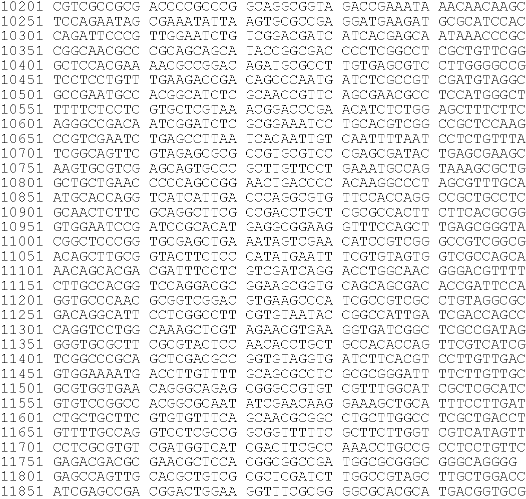

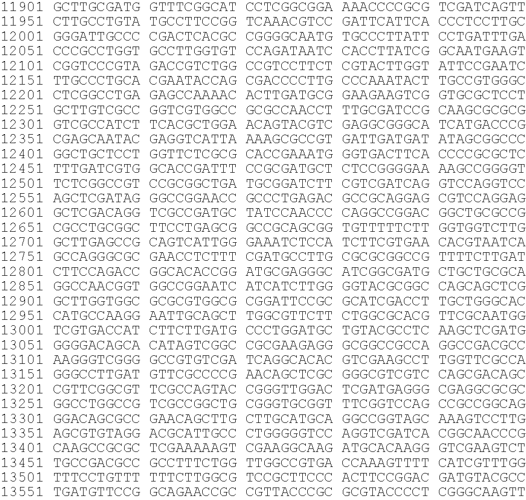

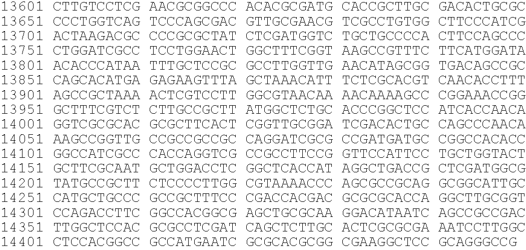

### DOCUMENTS CITED

1. Ditta, G., Schmidhauser, T., Yakobsen, E., Lu, P., Liang, X.-W., Finlay, D. R., GUiney, D., and Helinski, D. R. (1985). Plasmids rtelated to the broad host range vector, pRK290, useful for gene cloning and for monitoring gene expression. Plasmid 13, 149-153.

2. Scott, H. N., Laible, P. D., and Hanson, D. K. (2003). Sequences of versatile broad-host-range vectors of the RK2 family. Plasmid 50, 74-79.

3. Antoine, R., and Locht, C. (1992). Isolation and molecular characterization of a novel broad-host-range plasmid from Bortadella bronchiseptica with sequence similarities to plasmids from Gram-positive organisms. Molecular Microbiology 6, 1785-1799.

4. Kovach, M. E., Phillips, R. W., Elzer, P. H., Roop, R. M., and Peterson, K. M. (1994). pBBR1MCS: Broad host range cloning vector. Biotechniques 16, 800-802.

5. Kovach, M. E., Elzer, P. H., Hill, D. S., Robertson, G. T., Farris, M. A., Roop II, R. M., and Peterson, K. M. (1995). Four new derivatives of the broad-host-range cloning vector pBBR1MCS, carrying different antibiotic-resistance cassettes. Gene 166, 175-176.

6. DeShazer, D., Woods, D. E. (1996). Broad-host-range cloning and cassette vectors based on the R388 trimethoprim resistance gene. Biotechniques 20, 762-764.

7. J. K. Lee et al. (1989)Post-transcriptional control of puc operon expression of B800-850 light-harvesting complex formation in Rhodobacter sphaeroides J. Bacteriol. 171, 3391-3405.

8. J. O. Goldsmith, and S. G. Boxer, (1996) Biochim. Biophys. Acta, 1276, 171-175.

9. C. Aslanidis et al., (1990) Nucleic Acids Res. Ligation-independent cloning of PCR products (LIC-PCR) 18, 6069-6074

10. Yoon, J. R., et al. (2002) Express primer tool for high-throughput gene cloning and expression. BioTechniques 33: 1-5.

11. Pokkuluri et al. (2002) The structure of a mutant photosynthetic reaction center shows unexpected changes in main chain orientations and quinone position. Biochemistry 41: 5998-6007.

12. H. Myllykallio, F. E. Jenney, Jr., C. R. Moomaw, C. A. Slaughter, and F. Daldal. Cytochrone c_{y} of Rhodobacter capsulatus is attached to the cytoplasmic membrane by an uncleaved signal sequence-like anchor. J. Bacteriol. 179:2623-2631 (1997).

13. B. J. MacGregor and T. J. Donohue. Evidence for two promoters for the cytochrone C₂ gene (cycA) of Rhodobacter sphaeroides. J. Bacteriol. 173:3949-3957 (1991).

14. H. Myllykallio, F. E. Jenney, Jr., C. R. Moomaw, C. A. Slaughter, and F. Daldal. Cytochrome c_{y} of Rhodobacter capsulatus is attached to the cytoplasmic membrane by an uncleaved signal sequence-like anchor. J. Bacteriol. 179: 2623-2631 (1997).

15. B. J. MacGregor and T. J. Donohue. Evidence for two promoters for the cytochrome C₂ gene (cycA) of Rhodobacter sphaeroides. J. Bacteriol. 173: 3949-3957 (1991).

We claim:

1. A versatile broad host-range heterologous protein expression vector comprising:
(a) a promoter nucleic acid sequence operable in a photosynthetic bacteria;
(b) a nucleic acid sequence encoding an extended purification tag;
(c) a cloning cassette comprising a multiple cloning site; and
(d) a selection marker to select in the photosynthetic bacteria.

2. The vector of claim 1, wherein the photosynthetic bacteria is Rhodobacter.

3. The vector of claim 1, wherein the extended purification tag is N-terminal to the heterologous protein.

4. The vector of claim 1, wherein the extended purification tag is C-terminal to the heterologous protein.

5. The vector of claim 1, wherein the extended purification tag is a histidine tag comprising about 7 to about 13 contiguous histidine residues.

6. The vector of claim 1, wherein the purification tag comprises a linker sequence.

7. The vector of claim 6, wherein the linker sequence comprises about 1 to about 20 amino acids.

8. The vector of claim 1 further comprises a cleavable signal sequence.

9. The vector of claim 1 further comprises a membrane anchor domain.

10. The vector of claim 1, wherein the cloning cassette facilitates ligation independent cloning.

11. The vector of claim 1, wherein the heterologous protein is a membrane protein.

12. The vector of claim 1, wherein the heterologous protein is a soluble protein.

13. The vector of claim 1 further comprises a nucleic acid sequence encoding a component of an intracytoplasmic membrane of Rhodobacter.

14. The vector of claim 1, wherein the promoter is inducible.

15. A method of producing a heterologous protein in a photosynthetic organism, the method comprising:
(a) cloning a nucleic acid sequence encoding the heterologous protein into a vector of claim 1;
(b) expressing the heterologous protein in a photosynthetic bacteria;
(c) purifying the heterologous protein using an extended purification tag; and
(d) obtaining heterologous protein from the photosynthetic organism.

16. The method of claim 15, wherein the photosynthetic bacteria is Rhodobacter.

17. The method of claim 15, wherein the extended purification tag is a histidine tag comprising about 7 to about 13 contiguous histidine residues.

18. The method of claim 15, wherein the extended purification tag is a histidine tag comprising about 7 to about 13 contiguous histidine residues and further comprising about 1 to about 20 linker amino acids.

19. The method of claim 15, wherein the heterologous protein is a membrane protein.

20. The method of claim 15, wherein the heterologous protein is a soluble protein.

21. A method of producing a heterologous membrane protein in Rhodobacter, the method comprising:
(a) cloning a nucleic acid sequence encoding the heterologous membrane protein into a vector comprising a promoter sequence operable in Rhodobacter, an N- or C-terminal extended purification tag comprising about 7 to about 30 amino acids in length;
(b) expressing the heterologous membrane protein in the Rhodobacter;
(c) sequestering and compartmentalizing the heterologous membrane protein into an intracytoplasmic membrane (ICM) complex;
(d) purifying the heterologous membrane protein using the extended purification tag; and
(e) obtaining heterologous membrane protein from the Rhodobacter.

22. A method of producing a heterologous soluble protein in Rhodobacter, the method comprising:
(a) cloning a nucleic acid sequence encoding the heterologous soluble protein into a vector comprising a promoter sequence operable in Rhodobacter, an N- or C-terminal extended purification tag comprising about 7 to about 30 amino acids in length, and a membrane anchor or linker sequence;
(b) expressing the heterologous soluble protein in the Rhodobacter;
(c) purifying the heterologous soluble protein using the extended purification tag; and
(d) obtaining heterologous soluble protein from the Rhodobacter.
